(19) United States
(12) Patent Application Publication
(10) Pub. No.: US 2025/0313900 A1
(43) Pub. Date: Oct. 9, 2025

(54) COMPOSITIONS AND THERAPEUTIC AGENTS FOR MODULATING INFLAMMATORY RESPONSES ON BOTH TUMOR AND IMMUNE CELLS

(71) Applicant: THE METHODIST HOSPITAL, Houston, TX (US)

(72) Inventors: Shu-Hsia CHEN, Houston, TX (US); Kyeongah KANG, Houston, TX (US); Ping-Ying PAN, Houston, TX (US)

(21) Appl. No.: 18/873,107

(22) PCT Filed: Jun. 12, 2023
(86) PCT No.: PCT/US2023/068302

§ 371 Date: Dec. 9, 2024

Related U.S. Application Data
(60) Provisional application No. 63/351,189, filed on Jun. 10, 2022.

(51) Int. Cl. C12Q 1/6886; A61K 45/06; A61P 35/02; C12N 15/113

(52) U.S. Cl. CPC C12Q 1/6886; A61K 45/06; A61P 35/02; C12N 15/1136; C12N 2310/14; C12N 2320/30; C12Q 2600/112; C12Q 2600/158

(57) ABSTRACT

The relationship between macrophage polarization and cancer progression depending on tumor location and microenvironment has been elusive. New agents and methods for directing macrophage polarization and modulating the host immune response are needed. Disclose are compositions and methods related to the detection, prognosis and treatment of a cancer related to the expression level of CMTM4. In one aspect, the disclosure provides for siRNAs and small molecules that can inhibit CMTM4 for the treatment of cancer.

### II. CROSS REFERENCE TO RELATED APPLICATIONS

This application claims the benefit of U.S. Provisional Application No. 63/351,189, filed on Jun. 10, 2022, which is incorporated herein by reference in its entirety.

### I. GOVERNMENT SUPPORT

This invention was made with government support under Grant Nos. CA204191 and CA208703 awarded by the National Institutes of Health. The government has certain rights in the invention

### III. REFERENCE TO SEQUENCE LISTING

The sequence listing submitted on Jun. 12, 2023, conforming to the rules of WIPO Standard ST.26 as an .XML file entitled “10063-072WO1.XML” created on Jun. 12, 2023, and having a file size of 13474 bytes is hereby incorporated by reference pursuant to 37 C.F.R. § 1.52(e)(5).

### IV. BACKGROUND

Cancer is a multifaceted disease influenced by both environmental and genetic factors. The immune system plays an important role in not only the promotion but also inhibition of cancer development. Macrophages are a major component of the leukocyte infiltrate of tumors. The infiltration of macrophages into the tumor can exacerbate cancer symptoms as well as reduce tumorigenesis, indicating critical roles of macrophages in the tumor microenvironment. Therefore, the function of macrophages can dictate the outcome of cancer development.

Immature macrophages or myeloid-derived suppressor cells (MDSC) exhibit functional plasticity and have various functions in the immune system. These myeloid cells play an important role in the initiation and resolution of inflammatory responses and can be differentiated in response to the microenvironmental stimuli and acquire pro-inflammatory or anti-inflammatory phenotypes. Macrophages can be categorized functionally into two major distinct phenotypes, classically (M1) or alternatively (M2) activated macrophages. Although the separation of macrophages into M1 and M2 subtypes are likely to represent a somewhat inexact and artificial classification, our laboratory and others have demonstrated that M1 macrophages exert antitumor immunity whereas M2 macrophages play various roles in tumor progression. M1 macrophages efficiently kill cancer cells through phagocytosis and cytotoxicity while M2 macrophages promote tissue repair, angiogenesis, and tumor growth. Paradoxically, M1 macrophages may also play a tumor-promoting role, depending on at which stage of tumorigenesis whereas M2 macrophages denote lower tumor malignancy and increased survival. Especially, the pro- and anti-tumor roles of macrophages may differ in tumor stages, e.g., initiation versus growth of tumors. The molecular mechanisms of macrophage polarization have not been fully delineated. Furthermore, the relationship between macrophage polarization and cancer progression depending on tumor location and microenvironment has been elusive. What are needed are new agents and methods for directing macrophage polarization and modulating the host immune response.

### V. SUMMARY

Disclosed are methods and compositions related to the detection, prognosis and treatment of a cancer related to the expression level of CMTM4.

In one aspect, disclosed herein are methods of detecting a cancer (such as, for example, cholangiocarcinoma (CHOL), colon adenocarcinoma (COAD), esophageal carcinoma (ESCA), kidney chromophobe (KICH), pheochromocytoma and paraganglioma (PCPG), prostate adenocarcinoma (PRAD), rectum adenocarcinoma (READ), and thymoma (THYM), breast cancer, colon cancer, melanoma, prostate cancer, a glioma, kidney cancer, or lung adenocarcinoma) in a subject comprising obtaining a tissue sample from the subject and measuring the expression level of chemokine-like factor (CKLF)-like MARVEL transmembrane domain-containing member 4 (CMTM4) relative to a control, wherein an increase in the expression of CMTM4 relative to the control indicates the presence of a cancer.

Also disclosed herein are methods of assessing the aggressiveness/severity of a cancer (such as, for example, cholangiocarcinoma (CHOL), colon adenocarcinoma (COAD), esophageal carcinoma (ESCA), kidney chromophobe (KICH), pheochromocytoma and paraganglioma (PCPG), prostate adenocarcinoma (PRAD), rectum adenocarcinoma (READ), and thymoma (THYM), breast cancer, colon cancer, melanoma, prostate cancer, a glioma, kidney cancer, or lung adenocarcinoma) and/or whether a cancer in a subject is metastatic comprising obtaining a cancerous tissue sample from a tumor microenvironment in the subject and measuring the expression level of Chemokine-like factor (CKLF)-like MARVEL transmembrane domain-containing member 4 (CMTM4) in the tissue sample relative to a control, wherein an increase in the expression level of CMTM4 relative to the control indicates the cancer is metastatic.

In one aspect, disclosed herein are methods of detecting a cancer of any preceding aspect, methods of assessing the aggressiveness/severity of a cancer of any preceding aspect, or whether a cancer in a subject is metastatic of any preceding aspect; wherein a cancer is detected, or a cancer is found to be metastatic, the method further comprises administering to the subject an agent that inhibits CMTM4. For example, the method can comprise administering a miRNA, shRNA, siRNA (such as, for example, UUAGAUUCCAGUUGAUCUGGG (SEQ ID NO: 1), UGUUAGAUUCCAGUUGAUCUG (SEQ ID NO: 2), ACCAAAUCUGUUAGAUUCCAG (SEQ ID NO: 3), AGAAAACUUGAUUAGAAGGAC (SEQ ID NO: 4), AAGAAAACUUGAUUAGAAGGA (SEQ ID NO: 5), AGAAAGAAAACUUGAUUAGAA (SEQ ID NO: 6), UGAAUUUUUACCAAACAGGAC (SEQ ID NO: 7), AAGUGAAUUUUUACCAAACAG (SEQ ID NO: 8), UUUAUUAAGGUUUUGACUCAU (SEQ ID NO: 9), UAUACUUCCCUUCUCAAUGCC (SEQ ID NO: 10), CTTGATTAGAAGGACGGTT (SEQ ID NO: 11), AGAUCAACUGGAACCUGACAGAUUU (SEQ ID NO: 12), GCCGUGAUAUUUGGCUUCUUGGCAA (SEQ ID NO: 13), or GGCCCUGAUUGCGUUCAUCAUCUGCAUA (SEQ ID NO: 14), peptide, small molecule (including, but not limited to C29H38O4 (Celastrol), Ellipticine, C54H78N2O17 (Lobophorin F), C19H14O7 (5-Methoxysterigmatocysin), C16H12O7 (Isorhamnetin), C21H26N2O3 (Tabernaemontanin), C27H34O10 (Verrucarin A 9,10-epoxide), C10H12N4O5 (Inosine), C16H12O7, C18H23NO6, C25H33N3O·ClH, C16H12O7 (Rhamnetin), C12H13N5O4 (Toyocamycin), C17H19N3O4 (Anthramycin methyl ether), C21H22N2O3 (Paquinimod), C20H18N2O5·Na (Camptothecin sodium), C18H16O8 (Rosmarinic Acid), C10H14O3, C9H16N2O6 (Tetrahydrouridine), C10H14O3 (Mephenesin), C10H14O3 (4-Ethoxy-3-methoxybenzyl alcohol), C10H14O3 (5-tert-butyl-2-methyl-3-furoic acid), C21H20F3NO6, C23H32N2O6, C15H10N20, C36H48N2O12 (Rhoodomycin A), C16H24O5 (Ocalicine subst.), C14H25N3O9 (Kasugamycin), C4H6O2 (Crotonic Acid), C21H26N2O3 (Vincamine), Fumitremorgin C, C10H12N4O5 (Formycin B), C18H23NO6 (Swazine), C25H33N3O·ClH (Butylcycloheptylprodiginine Hydrochloride), C18H22N2O4·C6H8O7 (Quinocarcin monocitrate), C23H28N2O5, C11H13NO2·BrH, C21H24N2O3 (Strychninic acid), C21H22N2O3 (Pseudostrychnine), C20H18N2O5·Na (Camptothecin sodium), C18H16O8 (Centaureidin), C10H14O3 (Ramulosin), C19H26N2·C4H6O6, C15H10N2O6 (Lomondomycin), C21H26N2O3 (Pseudoyohimbine), or C26H28O5 (Glycyrrhizol A)), or antibody that binds to CMTM4, inhibits the activity of CMTM4, or inhibits the expression of CMTM4.

In one aspect, disclosed herein are methods of treating, inhibiting, decreasing, reducing, ameliorating and/or preventing a cancer and/or metastasis (such as, for example, cholangiocarcinoma (CHOL), colon adenocarcinoma (COAD), esophageal carcinoma (ESCA), kidney chromophobe (KICH), pheochromocytoma and paraganglioma (PCPG), prostate adenocarcinoma (PRAD), rectum adenocarcinoma (READ), and thymoma (THYM), breast cancer, colon cancer, melanoma, prostate cancer, a glioma, kidney cancer, or lung adenocarcinoma) in a subject comprising administering to the subject an agent that inhibits CMTM4. For example, the method can comprise administering a miRNA, shRNA, siRNA (such as, for example, UUAGAUUCCAGUUGAUCUGGG (SEQ ID NO: 1), UGUUAGAUUCCAGUUGAUCUG (SEQ ID NO: 2), ACCAAAUCUGUUAGAUUCCAG (SEQ ID NO: 3), AGAAAACUUGAUUAGAAGGAC (SEQ ID NO: 4), AAGAAAACUUGAUUAGAAGGA (SEQ ID NO: 5), AGAAAGAAAACUUGAUUAGAA (SEQ ID NO: 6), UGAAUUUUUACCAAACAGGAC (SEQ ID NO: 7), AAGUGAAUUUUUACCAAACAG (SEQ ID NO: 8), UUUAUUAAGGUUUUGACUCAU (SEQ ID NO: 9), UAUACUUCCCUUCUCAAUGCC (SEQ ID NO: 10), CTTGATTAGAAGGACGGTT (SEQ ID NO: 11), AGAUCAACUGGAACCUGACAGAUUU (SEQ ID NO: 12), GCCGUGAUAUUUGGCUUCUUGGCAA (SEQ ID NO: 13), or GGCCCUGAUUGCGUUCAUCAUCUGCAUA (SEQ ID NO: 14), peptide, small molecule (including, but not limited to C29H38O4 (Celastrol), Ellipticine, C54H78N2O17 (Lobophorin F), C19H14O7 (5-Methoxysterigmatocysin), C16H12O7 (Isorhamnetin), C21H26N2O3 (Tabernaemontanin), C27H34O10 (Verrucarin A 9,10-epoxide), C10H12N4O5 (Inosine), C16H12O7, C18H23NO6, C25H33N3O·ClH, C16H12O7 (Rhamnetin), C12H13N5O4 (Toyocamycin), C17H19N3O4 (Anthramycin methyl ether), C21H22N2O3 (Paquinimod), C20H18N2O5·Na (Camptothecin sodium), C18H16O8 (Rosmarinic Acid), C10H14O3, C9H16N2O6 (Tetrahydrouridine), C10H14O3 (Mephenesin), C10H14O3 (4-Ethoxy-3-methoxybenzyl alcohol), C10H14O3 (5-tert-butyl-2-methyl-3-furoic acid), C21H20F3NO6, C23H32N2O6, C15H10N2O, C36H48N2O12 (Rhoodomycin A), C16H24O5 (Ocalicine subst.), C14H25N3O9 (Kasugamycin), C4H6O2 (Crotonic Acid), C21H26N2O3 (Vincamine), Fumitremorgin C, C10H12N4O5 (Formycin B), C18H23NO6 (Swazine), C25H33N3O·ClH (Butylcycloheptylprodiginine Hydrochloride), C18H22N2O4·C6H8O7 (Quinocarcin monocitrate), C23H28N2O5, C11H13NO2·BrH, C21H24N2O3 (Strychninic acid), C21H22N2O3 (Pseudostrychnine), C20H18N2O5·Na (Camptothecin sodium), C18H16O8 (Centaureidin), C10H14O3 (Ramulosin), C19H26N2·C4H6O6, C15H10N2O6 (Lomondomycin), C21H26N2O3 (Pseudoyohimbine), or C26H28O5 (Glycyrrhizol A)), or antibody that binds to CMTM4, inhibits the activity of CMTM4, or inhibits the expression of CMTM4. In some aspect, the method can further comprise the administration of anti-inflammatory agents (such as, for example, an agent that inhibits LPS, IL-1β, IFNγ, TNF-α, and/or S100A8) and/or antibodies that bind to neutrophils. In one aspect, the method can further comprise the administration of an epidermal growth factor receptor (EGFR) inhibitor (such as, for example, erlotinib, osimertinib, neratinib, gefitinib, cetuximab, pantibumumab, dacomitinib, lapatinib, necitumumab, mobocertinib, and vandetanib) or a platelet-derived growth factor receptor A (PDGFRa) inhibitor (such as, for example, avapritinib, imatinib, and ripretinib).

Also disclosed herein are methods of decreasing immunosuppressive activity or increasing MHCII expression in a tumor microenvironment of a cancer (such as, for example, cholangiocarcinoma (CHOL), colon adenocarcinoma (COAD), esophageal carcinoma (ESCA), kidney chromophobe (KICH), pheochromocytoma and paraganglioma (PCPG), prostate adenocarcinoma (PRAD), rectum adenocarcinoma (READ), and thymoma (THYM), breast cancer, colon cancer, melanoma, prostate cancer, a glioma, kidney cancer, or lung adenocarcinoma) in a subject comprising administering to the microenvironment an agent that inhibits CMTM4. For example, the method can comprise administering a miRNA, shRNA, siRNA (such as, for example, UUAGAUUCCAGUUGAUCUGGG (SEQ ID NO: 1), UGUUAGAUUCCAGUUGAUCUG (SEQ ID NO: 2), ACCAAAUCUGUUAGAUUCCAG (SEQ ID NO: 3), AGAAAACUUGAUUAGAAGGAC (SEQ ID NO: 4), AAGAAAACUUGAUUAGAAGGA (SEQ ID NO: 5), AGAAAGAAAACUUGAUUAGAA (SEQ ID NO: 6), UGAAUUUUUACCAAACAGGAC (SEQ ID NO: 7), AAGUGAAUUUUUACCAAACAG (SEQ ID NO: 8), UUUAUUAAGGUUUUGACUCAU (SEQ ID NO: 9), UAUACUUCCCUUCUCAAUGCC (SEQ ID NO: 10), CTTGATTAGAAGGACGGTT (SEQ ID NO: 11), AGAUCAACUGGAACCUGACAGAUUU (SEQ ID NO: 12), GCCGUGAUAUUUGGCUUCUUGGCAA (SEQ ID NO: 13), or GGCCCUGAUUGCGUUCAUCAUCUGCAUA (SEQ ID NO: 14), peptide, small molecule (including, but not limited to C29H38O4 (Celastrol), Ellipticine, C54H78N2O17 (Lobophorin F), C19H14O7 (5-Methoxysterigmatocysin), C16H12O7 (Isorhamnetin), C21H26N2O3 (Tabernaemontanin), C27H34O10 (Verrucarin A 9,10-epoxide), C10H12N4O5 (Inosine), C16H12O7, C18H23NO6, C25H33N3O·ClH, C16H12O7 (Rhamnetin), C12H13N5O4 (Toyocamycin), C17H19N3O4 (Anthramycin methyl ether), C21H22N2O3 (Paquinimod), C20H18N2O5·Na (Camptothecin sodium), C18H16O8 (Rosmarinic Acid), C10H14O3, C9H16N2O6 (Tetrahydrouridine), C10H14O3 (Mephenesin), C10H14O3 (4-Ethoxy-3-methoxybenzyl alcohol), C10H14O3 (5-tert-butyl-2-methyl-3-furoic acid), C21H20F3NO6, C23H32N2O6, C15H10N2O, C36H48N2O12 (Rhoodomycin A), C16H24O5 (Ocalicine subst.), C14H25N3O9 (Kasugamycin), C4H6O2 (Crotonic Acid), C21H26N2O3 (Vincamine), Fumitremorgin C, C10H12N4O5 (Formycin B), C18H23NO6 (Swazine), C25H33N3O·ClH (Butylcycloheptylprodiginine Hydrochloride), C18H22N2O4·C6H8O7 (Quinocarcin monocitrate), C23H28N2O5, C11H13NO2·BrH, C21H24N2O3 (Strychninic acid), C21H22N2O3 (Pseudostrychnine), C20H18N2O5·Na (Camptothecin sodium), C18H16O8 (Centaureidin), C10H14O3 (Ramulosin), C19H26N2·C4H6O6, C15H10N2O6 (Lomondomycin), C21H26N2O3 (Pseudoyohimbine), or C26H28O5 (Glycyrrhizol A)), or antibody that binds to CMTM4, inhibits the activity of CMTM4, or inhibits the expression of CMTM4.

In one aspect, disclosed herein are methods of decreasing expression of IL-6 and/or CMTM4 in a tumor microenvironment in a subject, the method comprising administering to the subject C29H38O4 (Celastrol), Ellipticine, C54H78N2O17 (Lobophorin F), C19H14O7 (5-Methoxysterigmatocysin), C16H12O7 (Isorhamnetin), C21H26N2O3 (Tabemaemontanin), C27H34O10 (Verrucarin A 9,10-epoxide), C10H12N4O5 (Inosine), C16H12O7, C18H23NO6, C25H33N3O·ClH, C16H12O7 (Rhamnetin), C12H13N5O4 (Toyocamycin), C17H19N3O4 (Anthramycin methyl ether), C21H22N2O3 (Paquinimod), C20H18N2O5·Na (Camptothecin sodium), C18H16O8 (Rosmarinic Acid), C10H14O3, C9H16N2O6 (Tetrahydrouridine), C10H14O3 (Mephenesin), C10H14O3 (4-Ethoxy-3-methoxybenzyl alcohol), C10H14O3 (5-tert-butyl-2-methyl-3-furoic acid), C21H20F3NO6, C23H32N2O6, C15H10N2O, C36H48N2O12 (Rhoodomycin A), C16H24O5 (Ocalicine subst.), C14H25N3O9 (Kasugamycin), C4H6O2 (Crotonic Acid), C21H26N2O3 (Vincamine), Fumitremorgin C, C10H12N4O5 (Formycin B), C18H23NO6 (Swazine), C25H33N3O·ClH (Butylcycloheptylprodiginine Hydrochloride), C18H22N2O4·C6H8O7 (Quinocarcin monocitrate), C23H28N2O5, C11H13NO2·BrH, C21H24N2O3 (Strychninic acid), C21H22N2O3 (Pseudostrychnine), C20H18N2O5·Na (Camptothecin sodium), C18H16O8 (Centaureidin), C10H14O3 (Ramulosin), C19H26N2·C4H6O6, C15H10N2O6 (Lomondomycin), C21H26N2O3 (Pseudoyohimbine), or C26H28O5 (Glycyrrhizol A) or a siRNA and wherein the siRNA comprises UUAGAUUCCAGUUGAUCUGGG (SEQ ID NO: 1), UGUUAGAUUCCAGUUGAUCUG (SEQ ID NO: 2), ACCAAAUCUGUUAGAUUCCAG (SEQ ID NO: 3), AGAAAACUUGAUUAGAAGGAC (SEQ ID NO: 4), AAGAAAACUUGAUUAGAAGGA (SEQ ID NO: 5), AGAAAGAAAACUUGAUUAGAA (SEQ ID NO: 6), UGAAUUUUUACCAAACAGGAC (SEQ ID NO: 7), AAGUGAAUUUUUACCAAACAG (SEQ ID NO: 8), UUUAUUAAGGUUUUGACUCAU (SEQ ID NO: 9), UAUACUUCCCUUCUCAAUGCC (SEQ ID NO: 10), CTTGATTAGAAGGACGGTT (SEQ ID NO: 11), AGAUCAACUGGAACCUGACAGAUUU (SEQ ID NO: 12), GCCGUGAUAUUUGGCUUCUUGGCAA (SEQ ID NO: 13), or GGCCCUGAUUGCGUUCAUCAUCUGCAUA (SEQ ID NO: 14).

### VI. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows that CMTM4 is highly expressed in multiple cancer types. Gene profiling of multiple murine cancer cell lines including 4T1 breast cancer cells, LLC lung carcinoma cell line, and MCA26 colon cancer cells was performed by chemokine real-time PCR super array.

FIGS. 2A, 2B, 2C, 2D, 2E, 2F, and 2G show that CMTM4 is highly expressed in human carcinoma samples and is a poor prognostic indicator for survival in these patients. FIG. 2A shows CMTM4 expression in human cancer tissues vs. normal tissues from TCGA and GTEx databases. FIG. 2B shows paraffin-embedded human carcinoma sections were stained with anti-CMTM4 antibody (40×). FIG. 2C shows CMTM4 expression in different stages of lung adenocarcinoma was analyzed. FIG. 2D shows CMTM4 expression in various human breast cancer cell lines was determined by real-time PCR. FIGS. 2E and 2F show that lung cancer 2(E, p=0.02) and breast cancer (2F, p=0.0027) patients were divided into high (red) vs. low (green) expression groups based on median expression values of CMTM4. The Kaplan-Meier plot was drawn based on CMTM4 expression level and correlated with overall survival rates. FIG. 2G shows CMTM4 expression in mouse cancer cell lines and normal tissues was assessed by qRT-PCR analysis. All samples were quantitated in triplicate with data representing the mean±SD.

FIGS. 3A, 3B, and 3C show that CMTM4 KD exerts no effect on cell proliferation in vitro. FIGS. 3A and 3B show plasmids containing sequences corresponding to shRNA targeting CMTM4 or control vector were transfected into various cancer cell lines. Inhibition of CMTM4 expression was confirmed by RT-PCR (A) and western blot (3B). FIG. 3C shows tumor cells (10⁴/well) were cultured in 96-well flat plates. After 2 days, proliferation was determined based on [³H]-thymidine uptake.

FIGS. 4A, 4B, 4C, and 4D show that CMTM4 knockdown results in a reduction in tumor growth in vivo. FIG. 4A shows control or CMTM4 KD tumor cells were inoculated into the flanks of BALB/c (4T1 or MCA26 cells) or C57BL/6 (B16 or LLC cells) mice (n=3). Starting at ten days post-tumor implantation, tumor size was measured every 2 days. FIG. 4B shows control or CMTM4 KO LLC and 4T1 cells were inoculated into mice. Tumor growth was measured every 3-4 days. FIG. 4C shows control or CMTM4 KO 4T1 cells were inoculated into mice. Tumor growth was measured every 3-4 days. The weight of tumors is presented. FIG. 4D shows control or CMTM4 KO H292 cells were inoculated into mice. Tumor growth was measured every 3-4 days. The weight of tumors is presented.

FIGS. 5A, 5B, 5C, 5D, 5E, 5F, and 5G shows that CMTM4 knockdown alters leukocyte recruitment. FIGS. 5A, 5B, and 5C shows control or CMTM4 KD LLC cells were inoculated into C57BL/6 mice (n=3). Once tumors reached the size equal to or greater than 1×1 cm², mice with similarly sized tumors were sacrificed and bone marrow, spleen, and tumor tissues were harvested. (C) Cell populations defined by the gating strategy were projected onto t-SNE space and assigned specific colors with CyTOF analysis (upper). MFI of specific gene expression (lower). FIG. 5A shows single-cell suspensions were prepared from each and subjected to FACS analysis. FIG. 5B shows the number of MDSC from the tumor was determined. FIG. 5D shows LLC control or CMTM4 KD cancer cells were inoculated intrahepatically. Immunofluorescent staining of tumor tissues was performed using anti-CD31 and DAPI on LLC control or CMTM4 KD (20×). FIG. 5E shows control or CMTM4 KD LLC cells were inoculated into C57BL/6 mice (n=3). Tumor-infiltrating T cell populations were assessed by FACS analysis (upper) and the number of T cells in the tumor tissues was calculated (lower). FIG. 5F shows MDSCs from bone marrow and spleen of LLC control or CMTM4 KD tumor-bearing mice were cultured with OT-II T cells in the presence of OVA peptides. Proliferation was determined based on [³H]-thymidine uptake. FIG. 5G shows intracellular staining was performed to assess iNOS and arginase 1 (Arg) expression in tumor-infiltrating monocytic MDSCs. *p<0.05, **p<0.01, ***p<0.001, ****p<0.0001. The data shown are representative of three reproducible experiments.

FIGS. 6A, 6B, 6C, 6D, 6E, 6F, 6G, 6H, 6I, 6J, 6K, 6L, and 6M show CMTM4 KD in tumor cells leads to decreased expression and activation of receptor tyrosine kinases. FIG. 6A shows decreased signaling pathways analyzed by IPA in CMTM4 KD LLC cells compared to control LLC cells. FIG. 6B shows a heatmap visualization of normalized DEGs associated with receptor tyrosine kinase (RTK) gene expression by CMTM4 KD in LLC cells from RNAseq analysis. FIG. 6C shows mRNA expression of RTKs was performed by real-time PCR. All samples were quantitated in triplicate with data representing the mean±SD. FIG. 6D shows the phospho-RTK array was performed in control and CMTM4 KD LLC cells and quantitative analysis was performed. FIG. 6E shows EGFR expression by CMTM4 KD in LLC and MCA26 cells were determined by qRT-PCR and western blot. FIG. 6F shows 293T cells were transfected with His-CMTM4 and/or EGFR. Immunoprecipitation with anti-His antibodies was performed, followed by western blot with antibodies against EGFR and CMTM4. FIG. 6G shows Akt/mTOR signaling was checked by western blot in LLC control and CMTM4 KD cells. FIG. 6H shows phosphorylation of NF-κB in control and CMTM4 KD LLC cells was detected by western blot. FIG. 6I shows NF-κB activity was determined in control and CMTM4 KD LLC cells transfected with plasmids carrying TRAF6 dominant-negative mutation (TRAF6 DN), pcDNA-CMTM4, LZM/T6 deleted CMTM4 (CMTM4-LZM/T6), LZM deleted CMTM4 (CMTM4-LZM), or STAT5 deleted CMTM4 (CMTM4-STAT5). FIG. 6J shows heatmap visualization of normalized DEGs associated with receptor tyrosine kinase (RTK)-related gene expression by CMTM4 KD in LLC cells from RPPA. FIG. 6K shows cytokine and chemokine expression in control and CMTM4 KD LLC cells were determined by qRT-PCR. All samples were quantitated in triplicate with data representing the mean±SD. FIG. 6L shows LLC control and CMTM4 KD cells were treated with 10 ng/ml EGF for 2 days. G-CSF production was detected by ELISA. FIG. 6M shows the correlation between CMTM4 and EGFR was analyzed from human cancer patient data. *p<0.05, **p<0.01, ***p<0.001.

FIGS. 7A and 7B show that CMTM4 regulates EGFR expression. EGFR expression by CMTM4 KD/KO in multiple cancer cells was determined by qRT-PCR.

FIGS. 8A, 8B, 8C, 8D, 8E, 8F, 8G, 8H, 8I, 8J, 8K, 8L, 8M, 8N shows CMTM4 regulates the reorganization of lipid raft to control EGFR expression. FIG. 8A shows fractions from lipid raft and non-raft regions were isolated and western blot was performed for CMTM4. Flotillin-1 was used as a marker of lipid raft and vinculin was used as a maker of the non-raft fraction. FIG. 8B shows control and CMTM4 KD LLC cells were treated with EGF for 1 hour and expression of lipid raft was stained with CT-B. FIG. 8C shows heatmap visualization of normalized DEGs associated with lipogenesis, cholesterol efflux, and signal transduction gene expression by CMTM4 KD in LLC cells from RNAseq analysis. FIGS. 8D and 8E show LLC CMTM4 KD cells were treated with MβCD and real-time PCR (8D) and western blot (8E) were performed to determine EGFR levels. *p<0.05. FIG. 8F show 293T cells were transfected with CMTM4 WT-mcherry (upper) or CMTM4 MT-mcherry (lower) with EGFR-EGFP and then, cells were treated with EGF for 1 hour. Representative confocal images are shown. FIG. 8G show RNAseq data showing Rab expression between control and CMTM4 KD LLC cells. FIGS. 8H and 8I show Rab expression in control and CMTM4 KD LLC cells were determined by qRT-PCR (H) and western blot (8I). FIGS. 8J, 8K, and 8L show 293T cells were transfected with CMTM4-EGFP and Rab4 (mCherry), Rab5 (mCherry), or Rab11 (mCherry), and then, cells were treated with EGF for 1 hour. Representative confocal images are shown. Pearson correlation coefficient (PCC) was measured from 20 individual cells. FIGS. 8M and 8N show EGFR-EGFR and Lamp-1 RFP were transfected to LLC control and CMTM4 KD cells. Confocal imaging was taken before (8M) and after (8N) treatment with EGF. Pearson correlation coefficient (PCC) was measured from 20 individual cells. *p<0.05, **p<0.01, ***P<0.001, ****P<0.0001. Scale bar: 3 μm.

FIGS. 9A, 9B, and 9C show CMTM4 siRNA liposomes reduce CMTM4 expression (9A) Control or CMTM4 siRNAs were transfected to LLC cells and CMTM4 levels were determined by qRT-PCR. FIG. 9B shows control or CMTM4 siRNA liposomes were treated to LLC cells and CMTM4 levels were determined by qRT-PCR. FIG. 9C shows control or CMTM4 siRNA liposomes were treated to macrophages and CMTM4 levels were determined by qRT-PCR.

FIG. 10 shows CMTM4 siRNA liposomes inhibits tumor growth in vivo LLC cells were injected to mice subcutaneously. After 7 days, 30 ug control or CMTM4 siRNA liposomes were injected twice per week.

FIGS. 11A, 11B, and 11C show CMTM4 siRNA liposomes prolong mouse survival and reduce lung metastasis. 1×10₅ 4T1 cells were injected to mice intravenously. From day 4, 30 ug control or CMTM4 siRNA liposomes were injected to mice intravenously every three days. FIG. 11A shows mouse survival was determined. FIG. 11B shows lung weights were measured. FIG. 11C shows lung pictures. ***p<0.001.

FIG. 12 shows CMTM4 siRNA liposomes have synergistic effect with T cells. Mice were injected 3×10₄ 4T1 cells intravenously. From day 4, 30 ug control or CMTM4 siRNA liposomes were injected to mice intravenously twice every week. After 8 days of tumor implantation, 5×10₆ DO11 T cells or PBS were transferred to mice. *p<0.05, **p<0.01, ***P<0.001

FIG. 13 shows screening of compounds targeting CMTM4

FIGS. 14A, 14B, 14C, 14D, and 14E show that CMTM4 is involved in macrophage polarization. FIG. 14A shows mouse bone marrow cells were cultured with 50 ng/ml M-CSF or 50 ng/ml GM-CSF for 4 days and then, CMTM4 expression was determined by real-time PCR. FIGS. 14B and 14C show mouse bone marrow cells were treated with 50 ng/ml M-CSF (14B) or 50 ng/ml GM-CSF (14C) for indicated time points. CMTM4 expression was determined by real-time PCR. FIG. 14D shows mouse bone marrow cells were induced to differentiate to macrophages with 50 ng/ml M-CSF for 4 days. Cells were treated with 20 ng/ml IFNγ and 10 ng/ml LPS for 8 hours. CMTM4 expression was determined by real-time PCR. FIG. 14E shows that microarray data were downloaded from expression atlas and CMTM4 expression was analyzed in human monocytes treated with LPS, IL-10, IFNγ, TNF-α, and S100A8.

FIGS. 15A, 15B, 15C, 15D, 15E, 15F, 15G, 15H, 15I, and 15J shows that CMTM4 KO promotes M1-like macrophage polarization. Bone marrow cells from CMTM4F/r and CMTM4_{F/F}LysM_{cre} mice were cultured with M-CSF for 4 days and then, the macrophages were stimulated with 20 ng/ml IFNγ and 10 ng/ml LPS for indicated time points. Gene expression was determined by real-time PCR. GAPDH was used as an internal control. FIGS. 15D, 15E, 15F, 15G, 15H, and 15I show mouse bone marrow cells were induced differentiation to macrophages with 50 ng/ml M-CSF for 4 days. Cells were treated with 20 ng/ml IL-4, 10 ng/ml LPS, or 50 ng/ml GM-CSF for 2 hours. Gene expression was determined by real-time PCR. FIG. 15J show M-CSF-induced macrophages from CMTM4F/F and CMTM4_{F/F}LysM_{cre} mice were stimulated with 20 ng/ml IFNγ and 10 ng/ml LPS for 2 hours. Cytokine production was detected by ELISA.

FIGS. 16A, 16B, and 16C show that CMTM4 KO enhances M1 signaling activation whereas decreased M2 signaling activation. FIGS. 16A and 16B show M-CSF-induced macrophages from CMTM4_{F/F} and CMTM4_{F/F}LysM_{cre} mice were stimulated with 20 ng/ml IFNγ, 10 ng/ml LPS, 20 ng/ml IL-4, or 20 ng/ml IL-10 for indicated times. Proteins were detected by western blot. FIG. 16C shows bone marrow cells from CMTM4F/F and

FIGS. 17A, 17B, 17C, 17D, 17E, 17F, 17G, 17H, and 17I show that CMTM4 KO alters macrophage transcriptome. FIGS. 17A and 17B show volcano plots of normalized gene enrichment p-values of macrophages differentiated from CMTM4_{F/F} and CMTM4_{F/F}LysM_{cre} mice in M1 (17A) and M2 (17B) conditions. DEGs meeting statistical significance and above enrichment cutoff (DEGs with FC≥1.5 and p-value<0.01 with FDR) are shown in green. FIG. 17C shows a heatmap visualization of normalized DEGs associated with top 100 increased 51 gene expression by CMTM4 KO in M1 condition. Data is normalized by row z-score. FIG. 17D shows gene ontology (GO) enrichment analysis of genes differentially expressed in macrophages from CMTM4F/F and CMTM4_{F/F}LysM_{cre} mice in M1 condition. FIGS. 17E and 17G shows a heatmap visualization of normalized DEGs associated with top 100 decreased (green) gene expression by CMTM4 KO in M1 condition (17E) and DEGs associated with gene expression by CMTM4 KO in M2 condition (17G). FIGS. 17F and 17H show gene ontology (GO) enrichment analysis of genes differentially expressed in macrophages from CMTM4_{F/F} and CMTM4_{F/F}LysM_{cre} mice in M1 condition (17F) and in M2 condition (17H). FIG. 17I shows increased signaling pathways analyzed by Ingenuity pathway analysis (IPA) in macrophages from CMTM4_{F/F}LysM_{cre} mice compared to CMTM4_{F/F}mice in M1 condition. *p<0.05, **p<0.01, ***p<0.001. Data shown are representative of three reproducible experiments.

FIGS. 18A and 18B show expected disease and disorders (18A) and physiological system development and function changes (18B) by CMTM4 KO in macrophages analyzed by IPA

FIGS. 19A, 19B, 19C, 19D, 19E, 19F, 19G, 19H, 19I, 19J, and 19K show that myeloid CMTM4 KO increases susceptibility to DSS-induced colon inflammation. FIG. 19A shows a schematic representation of the DSS protocol. FIG. 19B shows bodyweight changes of the mice. Data are means±SEM (n=13) FIG. 19C shows representative images of colons (upper) and average colon length (lower) from CMTM4^{F/F} and CMTM4^{F/F}LysM^{cre} mice. n=11-13. FIG. 19D show myeloid cells from lamina propria were determined by FACS analysis. FIG. 19E shows cell populations defined by the gating strategy were projected onto t-SNE space and assigned specific colors with CyTOF analysis of DSS treated mice. FIG. 19F show t-SNE map, percentage, cell number of neutrophils from CyTOF analysis. FIG. 19G shows cluster heat map demonstrating expression levels of myeloid cell and functional markers used as the embedding parameters. FIG. 19H shows colon lysates were prepared from normal and DSS-treated mice and were analyzed by western blotting (one mouse per lane) with the indicated antibodies. FIG. 19I shows volcano plot representing the RNA-seq results. FIG. 19J shows host significantly changed genes in RNA-seq datasets. FIG. 19K shows increased signaling pathways analyzed by IPA in lamina propria macrophages from CMTM4^{F/F}LysM^{cre} mice compared to CMTM4^{F/F} mice. *p<0.05, **<0.01, ****p<0.0001.

FIGS. 20A, 20B, 20C, 20D, 20E, 20F, 20G, 20H, and 20I show that myeloid CMTM4 KO mice are more susceptible to AOM/DSS-driven colon tumorigenesis. FIG. 20A shows a schematic of the AOM/DSS-driven colon tumorigenesis model. CMTM4^{F/F} and CMTM4^{F/F}LysM^{cre} mice received a single injection of AOM one day prior to DSS administration. The mice were then treated with three rounds of 2% DSS for 5 days; the treatment was followed by 2 weeks of recovery. FIG. 20B shows weight loss was monitored through the AOM/DSS model. FIG. 20C shows polyps were identified in the distal and mid colons and counted by the sizes. FIG. 20D shows composite clinical score reflecting the combination of weight loss, stool consistency, and the presence of blood in the stool and/or rectum. FIG. 20E shows cytokine levels from serum were determined by ELISA. FIG. 20F shows colon sections were stained with H&E and histologically analyzed. Scale bar, 100 μm. FIGS. 20G and 20H show colon sections were stained with Ki-67 and Ki-67+ cells were determined by Image J (20H). Scale bar, 100 μm. FIG. 20I show immune cells were isolated from lamina propria. The cells were analyzed by FACS. *p<0.05, **p<0.01, ***p<0.001, ****p<0.0001.

FIGS. 21A, 21B, 21C, 21D, 21E, 21F, 21G, 21H, 21I, 21J, 21K, 21L, and 21M shows that myeloid CMTM4 KO mice suppress tumor progression in genetically engineered and transplanted tumor models. FIGS. 21A, 21B, 21C, 21D, and 21E show bone marrow cells from CMTM4^{F/F} and CMTM4^{F/F}LysM^{cre} re mice were transferred to APC^{min/+} mice. After 110-120 days, tumor formation was determined. FIGS. 21A and 21B show polyp numbers from the colon (21A) and small intestine (21B) were determined. n=9. FIGS. 21C and 21E show tissue sections from colon and small intestine were stained with Ki-67 and Ki-67+ cells were determined by Image J. FIG. 21D show colon and small intestine sections were stained with H&E and histologically analyzed. FIG. 21F shows bone marrow cells from CMTM4F/F and CMTM4^{F/F}LysM^{cre} mice were transferred to MMTV-PyMT mice. After 90-100 days, tumor formation was determined. n=9. FIGS. 21G, 21H, 21I, 21K, and 21L show LLC cells were inoculated into from CMTM4^{F/F} and CMTM4^{F/F}LysM^{cre} mice. Data were combined from three independent experiments (n=3-4 per group). FIG. 21G shows tumor growth was measured every 3 days. FIG. 21H shows tumor weight was determined at day 27 post-inoculation of the cells. FIGS. 21I, 21J, and 21K show immune cells were isolated from tumors and analyzed by FACS. FIG. 21L shows T cells were purified from spleen and stimulated with α-CD3 and α-CD28 for 2 days. Cytokine concentration in supernatants was measured by ELISA. FIG. 21M shows LLC cells were inoculated intrahepatically into CMTM4^{F/F} or CMTM4^{F/F}LysM^{cre} mice. Immunofluorescent staining was performed on tumor tissue sections using anti-CD31 and DAPI (20× magnification). *p<0.05, **p<0.01, ***p<0.001.

FIGS. 22A, 22B, 22C, 22D, 22E, 22F, and 22G show that CMTM4 KO macrophages have increased M2-dependent metabolic pathways in an inflammatory condition. FIG. 22A shows single-cell RNAseq (scRNAseq) analysis was performed with LLC-tumor infiltrated leukocytes and lamina propria leukocytes from DSS-treated mice. FIG. 22B shows a heatmap overview of gene expression of each cluster from scRNAseq analysis. (FIG. 22C shows increased and decreased signaling pathways analyzed by IPA in lamina propria neutrophils vs. tumor-infiltrating neutrophils from CMTM4^{F/F}LysM^{cre} mice. FIG. 22D shows a heatmap of gene expression related to M2-dependent metabolic pathways between tumor-infiltrating macrophages and lamina propria macrophages. FIG. 22E shows a schematic of neutrophil depletion in the AOM/DSS-driven colon tumorigenesis model. CMTM4^{F/F} and CMTM4^{F/F}LysM^{cre} mice were injected with control IgG or a-Ly6G antibody every three days before and during DSS treatment. The mice received a single injection of AOM one day prior to DSS administration. The mice were then treated with three rounds of 2% DSS for 5 days; the treatment was followed by 2 weeks of recovery. FIGS. 22F and 22G show that polyps were identified in the distal and mid colons (22F) and counted the numbers (22G).

FIGS. 23A, 23B, 23C, 23D, 23E, 23F, and 23G show that neutrophil depletion recovers DSS-mediated inflammation and reprograms M1 macrophages in myeloid CMTM4 KO mice (23A, 23B, 23C, 23D, 23E, and 23F) CMTM4^{F/F} and CMTM4^{F/F}LysM^{cre} mice were injected control IgG or a-Ly6G antibody every three days from one day prior to DSS administration. FIG. 23A shows bodyweight changes of the mice. Data are means±SD, FIG. 23B shows the average colon length from CMTM4^{F/F} and CMTM4^{F/F}LysM^{cre} mice. FIG. 23C shows neutrophil depletion was determined by FACS analysis. FIG. 23D shows a volcano plot representing the RNA-seq results of lamina propria macrophages between a-Ly6G-treated and control IgG-treated CMTM4^{F/F}LysM^{cre} mice. FIG. 23E shows the most significantly changed macrophage function-related genes in RNA-seq datasets. FIGS. 23F and 23G shows a heatmap of inflammatory chemokines and cytokines (23F) and oxidative phosphorylation pathway-related genes (23G) in lamina propria macrophages from control IgG- and a-Ly6G-treated myeloid CMTM4 KO mice. *p<0.05.

FIGS. 24A and 24B show that CMTM4 KD tumors reduce infiltration of PMN-MDSC FIGS. 24A and 24B show MaFIA mice were inoculated intrahepatically with LLC control or CMTM4 KD tumors (n=3). MDSCs were harvested from CD45.1 LLC tumor-bearing mice and sorted into PMN and monocytic populations. When tumors reached 7×7 to 9×9 mm² CD115⁺ cells were depleted. On the same day, sorted PMN-MDSC and M-MDSCs were injected i.v. into CD115-depleted MaFIA tumor-bearing mice. After recipient mice were terminated, the numbers of tumor-infiltrating donor (CD45.1) MDSCs were determined. *p<0.05, ***p<0.001. The data shown are representative of three reproducible experiments.

FIG. 25 shows the percentage of immune cells determined by CyTOF analysis. CMTM4^{F/F} and CMTM4^{F/F}LysM^{cre} mice were treated with 2% DSS for 5 days. Lamina propria immune cells were isolated on day 10 and immune cell profiles were determined by CyTOF analysis.

FIG. 26 shows the survival rate of mice during AOM/DSS-driven colon tumorigenesis.

FIGS. 27A, 27B, 27C, and 27D show immune cell profiles from AOM/DSS-induced colorectal cancer mice. CMTM4^{F/F} and CMTM4^{F/F}LysM^{cre} mice received a single injection of AOM one day prior to DSS administration. The mice were then treated with three rounds of 2% DSS for 5 days; the treatment was followed by 2 weeks of recovery. Immune cells were isolated from lamina propria (27A and 27C) and tumors (27B and 27D) in colons and analyzed by FACS.

FIGS. 28A, 28B, and 28C show western blots of a 4T1 tumor model revealing that vinculin expression was significantly increased in CMTM4 knockout mice when treated with C10H14O3 (4-Ethoxy-3-methoxybenzyl alcohol)(28A), C9H16N2O6 (Tetrahydrouridine)(28B), or C10H14O3 (5-tert-butyl-2-methyl-3-furoic acid)(28C). 1 mm of C10H14O3 (4-Ethoxy-3-methoxybenzyl alcohol)(28A), C9H16N2O6 (Tetrahydrouridine)(28B), or C10H14O3 (5-tert-butyl-2-methyl-3-furoic acid)(28C) was administered to control mice or CMTM4 knockouts and expression measured after 0, 3, 6, and 24 hours.

FIGS. 29A, 29B, 29C, 29D, and 29E show CMTM4 involve in regulation of IL-6 responses, FIG. 29A shows that cytokine IL-6 levels from blood were determined by ELISA in CMTM4F/FLysMcre and CMTM4F/FLysMcre control mice. FIG. 29B shows cytokine IL-6 levels from blood were determined by ELISA in HCC827-CT and HCC827-CMTM4 KO mice. FIG. 29C shows a diagram of the transmembrane domains of CMTM4 (CMTM4 full length, CMTM4 M1, CMTM4 M2, CMTM4 M3, CMTM4 M4, CMTM4 M5 and CMTM4 Ser to Ala) plasmid. FIG. 29D shows that CMTM4 protein expression in transduced HCC827-CMTM4-KO) cells was verified by IP. FIG. 29E shows that cytokine IL-6 levels in HCC827-CT and HCC827-CMTM4-KO transduced cells.

FIGS. 30A, 30B, 30C, 30D, 30E, and 30F show that CMTM4 deletion decreases ER stress gene expression in M1, M2 macrophage. FIGS. 30A and 30B show the expression of ER stress related proteins (p-IRE-1, p-EIF2, ATF4, XBP-1s, CHOP, PERK) in bone marrow derived M0, M1 and M2 macrophages. FIG. 30C shows the phenotype of BMDM from WT and CMTM4f/f—LysMcre mice were assessed by FACS analysis: M1 Macrophages (CD45+CD11b+F4/80High CD86+). M2 Macrophages (CD45+CD11b+F4/80High CD206+). FIG. 30D shows BODIPY staining for lipid content in CMTM4f/f-LysMcre and WT M1 and M2 macrophages. FIGS. 30E and 30F show co-immunoprecipitation (Co-IP) of CMTM4 and TRAF6 in HCC827-CT cell and association of CMTM4 with TRAF6 and TRAF2 by co-IP. Co-IP of CMTM6 and TRAF6 in HCC827-CT cells. The association of CMTM6 with TRAF6 was not detected.

FIGS. 31A, 31B, 31C, 31D, 31E, 31F, 31G, and 31H show that CMTM4 interacts with TRAF2 and RAB35/21 proteins by FRED assay. FIGS. 31A and 31B show the FRET assay was used to identify the CMTM4 associated protein. FIG. 31C shows MHC II level of 4T1 gRNA control or CMTM4 KO cells treated with IFNγ and chloroquine (CQ). Unpaired t test. **: p<⁰0.01. FIG. 31D shows mass-spectrometry of CMTM4 associated proteins. FIG. 31E shows immunoprecipitation of CMTM4 and blotted by Rab35. FIG. 31F shows 4T1 gRNA control or CMTM4 KO cells were treated with IFN and fixed to stain for Rab35 (green), LAMP1(red) and MHC II (magenta). Ten fields of each condition were imaged by Nikon A1 confocal and Pearson co-localization was calculated by NIS elements AR analysis software using two fluorescent signals on each image. FIG. 31G shows pearson co-localization scores from ten pooled images pooled for each condition. Yellow arrow: co-localization of Rab35 and LAMP1. Experiment is repeated twice with similar result. Scale bar: 10 μm. Un-paired student t test. *: p<0.05. ***: p<0.001. FIG. 31H shows MHC II levels of 4T1 gRNA control or CMTM4 KO cells transfected with EGFP vector, dominant negative Rab35 (N1201) and constitutive active form of Rab35 (Q67L). Experiments were repeated twice with similar results. Unpaired t test. *: p<0.05. **: p<0.01.

FIGS. 32A, 32B, 32C, 32D, 32E, and 32F show CMTM4 KO can up-regulate MHC Class II and facilitate the TCR T cell therapy. FIG. 32A shows CMTM4 KO human lung cancer cell H292 was treated with hIFN-gamma and the level of human MHC II were quantified. Experiments were repeated twice with similar results. Paired t test. *: p<0.05. FIG. 32B shows CMTM4 KO human lung cancer cell H1437, H2170 and (32C) FM-56 were treated with hIFN-gamma and the level of human MHC II were quantified. FIG. 32D shows subcutaneous tumor growth of 4T1 gRNA and CMTM4 KO in immune-competent mice or in immune-deficient mice. Experiments were repeated twice with similar results. Two-way ANOVA. **: p<0.01. ****: p<0.0001. FIG. 32E shows survival of animal i.v. implanted with 4T1 OVA tumor, treated with CMTM4 siRNA-liposome complex and adoptive transfer of DO 11 T cells. Log-rank (Mantel-Cox) test. *: p<0.05. ***: p<0.001. ****: p<0.0001. FIG. 32F shows the Tumor infiltrating leukocyte subsets from tumor or tumor draining lymph nodes were stained multiple markers followed by analysis with CyTOF.

FIGS. 33A and 33B show compound screening by assessing the effect on IL-6 secretion and CMTM4 expression. FIG. 33A shows IL-6 levels in HCC827-CT cells treated with small compounds at 1 mm concentration for 24 hrs. FIG. 33B shows Western blot of CMTM4 protein in HCC827 CT cells treated with small compounds at 1 μM concentration.

FIGS. 34A, 34B, and 34C show that CMTM4 regulates Akt/mTOR signaling in human cancer, which is more sensitive to EGFR inhibition when CMTM4 is knockout. FIG. 34A shows that EGFR mutated (over activating) cell line HCC827 was knockout of CMTM4 by CRISPR and Cas9 mRNA or protein. Akt/mTOR signaling was checked by western blot. FIG. 34B shows that HCC827 control and CMTM4 KO cells were treated with various concentration of Gefitinib and tumor cell growth was measured by Incucyte. P value calculated by Two-way ANOVA test. FIG. 34C shows HCC827 control and CMTM4 KO cells were treated with 2 M Gefitinib and tumor cell growth was measured by Incucyte. P value calculated by Two-way ANOVA test.

### VII. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular agent that inhibits CMTM4 is disclosed and discussed and a number of modifications that can be made to a number of molecules including the agent that inhibits CMTM4 are discussed, specifically contemplated is each and every combination and permutation of agent that inhibits CMTM4 and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Inflammation has been recognized as a hallmark of cancer and linked to tumor initiation and progression. Tumor-associated inflammation has also been shown to promote angiogenesis, metastasis, and resistance to chemotherapy, and to subvert immune surveillance. Multiple players are at work within the tumor microenvironment and the exact composition differs depending on various factors, including cancer type, stage of the disease, and host immune status. Therefore, dampening both tumor intrinsic inflammatory signaling and host cell-mediated inflammation via manipulation of a master regulator is a particularly promising approach for developing new cancer therapies.

Chemokine-like factor (CKLF)-like MARVEL transmembrane domain-containing family 4 (CMTM4) belongs to the CMTM family consisting of nine members, CKLF and CMTM1-8. Among CMTM family members, CMTM4 is the most conserved member and has functions in tumor progression and tumor microenvironment. Despite being discovered many years ago, the actual function of CMTM4 remains minimally characterized. Interestingly, while CMTM4 is expressed in low and variable amounts in multiple normal human tissues, it is universally expressed in a multitude of human cancers. Recently, CMTM4 has been shown to regulate PD-L1 expression. To better define the function of CMTM4, as it relates to tumor-associated inflammation and tumor progression, we investigated its expression profile in human cancer patients and mouse cancer cells and assessed the effects of CMTM4 on tumor growth and tumor-related inflammation. In the current study, we identified the role of CMTM4 and its related signaling pathway in the regulation of tumor-associated inflammation, tumor progression, and establishment of the suppressive tumor microenvironment.

Chemokine-like factor (CKLF)-like MARVEL transmembrane domain-containing family 4 (CMTM4) belongs to the CMTM family consisting of nine members, CKLF and CMTM1-8. Among CMTM family members, CMTM4 is the most conserved member and has functions in tumor progression and tumor microenvironment. Despite being discovered many years ago, the actual function of CMTM4 remains minimally characterized. Interestingly, while CMTM4 showed low and variable expression in multiple normal human tissues, it was shown to be universally expressed in a multitude of human cancers. Recently, CMTM4 has been shown to regulate PD-L1 expression. Here, we are the first to identify that CMTM4 can be as a novel regulator of macrophage polarization and its opposite functions in inflammatory vs. genetically engineered or transplant tumor models which can modulate antitumor immunity dependent on the tumor microenvironment through the cross regulation of neutrophil and macrophages on the lipid metabolism and inflammation signaling.

As shown herein, a decrease in CMTM4 correlates with an increase the polarization of macrophage towards M1-like macrophage and away from M2-like macrophage; wherein an increase in CMTM4 indicates polarization towards M2-like macrophage. This is significant as M2-like macrophage are associated with metastasis and cell growth. Accordingly, in one aspect, disclosed herein are methods of detecting a cancer (such as, for example, cholangiocarcinoma (CHOL), colon adenocarcinoma (COAD), esophageal carcinoma (ESCA), kidney chromophobe (KICH), pheochromocytoma and paraganglioma (PCPG), prostate adenocarcinoma (PRAD), rectum adenocarcinoma (READ), and thymoma (THYM), breast cancer, colon cancer, melanoma, prostate cancer, a glioma, kidney cancer, or lung adenocarcinoma) in a subject comprising obtaining a tissue sample from the subject and measuring the expression level of Chemokine-like factor (CKLF)-like MARVEL transmembrane domain-containing member 4 (CMTM4) relative to a control, wherein an increase in the expression of CMTM4 relative to the control indicates the presence of a cancer.

Also disclosed herein are methods of assessing the aggressiveness/severity of a cancer (such as, for example, cholangiocarcinoma (CHOL), colon adenocarcinoma (COAD), esophageal carcinoma (ESCA), kidney chromophobe (KICH), pheochromocytoma and paraganglioma (PCPG), prostate adenocarcinoma (PRAD), rectum adenocarcinoma (READ), and thymoma (THYM), breast cancer, colon cancer, melanoma, prostate cancer, a glioma, kidney cancer, or lung adenocarcinoma) and/or whether a cancer in a subject is metastatic comprising obtaining a cancerous tissue sample from a tumor microenvironment in the subject and measuring the expression level of Chemokine-like factor (CKLF)-like MARVEL transmembrane domain-containing member 4 (CMTM4) in the tissue sample relative to a control, wherein an increase in the expression level of CMTM4 relative to the control indicates the cancer is metastatic.

In one aspect, disclosed herein are methods of detecting a cancer of any preceding aspect, methods of assessing the aggressiveness/severity of a cancer of any preceding aspect, or whether a cancer in a subject is metastatic of any preceding aspect; wherein a cancer is detected, or a cancer is found to be metastatic, the method further comprises administering to the subject an agent that inhibits CMTM4. For example, the method can comprise administering a miRNA, shRNA, siRNA (such as, for example, UUAGAUUCCAGUUGAUCUGGG (SEQ ID NO: 1), UGUUAGAUUCCAGUUGAUCUG (SEQ ID NO: 2), ACCAAAUCUGUUAGAUUCCAG (SEQ ID NO: 3), AGAAAACUUGAUUAGAAGGAC (SEQ ID NO: 4), AAGAAAACUUGAUUAGAAGGA (SEQ ID NO: 5), AGAAAGAAAACUUGAUUAGAA (SEQ ID NO: 6), UGAAUUUUUACCAAACAGGAC (SEQ ID NO: 7), AAGUGAAUUUUUACCAAACAG (SEQ ID NO: 8), UUUAUUAAGGUUUUGACUCAU (SEQ ID NO: 9), UAUACUUCCCUUCUCAAUGCC (SEQ ID NO: 10), CTTGATTAGAAGGACGGTT (SEQ ID NO: 11), AGAUCAACUGGAACCUGACAGAUUU (SEQ ID NO: 12), GCCGUGAUAUUUGGCUUCUUGGCAA (SEQ ID NO: 13), or GGCCCUGAUUGCGUUCAUCAUCUGCAUA (SEQ ID NO: 14), peptide, small molecule)), or antibody that binds to CMTM4, inhibits the activity of CMTM4, or inhibits the expression of CMTM4. Examples of small molecules that inhibit CMTM4 are shown herein and include, but not limited to the following:

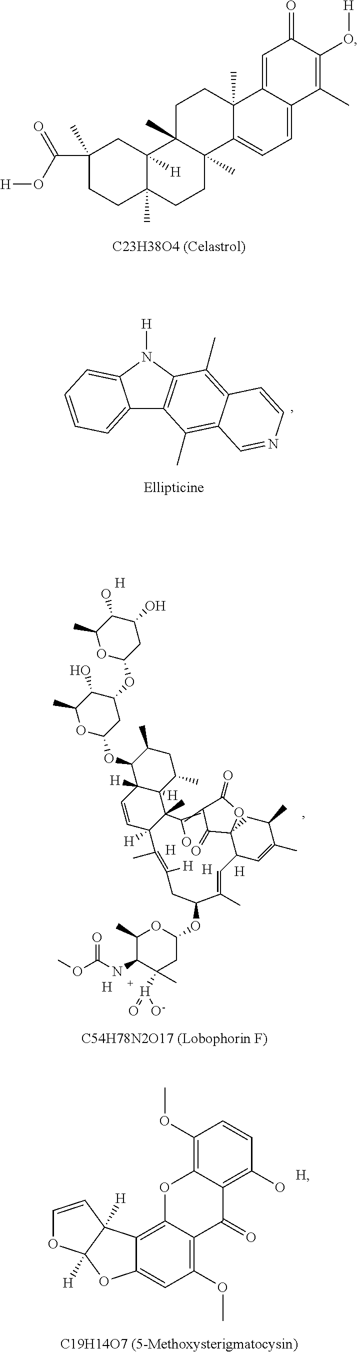

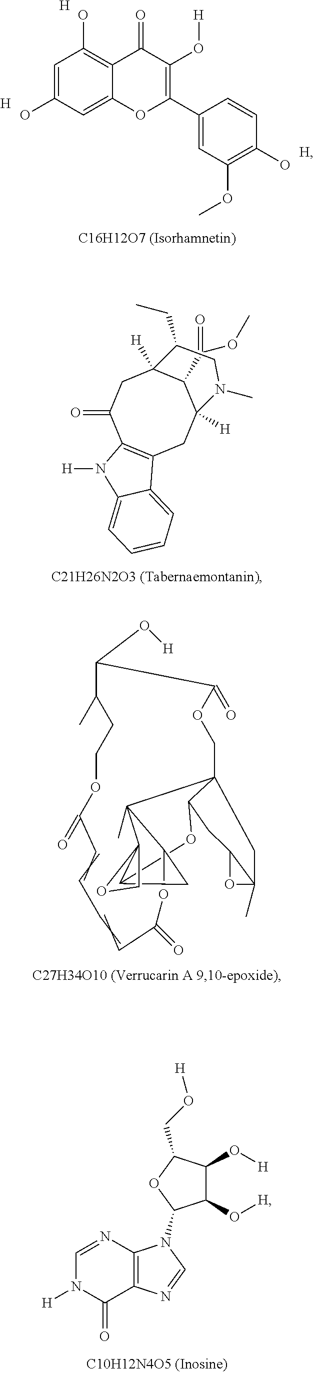

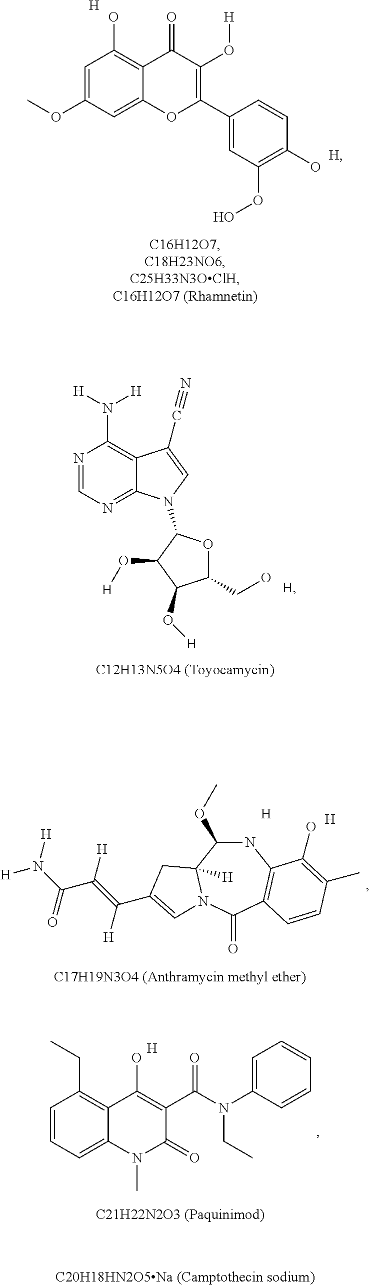

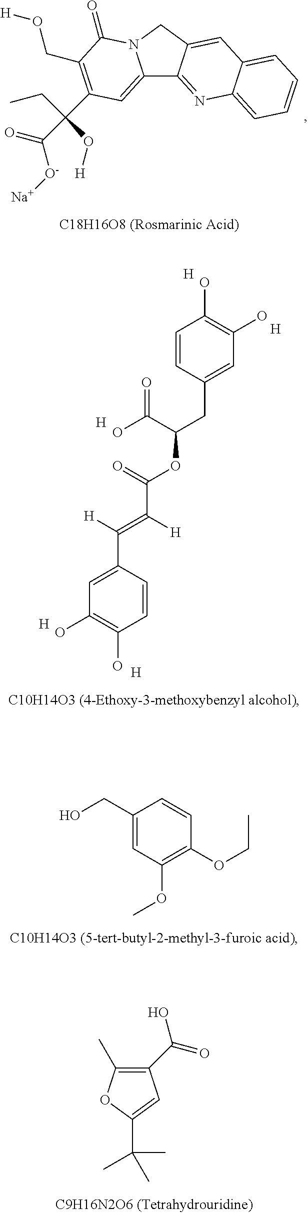

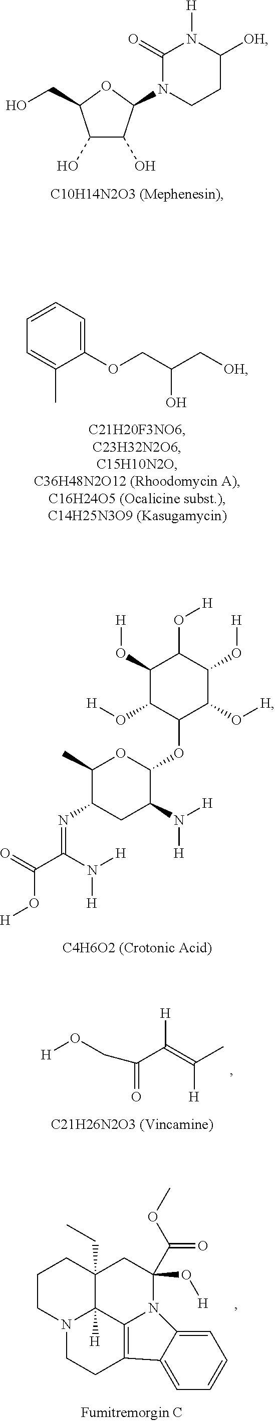

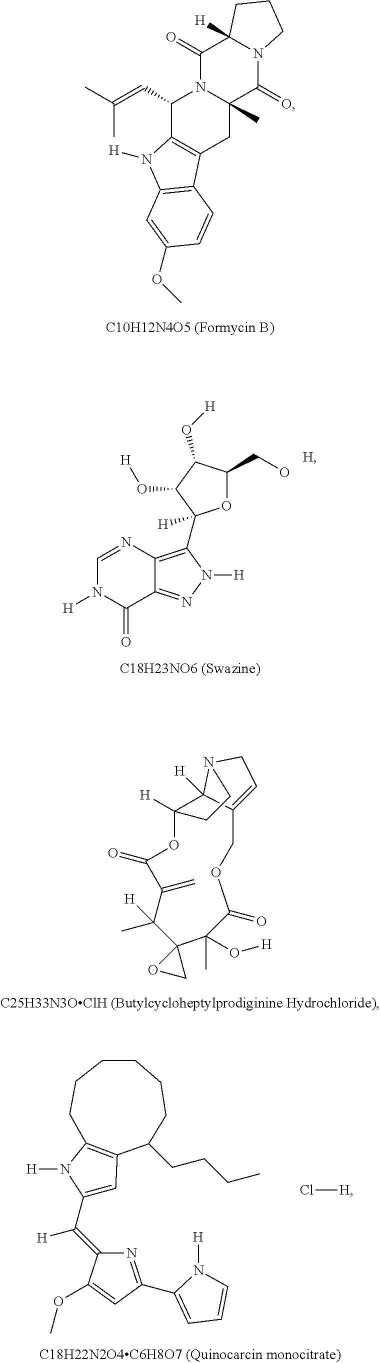

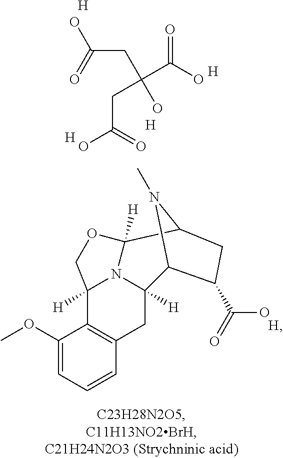

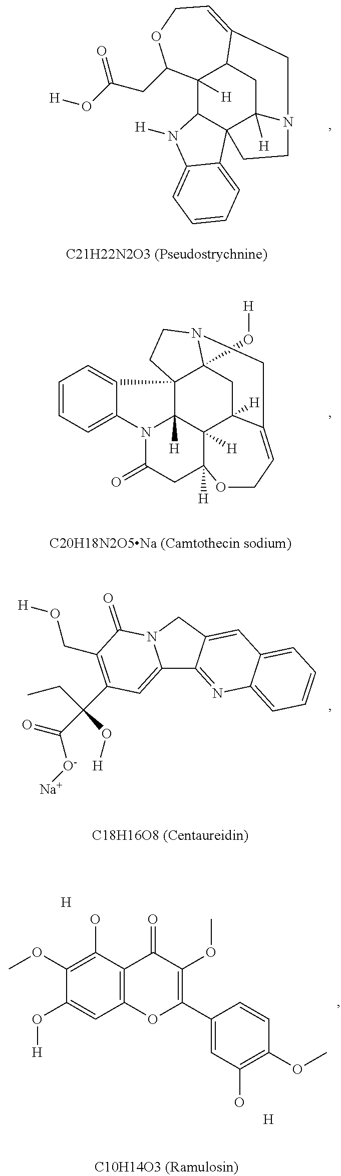

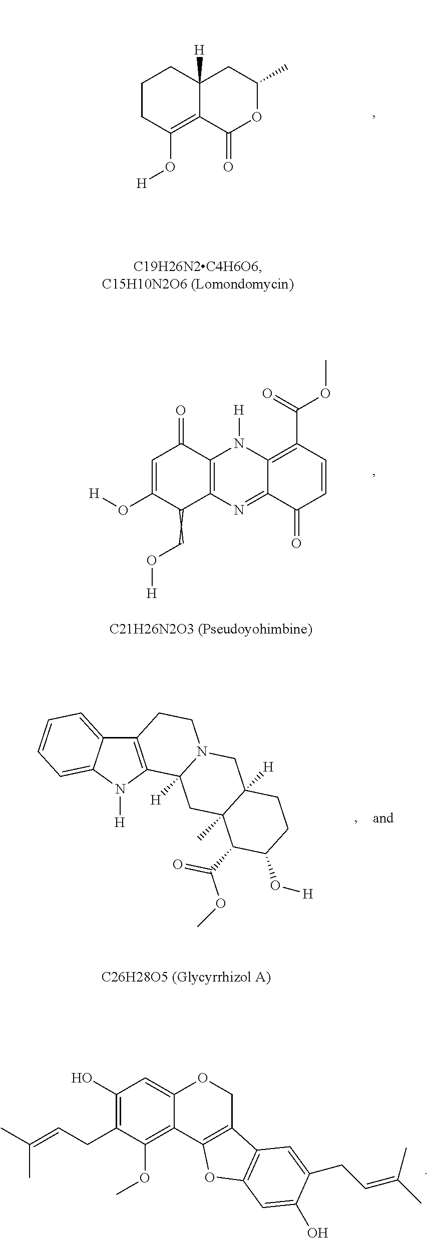 [SMILES: C[C@H]1C[C@H]2CCCC(O)=C2C(=O)O1 ; COC(=O)c1ccc(=O)c2nc3c(=CO)c(O)cc(=O)c3[nH]c12 ; COC(=O)[C@H]1[C@@H](O)CC[C@@H]2CN3CCc4c([nH]c5ccccc45)[C@@H]3C[C@@]21C ; COc1c(CC=C(C)C)c(O)cc2c1-c1oc3cc(O)c(CC=C(C)C)cc3c1CO2]

### C. Method of Treating Cancer

As noted herein, the disclosed CMTM4 inhibitory agents can be used to treat any disease where uncontrolled cellular proliferation occurs such as cancers. A representative but non-limiting list of cancers that the disclosed compositions can be used to treat is the following: lymphomas such as B cell lymphoma and T cell lymphoma; mycosis fungoides; Hodgkin's Disease; myeloid leukemia (including, but not limited to acute myeloid leukemia (AML) and/or chronic myeloid leukemia (CML)); bladder cancer; brain cancer; nervous system cancer; head and neck cancer; squamous cell carcinoma of head and neck; renal cancer (i.e., kidney cancer); lung cancers such as small cell lung cancer, non-small cell lung carcinoma (NSCLC), lung squamous cell carcinoma (LUSC), and Lung Adenocarcinomas (LUAD); neuroblastoma/glioblastoma; ovarian cancer; pancreatic cancer; prostate cancer (including, but not limited to prostate adenocarcinoma (PRAD)); skin cancer; hepatic cancer; melanoma; squamous cell carcinomas of the mouth, throat, larynx, and lung; cervical cancer; cervical carcinoma; breast cancer (including, but not limited to triple negative breast cancer); genitourinary cancer; pulmonary cancer; esophageal carcinoma; head and neck carcinoma; large bowel cancer; hematopoietic cancers; testicular cancer; cholangiocarcinoma (CHOL), colon adenocarcinoma (COAD), esophageal carcinoma (ESCA), kidney chromophobe (KICH), pheochromocytoma and paraganglioma (PCPG), rectum adenocarcinoma (READ), and thymoma (THYM), lung adenocarcinoma, and colon and rectal cancers.

In one aspect, the treatment of the cancer can include administering to the subject an agent that inhibits CMTM4. For example, the method can comprise administering a miRNA, shRNA, siRNA (such as, for example, UUAGAUUCCAGUUGAUCUGGG (SEQ ID NO: 1), UGUUAGAUUCCAGUUGAUCUG (SEQ ID NO: 2), ACCAAAUCUGUUAGAUUCCAG (SEQ ID NO: 3), AGAAAACUUGAUUAGAAGGAC (SEQ ID NO: 4), AAGAAAACUUGAUUAGAAGGA (SEQ ID NO: 5), AGAAAGAAAACUUGAUUAGAA (SEQ ID NO: 6), UGAAUUUUUACCAAACAGGAC (SEQ ID NO: 7), AAGUGAAUUUUUACCAAACAG (SEQ ID NO: 8), UUUAUUAAGGUUUUGACUCAU (SEQ ID NO: 9), UAUACUUCCCUUCUCAAUGCC (SEQ ID NO: 10), CTTGATTAGAAGGACGGTT (SEQ ID NO: 11), AGAUCAACUGGAACCUGACAGAUUU (SEQ ID NO: 12), GCCGUGAUAUUUGGCUUCUUGGCAA (SEQ ID NO: 13), or GGCCCUGAUUGCGUUCAUCAUCUGCAUA (SEQ ID NO: 14), peptide, small molecule (including, but not limited to C29H38O4 (Celastrol), Ellipticine, C54H78N2O17 (Lobophorin F), C19H14O7 (5-Methoxysterigmatocysin), C16H12O7 (Isorhamnetin), C21H26N2O3 (Tabernaemontanin), C27H34O10 (Verrucarin A 9,10-epoxide), C10H12N4O5 (Inosine), C16H12O7, C18H23NO6, C25H33N3O·ClH, C16H12O7 (Rhamnetin), C12H13N5O4 (Toyocamycin), C17H19N3O4 (Anthramycin methyl ether), C21H22N2O3 (Paquinimod), C20H18N2O5·Na (Camptothecin sodium), C18H16O8 (Rosmarinic Acid), C10H14O3, C9H16N2O6 (Tetrahydrouridine), C10H14O3 (Mephenesin), C10H14O3 (4-Ethoxy-3-methoxybenzyl alcohol), C10H14O3 (5-tert-butyl-2-methyl-3-furoic acid), C21H20F3NO6, C23H32N2O6, C15H10N20, C36H48N2O12 (Rhoodomycin A), C16H24O5 (Ocalicine subst.), C14H25N3O9 (Kasugamycin), C4H6O2 (Crotonic Acid), C21H26N2O3 (Vincamine), Fumitremorgin C, C10H12N4O5 (Formycin B), C18H23NO6 (Swazine), C25H33N3O·ClH (Butylcycloheptylprodiginine Hydrochloride), C18H22N2O4·C6H8O7 (Quinocarcin monocitrate), C23H28N2O5, C11H13NO2·BrH, C21H24N2O3 (Strychninic acid), C21H22N2O3 (Pseudostrychnine), C20H18N2O5·Na (Camptothecin sodium), C18H16O8 (Centaureidin), C10H14O3 (Ramulosin), C19H26N2·C4H6O6, C15H10N2O6 (Lomondomycin), C21H26N2O3 (Pseudoyohimbine), or C26H28O5 (Glycyrrhizol A)), or antibody that binds to CMTM4, inhibits the activity of CMTM4, or inhibits the expression of CMTM4. For example, disclosed herein are methods of treating, inhibiting, reducing, decreasing, ameliorating, and/or preventing a cancer and/or metastasis (such as, for example, cholangiocarcinoma (CHOL), colon adenocarcinoma (COAD), esophageal carcinoma (ESCA), kidney chromophobe (KICH), pheochromocytoma and paraganglioma (PCPG), prostate adenocarcinoma (PRAD), rectum adenocarcinoma (READ), and thymoma (THYM), breast cancer, colon cancer, melanoma, prostate cancer, a glioma, kidney cancer, or lung adenocarcinoma) in a subject comprising administering to the subject an agent that inhibits CMTM4. For example, the method can comprise administering a miRNA, shRNA, siRNA (such as, for example, UUAGAUUCCAGUUGAUCUGGG (SEQ ID NO: 1), UGUUAGAUUCCAGUUGAUCUG (SEQ ID NO: 2), ACCAAAUCUGUUAGAUUCCAG (SEQ ID NO: 3), AGAAAACUUGAUUAGAAGGAC (SEQ ID NO: 4), AAGAAAACUUGAUUAGAAGGA (SEQ ID NO: 5), AGAAAGAAAACUUGAUUAGAA (SEQ ID NO: 6), UGAAUUUUUACCAAACAGGAC (SEQ ID NO: 7), AAGUGAAUUUUUACCAAACAG (SEQ ID NO: 8), UUUAUUAAGGUUUUGACUCAU (SEQ ID NO: 9), UAUACUUCCCUUCUCAAUGCC (SEQ ID NO: 10), CTTGATTAGAAGGACGGTT (SEQ ID NO: 11), AGAUCAACUGGAACCUGACAGAUUU (SEQ ID NO: 12), GCCGUGAUAUUUGGCUUCUUGGCAA (SEQ ID NO: 13), or GGCCCUGAUUGCGUUCAUCAUCUGCAUA (SEQ ID NO: 14), peptide, small molecule (including, but not limited to C29H38O4 (Celastrol), Ellipticine, C54H78N2O17 (Lobophorin F), C19H14O7 (5-Methoxysterigmatocysin), C16H12O7 (Isorhamnetin), C21H26N2O3 (Tabernaemontanin), C27H34O10 (Verrucarin A 9,10-epoxide), C10H12N4O5 (Inosine), C16H12O7, C18H23NO6, C25H33N3O·ClH, C16H12O7 (Rhamnetin), C12H13N5O4 (Toyocamycin), C17H19N3O4 (Anthramycin methyl ether), C21H22N2O3 (Paquinimod), C20H18N2O5·Na (Camptothecin sodium), C18H16O8 (Rosmarinic Acid), C10H14O3, C9H16N2O6 (Tetrahydrouridine), C10H14O3 (Mephenesin). C10H14O3 (4-Ethoxy-3-methoxybenzyl alcohol), C10H14O3 (5-tert-butyl-2-methyl-3-furoic acid), C21H20F3NO6, C23H32N2O6, C15H10N2O, C36H48N2O12 (Rhoodomycin A), C16H24O5 (Ocalicine subst.), C14H25N3O9 (Kasugamycin), C4H6O2 (Crotonic Acid), C21H26N2O3 (Vincamine), Fumitremorgin C, C10H12N4O5 (Formycin B), C18H23NO6 (Swazine), C25H33N3O·ClH (Butylcycloheptylprodiginine Hydrochloride), C18H22N2O4·C6H8O7 (Quinocarcin monocitrate), C23H28N2O5, C11H13NO2·BrH, C21H24N2O3 (Strychninic acid), C21H22N2O3 (Pseudostrychnine), C20H18N2O5·Na (Camptothecin sodium), C18H16O8 (Centaureidin), C10H14O3 (Ramulosin), C19H26N2·C4H6O6, C15H10N2O6 (Lomondomycin), C21H26N2O3 (Pseudoyohimbine), or C26H28O5 (Glycyrrhizol A)), or antibody that binds to CMTM4, inhibits the activity of CMTM4, or inhibits the expression of CMTM4.

Also disclosed herein are methods of decreasing immunosuppressive activity in a tumor microenvironment of a cancer (such as, for example, cholangiocarcinoma (CHOL), colon adenocarcinoma (COAD), esophageal carcinoma (ESCA), kidney chromophobe (KICH), pheochromocytoma and paraganglioma (PCPG), prostate adenocarcinoma (PRAD), rectum adenocarcinoma (READ), and thymoma (THYM), breast cancer, colon cancer, melanoma, prostate cancer, a glioma, kidney cancer, or lung adenocarcinoma) in a subject comprising administering to the microenvironment an agent that inhibits CMTM4. For example, the method can comprise administering a miRNA, shRNA, siRNA (such as, for example, UUAGAUUCCAGUUGAUCUGGG (SEQ ID NO: 1), UGUUAGAUUCCAGUUGAUCUG (SEQ ID NO: 2), ACCAAAUCUGUUAGAUUCCAG (SEQ ID NO: 3), AGAAAACUUGAUUAGAAGGAC (SEQ ID NO: 4), AAGAAAACUUGAUUAGAAGGA (SEQ ID NO: 5), AGAAAGAAAACUUGAUUAGAA (SEQ ID NO: 6), UGAAUUUUUACCAAACAGGAC (SEQ ID NO: 7), AAGUGAAUUUUUACCAAACAG (SEQ ID NO: 8), UUUAUUAAGGUUUUGACUCAU (SEQ ID NO: 9), UAUACUUCCCUUCUCAAUGCC (SEQ TD NO: 10), CTTGATTAGAAGGACGGTT (SEQ ID NO: 11), AGAUCAACUGGAACCUGACAGAUUU (SEQ ID NO: 12), GCCGUGAUAUUUGGCUUCUUGGCAA (SEQ ID NO: 13), or GGCCCUGAUUGCGUUCAUCAUCUGCAUA (SEQ ID NO: 14), peptide, small molecule (including, but not limited to C29H38O4 (Celastrol), Ellipticine, C54H78N2O17 (Lobophorin F), C19H14O7 (5-Methoxysterigmatocysin), C16H12O7 (Isorhamnetin), C21H26N2O3 (Tabemaemontanin), C27H34O10 (Verrucarin A 9,10-epoxide), C10H12N4O5 (Inosine), C16H12O7, C18H23NO6, C25H33N3O·ClH, C16H12O7 (Rhamnetin), C12H13N5O4 (Toyocamycin), C17H19N3O4 (Anthramycin methyl ether), C21H22N2O3 (Paquinimod), C20H18N2O5·Na (Camptothecin sodium), C18H16O8 (Rosmarinic Acid), C10H14O3, C9H16N2O6 (Tetrahydrouridine), C10H14O3 (Mephenesin), C10H14O3 (4-Ethoxy-3-methoxybenzyl alcohol), C10H14O3 (5-tert-butyl-2-methyl-3-furoic acid), C21H20F3NO6, C23H32N2O6, C15H10N2O, C36H48N2O12 (Rhoodomycin A), C16H24O5 (Ocalicine subst.), C14H25N3O9 (Kasugamycin), C4H6O2 (Crotonic Acid), C21H26N2O3 (Vincamine), Fumitremorgin C, C10H12N4O5 (Formycin B), C18H23NO6 (Swazine), C25H33N3O·ClH (Butylcycloheptylprodiginine Hydrochloride), C18H22N2O4·C6H8O7 (Quinocarcin monocitrate), C23H28N2O5, C11H13NO2·BrH, C21H24N2O3 (Strychninic acid), C21H22N2O3 (Pseudostrychnine), C20H18N2O5·Na (Camptothecin sodium), C18H16O8 (Centaureidin), C10H14O3 (Ramulosin), C19H26N2·C4H6O6, C15H10N2O6 (Lomondomycin), C21H26N2O3 (Pseudoyohimbine), or C26H28O5 (Glycyrrhizol A)), or antibody that binds to CMTM4, inhibits the activity of CMTM4, or inhibits the expression of CMTM4.

As shown herein the mere knockdown or knockout of CMTM4 in a cancer can still results in metastasis in some cancer as neutrophils can modulate M1 macrophages functional phenotype toward to have M2-dependent phenotypes especially in the metabolic pathways and oxidative stress in an inflammatory condition that promotes tumor development and progression. Thus, in some aspects, the method of treating, inhibiting, reducing, decreasing, ameliorating, and/or preventing a cancer and/or metastasis further comprise the administration of anti-inflammatory agents (such as, for example, an agent that inhibits LPS, IL-1β, IFNγ, TNF-α, and/or S100A8) and/or antibodies that bind to neutrophils (such as, for example, anti-Ly6G neutralizing antibodies).

In one aspect, the method of treatment can further comprise the administration of an epidermal growth factor receptor (EGFR) inhibitor (such as, for example, erlotinib, osimertinib, neratinib, gefitinib, cetuximab, pantibumumab, dacomitinib, lapatinib, necitumumab, mobocertinib, and vandetanib) or a platelet-derived growth factor receptor A (PDGFRa) inhibitor (such as, for example, avapritinib, imatinib, and ripretinib).

It is understood and herein contemplated that the disclosed treatment regimens can used alone or in combination with any anti-cancer therapy known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine 1131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). The treatment methods can include or further include checkpoint inhibitors including, but are not limited to antibodies that block PD-1 (Nivolumab (BMS-936558 or MDX1106), CT-011, MK-3475), PD-L1 (MDX-1105 (BMS-936559), MPDL3280A, or MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016).

It is understood and herein contemplated that expression of IL-6 in the tumor microenivornment or the association of CMTM4 with Rab35 can have an immunosuppressive effect with IL-6 being an inhibitory cytokine and Rab35/CMTM4 association having an effect on protein recycling. As disclosed herein, CMTM4 deletion results in reduced association of RAB 35 with CMTM4, thereby reducing protein recycle and favoring the lysosome pathway. As tumor antigen and MHC Class II are assembled in the lysosome followed by presentation of the antigen to the cell surface., the disruption of the Rab35/CMTM4 association thus leads to higher MHC Class II assembling and surface expression upon IFNγ stimulation. Accordingly, disclosed herein are methods of decreasing IL-6 expression and/or activity and/or increasing MHCII expression in a tumor microenvironment of a cancer (such as, for example, cholangiocarcinoma (CHOL), colon adenocarcinoma (COAD), esophageal carcinoma (ESCA), kidney chromophobe (KICH), pheochromocytoma and paraganglioma (PCPG), prostate adenocarcinoma (PRAD), rectum adenocarcinoma (READ), and thymoma (THYM), breast cancer, colon cancer, melanoma, prostate cancer, a glioma, kidney cancer, or lung adenocarcinoma) in a subject comprising administering to the microenvironment an agent that inhibits CMTM4. For example, the method can comprise administering a miRNA, shRNA, siRNA (such as, for example, UUAGAUUCCAGUUGAUCUGGG (SEQ ID NO: 1), UGUUAGAUUCCAGUUGAUCUG (SEQ ID NO: 2), ACCAAAUCUGUUAGAUUCCAG (SEQ ID NO: 3), AGAAAACUUGAUUAGAAGGAC (SEQ ID NO: 4), AAGAAAACUUGAUUAGAAGGA (SEQ ID NO: 5), AGAAAGAAAACUUGAUUAGAA (SEQ ID NO: 6), UGAAUUUUUACCAAACAGGAC (SEQ ID NO: 7), AAGUGAAUUUUUACCAAACAG (SEQ ID NO: 8), UUUAUUAAGGUUUUGACUCAU (SEQ ID NO: 9), UAUACUUCCCUUCUCAAUGCC (SEQ ID NO: 10), CTTGATTAGAAGGACGGTT (SEQ ID NO: 11), AGAUCAACUGGAACCUGACAGAUUU (SEQ ID NO: 12), GCCGUGAUAUUUGGCUUCUUGGCAA (SEQ ID NO: 13), or GGCCCUGAUUGCGUUCAUCAUCUGCAUA (SEQ ID NO: 14), peptide, small molecule (including, but not limited to C29H38O4 (Celastrol), Ellipticine, C54H78N2O17 (Lobophorin F), C19H14O7 (5-Methoxysterigmatocysin), C16H12O7 (Isorhamnetin), C21H26N2O3 (Tabernaemontanin), C27H34O10 (Verrucarin A 9,10-epoxide), C10H12N4O5 (Inosine), C16H12O7, C18H23NO6, C25H33N3O·ClH, C16H12O7 (Rhamnetin), C12H13N5O4 (Toyocamycin), C17H19N3O4 (Anthramycin methyl ether), C21H22N2O3 (Paquinimod), C20H18N2O5·Na (Camptothecin sodium), C18H16O8 (Rosmarinic Acid), C10H14O3, C9H16N2O6 (Tetrahydrouridine), C10H14O3 (Mephenesin), C10H14O3 (4-Ethoxy-3-methoxybenzyl alcohol), C10H14O3 (5-tert-butyl-2-methyl-3-furoic acid), C21H20F3NO6, C23H32N2O6, C15H10N2O, C36H48N2O12 (Rhoodomycin A), C16H24O5 (Ocalicine subst.), C14H25N3O9 (Kasugamycin), C4H6O2 (Crotonic Acid), C21H26N2O3 (Vincamine), Fumitremorgin C, C10H12N4O5 (Formycin B), C18H23NO6 (Swazine), C25H33N3O·ClH (Butylcycloheptylprodiginine Hydrochloride), C18H22N2O4·C6H8O7 (Quinocarcin monocitrate), C23H28N2O5, C11H13NO2·BrH, C21H24N2O3 (Strychninic acid), C21H22N2O3 (Pseudostrychnine), C20H18N2O5·Na (Camptothecin sodium), C18H16O8 (Centaureidin), C10H14O3 (Ramulosin), C19H26N2·C4H6O6, C15H10N2O6 (Lomondomycin), C21H26N2O3 (Pseudoyohimbine), or C26H28O5 (Glycyrrhizol A)), or antibody that binds to CMTM4, inhibits the activity of CMTM4, or inhibits the expression of CMTM4.

### 1. Homology/Identity

It is understood that one way to define any known variants and derivatives or those that might arise, of the disclosed genes and proteins herein is through defining the variants and derivatives in terms of homology to specific known sequences. For example, SEQ ID Nos: 1-14 set forth a particular sequence of CMTM4 inhibitory siRNA. Specifically disclosed are variants of these and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

### 2. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

### 3. Nucleic Acids

There are a variety of molecules disclosed herein that are nucleic acid based, including for example the nucleic acids that comprise, for example SEQ ID Nos: 1-14, or fragments thereof, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

### A) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate). There are many varieties of these types of molecules available in the art and available herein.

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties. There are many varieties of these types of molecules available in the art and available herein.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. There are many varieties of these types of molecules available in the art and available herein.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556). There are many varieties of these types of molecules available in the art and available herein.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

### b) Functional Nucleic Acids

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of any of the disclosed nucleic acids, such as CMTM4. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d}) less than or equal to 10⁻⁶, 10⁻⁸, 10⁻¹⁰, or 10⁻¹². A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

### 4. Delivery of the Compositions to Cells

There are a number of compositions and methods which can be used to deliver nucleic acids to cells, either in vitro or in vivo. These methods and compositions can largely be broken down into two classes, viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

### A) Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as any one or more of SEQ ID Nos: 1-14 into the cell without degradation and include a promoter yielding expression of the gene in the cells into which it is delivered. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

### (1) Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

### (2) Adenoviral Vectors

The construction of replication-defective adenoviruses has been described (Berkner et al., J Virology 61:1213-1220 (1987); Massie et al., Mol. Cell. Biol. 6:2872-2883 (1986); Haj-Ahmad et al., J Virology 57:267-274 (1986); Davidson et al., J Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

### (3) Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, CA, which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

### (4) Large Payload Viral Vectors

Molecular genetic experiments with large human herpesviruses have provided a means whereby large heterologous DNA fragments can be cloned, propagated and established in cells permissive for infection with herpesviruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA >150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA >220 kb and to infect cells that can stably maintain DNA as episomes.

Other useful systems include, for example, replicating and host-restricted non-replicating vaccinia virus vectors.

### B) Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed CMTM4 inhibitory agents including, but not limited to miRNA, shRNA, siRNA (such as, for example, UUAGAUUCCAGUUGAUCUGGG (SEQ ID NO: 1), UGUUAGAUUCCAGUUGAUCUG (SEQ ID NO: 2), ACCAAAUCUGUUAGAUUCCAG (SEQ ID NO: 3), AGAAAACUUGAUUAGAAGGAC (SEQ ID NO: 4), AAGAAAACUUGAUUAGAAGGA (SEQ ID NO: 5), AGAAAGAAAACUUGAUUAGAA (SEQ ID NO: 6), UGAAUUUUUACCAAACAGGAC (SEQ ID NO: 7), AAGUGAAUUUUUACCAAACAG (SEQ ID NO: 8), UUUAUUAAGGUUUUGACUCAU (SEQ ID NO: 9), UAUACUUCCCUUCUCAAUGCC (SEQ ID NO: 10), CTTGATTAGAAGGACGGTT (SEQ ID NO: 11), AGAUCAACUGGAACCUGACAGAUUU (SEQ ID NO: 12), GCCGUGAUAUUUGGCUUCUUGGCAA (SEQ ID NO: 13), or GGCCCUGAUUGCGUUCAUCAUCUGCAUA (SEQ ID NO: 14), peptide, small molecule (including, but not limited to C29H38O4 (Celastrol), Ellipticine, C54H78N2O17 (Lobophorin F), C19H14O7 (5-Methoxysterigmatocysin), C16H12O7 (Isorhamnetin), C21H26N2O3 (Tabernaemontanin), C27H34O10 (Verrucarin A 9,10-epoxide), C10H12N4O5 (Inosine), C16H12O7, C18H23NO6, C25H33N3O·ClH, C16H12O7 (Rhamnetin), C12H13N5O4 (Toyocamycin), C17H19N3O4 (Anthramycin methyl ether), C21H22N2O3 (Paquinimod), C20H18N2O5·Na (Camptothecin sodium), C18H16O8 (Rosmarinic Acid), C10H14O3, C9H16N2O6 (Tetrahydrouridine), C10H14O3 (Mephenesin), C10H14O3 (4-Ethoxy-3-methoxybenzyl alcohol), C10H14O3 (5-tert-butyl-2-methyl-3-furoic acid), C21H20F3NO6, C23H32N2O6, C15H10N2O, C36H48N2O12 (Rhoodomycin A), C16H24O5 (Ocalicine subst.), C14H25N3O9 (Kasugamycin), C4H6O2 (Crotonic Acid), C21H26N2O3 (Vincamine), Fumitremorgin C, C10H12N4O5 (Formycin B), C18H23NO6 (Swazine), C25H33N3O·ClH (Butylcycloheptylprodiginine Hydrochloride), C18H22N2O4·C6H8O7 (Quinocarcin monocitrate), C23H28N2O5, C11H13NO2·BrH, C21H24N2O3 (Strychninic acid), C21H22N2O3 (Pseudostrychnine), C20H18N2O5·Na (Camptothecin sodium), C18H16O8 (Centaureidin), C10H14O3 (Ramulosin), C19H26N2·C4H6O6, C15H10N2O6 (Lomondomycin), C21H26N2O3 (Pseudoyohimbine), or C26H28O5 (Glycyrrhizol A)), or antibody that binds to CMTM4, inhibits the activity of CMTM4, or inhibits the expression of CMTM4, vectors, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, MD), SUPERFECT (Qiagen, Inc. Hilden, Germany) and TRANSFECTAM (Promega Biotec, Inc., Madison, WI), as well as other liposomes developed according to procedures standard in the art. In addition, the disclosed nucleic acid or vector can be delivered in vivo by electroporation, the technology for which is available from Genetronics, Inc. (San Diego, CA) as well as by means of a SONOPORATION machine (ImaRx Pharmaceutical Corp., Tucson, AZ).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

### C) In Vivo/Ex Vivo

As described above, the compositions can be administered in a pharmaceutically acceptable carrier and can be delivered to the subject=s cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

### 5. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### D. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: CMTM4 Regulates Inflammatory Responses Through Modulating Receptor Tyrosine Kinases to Establish a Suppressive Tumor Microenvironment for Tumor Invasion

### a) Results

### (1) CMTM4 is Highly Expressed in Cancers and can be a Prognostic Marker

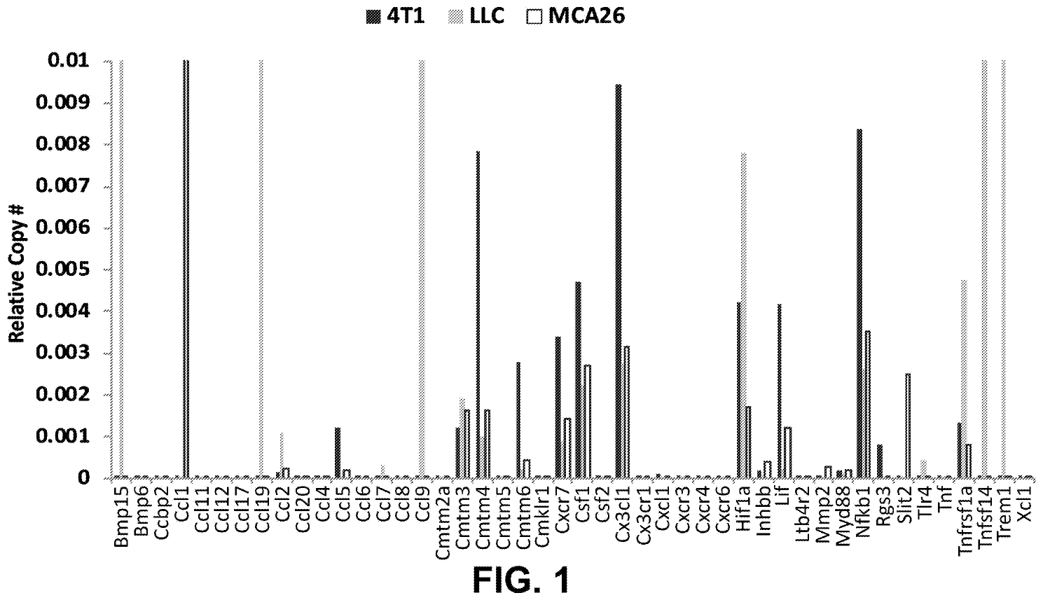

To identify a master regulator of cancer-related inflammation, we performed real-time PCR super arrays to profile gene expression in different murine tumor types. While we found several genes that have been implicated in tumor inflammation (FIG. 1), CMTM members stood out since their role in the regulation of inflammation is unclear. Since CMTM4 is highly expressed in various tumors examined and correlates with disease progression, we focused our study on CMTM4-mediated regulation of cancer-related inflammation and subsequent changes to the host tumor immunity for tumor invasion.

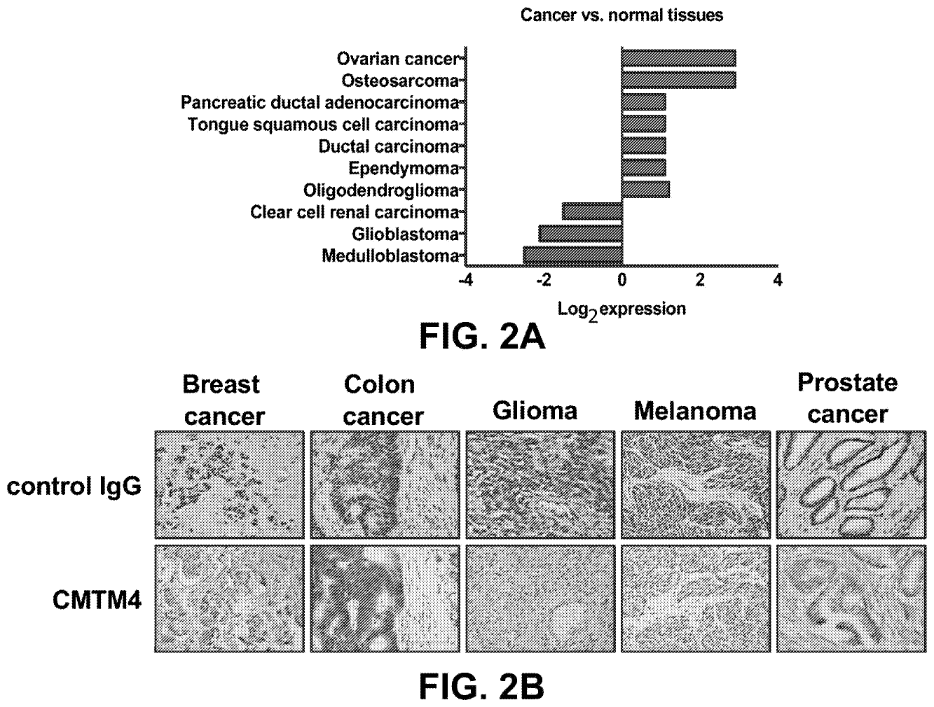

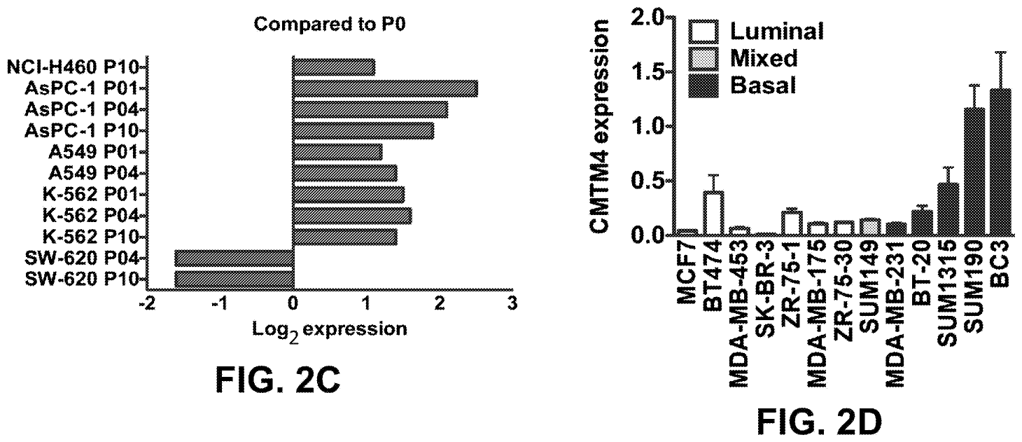

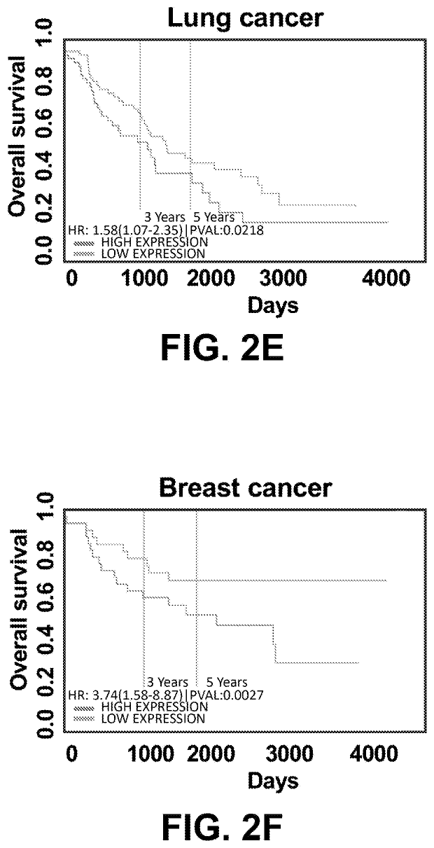

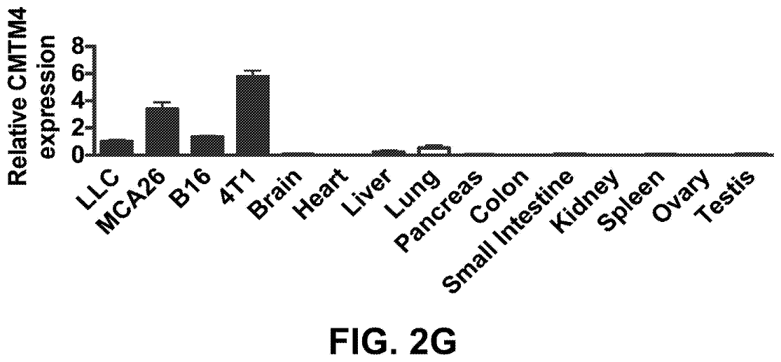

To determine whether CMTM4 expression is increased in human carcinomas, CMTM4 expression was compared in multiple human cancer types from TCGA and Genotype Tissue Expression (GTEx) databases. We found that CMTM4 expression was significantly higher in cholangiocarcinoma (CHOL), colon adenocarcinoma (COAD), esophageal carcinoma (ESCA), kidney chromophobe (KICH), pheochromocytoma and paraganglioma (PCPG), prostate adenocarcinoma (PRAD), rectum adenocarcinoma (READ), and thymoma (THYM) as compared to normal tissues (FIG. 2A). We also confirmed CMTM4 expressions in a variety of human carcinoma tissue biopsies. Tissue sections from breast cancer, colon cancer, and prostate cancer patients showed high levels of CMTM4 expression. Glioma and melanoma tissues also showed CMTM4 expression (FIG. 2B). Furthermore, CMTM4 expression in the advanced lung adenocarcinoma indicated that stage IV showed much higher expression of CMTM4 compared to the lower stage of lung adenocarcinoma, indicating a correlation between CMTM4 expression and aggressiveness of tumors. (FIG. 2C). To determine whether the degree of CMTM4 expression is correlated with the breast cancer subtype, its expression levels in breast cancer cell lines classified as luminal, mixed, and invasive basal types were compared. Interestingly, invasive basal breast cancer cell lines exhibited higher CMTM4 expression levels compared to luminal and mixed breast cancer cell lines, indicating an association between CMTM4 expression level with tumor progression and poor outcome (FIG. 2D). We next investigated the correlation between CMTM4 expression and cancer patient survival. Kaplan-Meier analysis indicated that lung and breast cancer patient groups with higher CMTM4 expression showed a significantly reduced survival rate in comparison to the group with lower CMTM4 expression (FIGS. 2E and 2F). Adrenal, brain, head, and neck cancer, and leukemia patients also showed lower survival in the patient group that had higher CMTM4 expression (Table 1). A series of quantitative real-time PCR assays confirmed that the expression of CMTM4 was significantly higher in tumor cell lines than in the normal tissues analyzed (FIG. 2G). Overall, these data indicate that CMTM4 has a strong expression in multiple cancer types, correlates with tumor progression, and can be a poor prognostic factor in multiple types of cancer patients.

TABLE 1
Overall survival rates in carcinoma patients
were correlated with CMTM4 expression level.
CMTM4

| Cancer type | p value | data source |
|---|---|---|
| Adrenal | 0.0778 | GSE33371 |
| | 0.0229 | GSE19776 |
| Brain | 0.05 | GSE7696 |
| | 0.003 | GSE4271 |
| Breast | 0.0027 | GSE37751 |
| (ER neg) | 0.003 | GSE37751 |
| (PR neg) | 0.02 | TCGA |
| (Chemo pos) | 0.001 | GSE37751 |
| Colon | 0.02 | TCGA |
| Myeloid Leukemia | 0.0277 | TCGA |
| HNC | 0.049 | TCGA |
| Lung | 0.02 | GSE26939 |
| (Grade 2) | 0.035 | GSE26939 |
| Renal | 0.003 | TCGA |
| Neuro-endocrine cancer | 0.0002272 | GSE62564 |

### (2) CMTM4 is Involved in Tumor Progression In Vivo

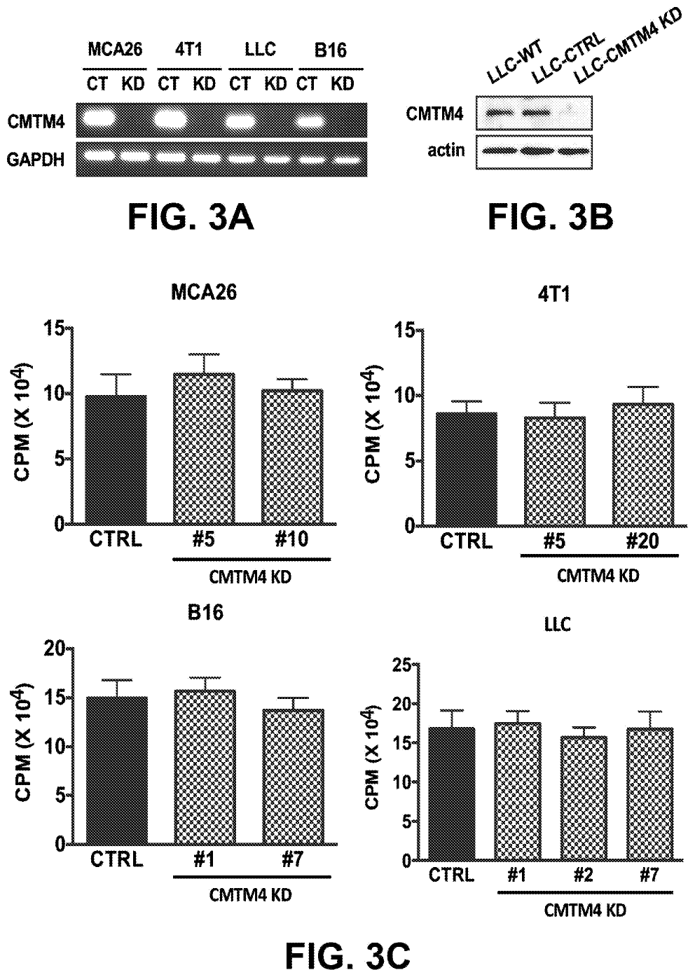

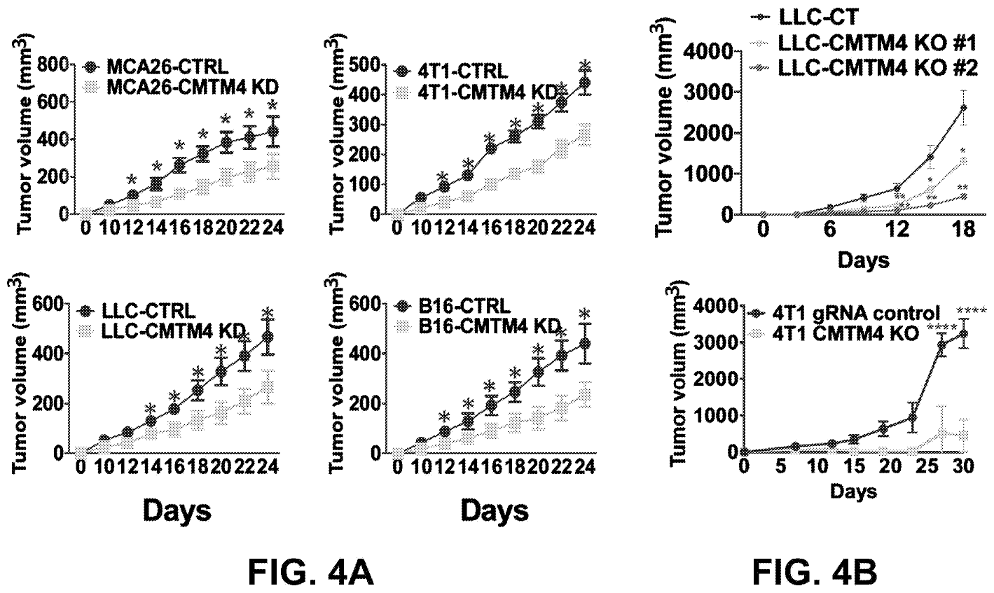

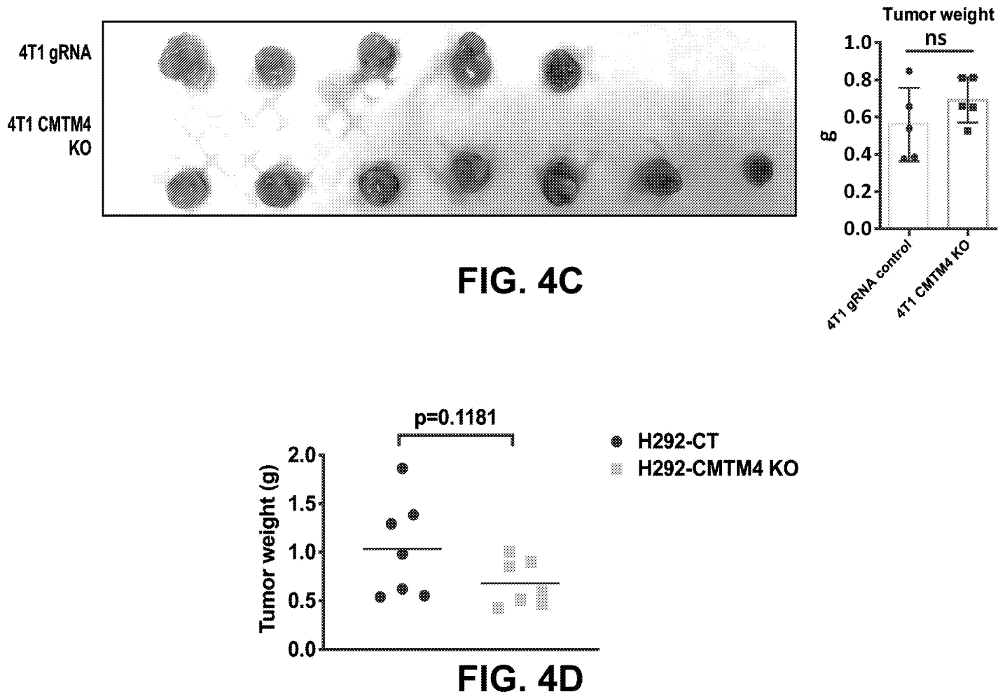

To investigate the function of CMTM4 in tumors, four different murine tumor cell lines were transfected with siRNA vectors targeting murine CMTM4 mRNA, or a control vector. Successful knockdown (KD) was confirmed by RT-PCR (FIG. 3A) and western blot (FIG. 3B). We evaluated the effect of CMTM4 KD on in vitro proliferation of tumor cells. CMTM4 KD exerted no significant effect on the proliferation of the four tumor cell lines analyzed (FIG. 3C). Since in vitro assays cannot recapitulate the effect of host cells and other factors present in vivo, we also evaluated the effect of CMTM4 KD on tumor progression in mice. Interestingly, CMTM4 KD clones of the four tumor cell lines grew significantly slower than their respective control vector-transfected clones (FIGS. 4A and 4B). CMTM4 KO generated by the CRISPR KO system showed a significant decrease in tumor growth compared to control in LLC and 4T1 tumor models (FIGS. 4A and 4B). However, CMTM4 KO did not suppress in vivo tumor growth in immunodeficient mice (FIGS. 4C and 4D). These results indicate that CMTM4 does not affect the intrinsic growth rate of the tumor cells but may play an important role in mediating the interaction between tumor cells and the surrounding tumor microenvironment in the host.

### (3) CMTM4 Expressed on Tumor Affects Infiltration and Function of Tumor-Infiltrating Leukocytes

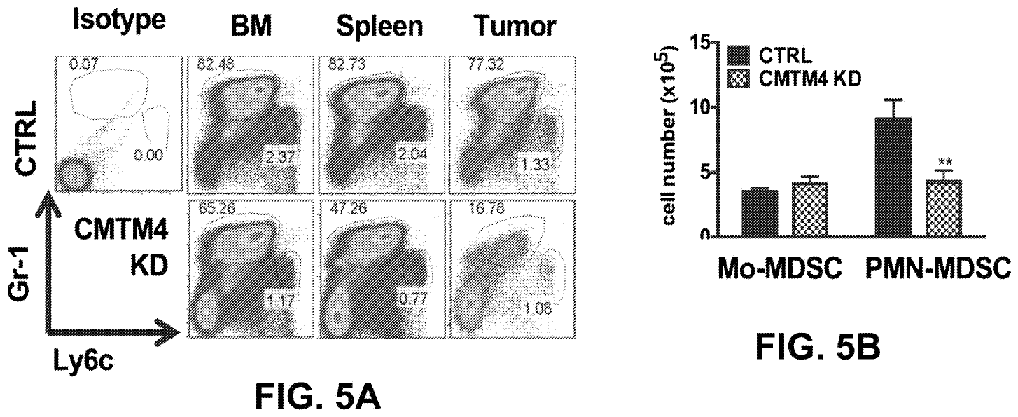

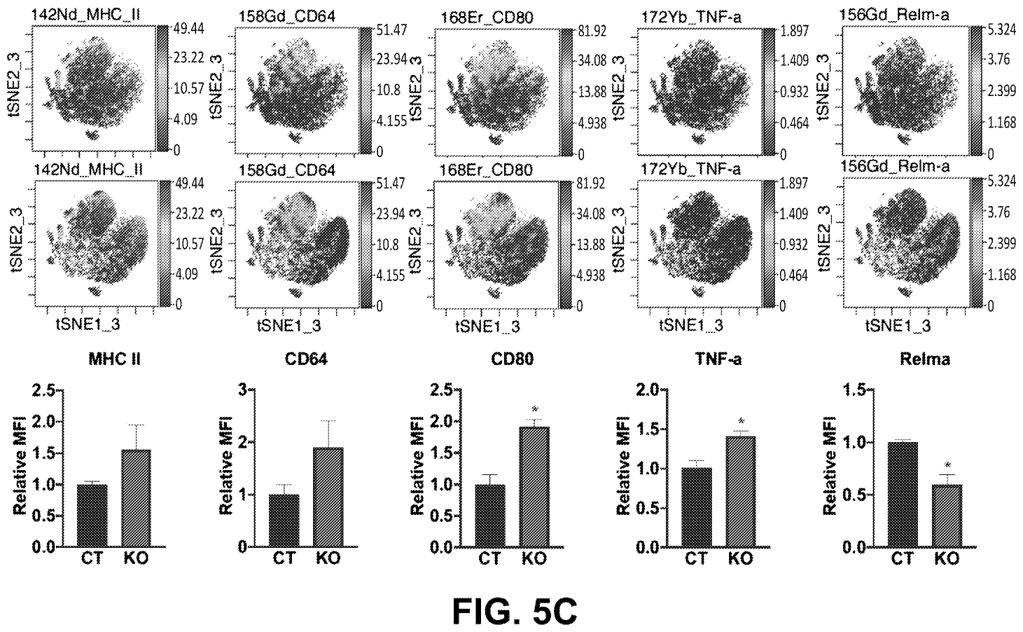

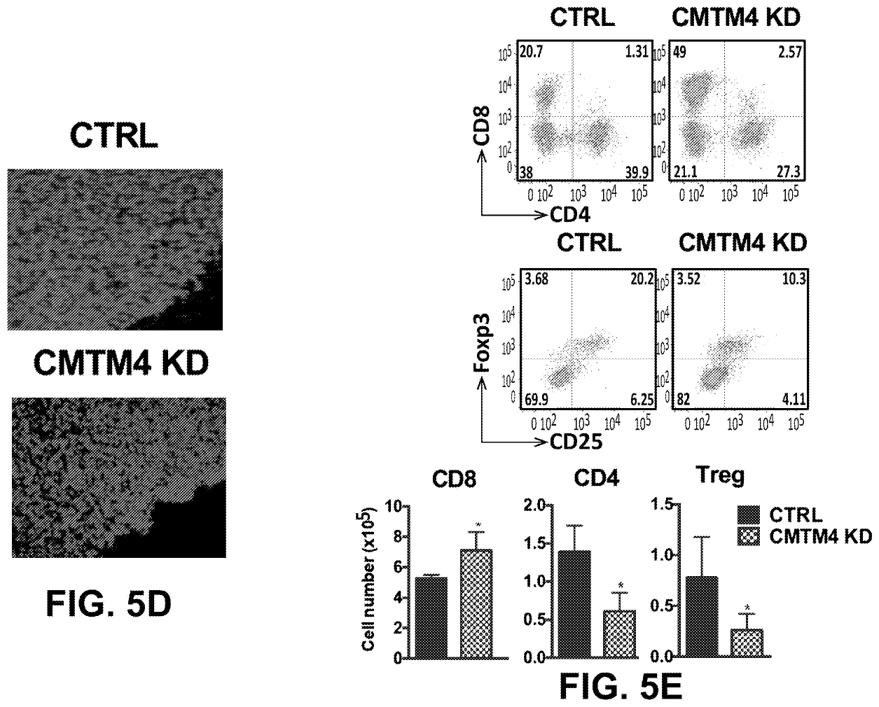

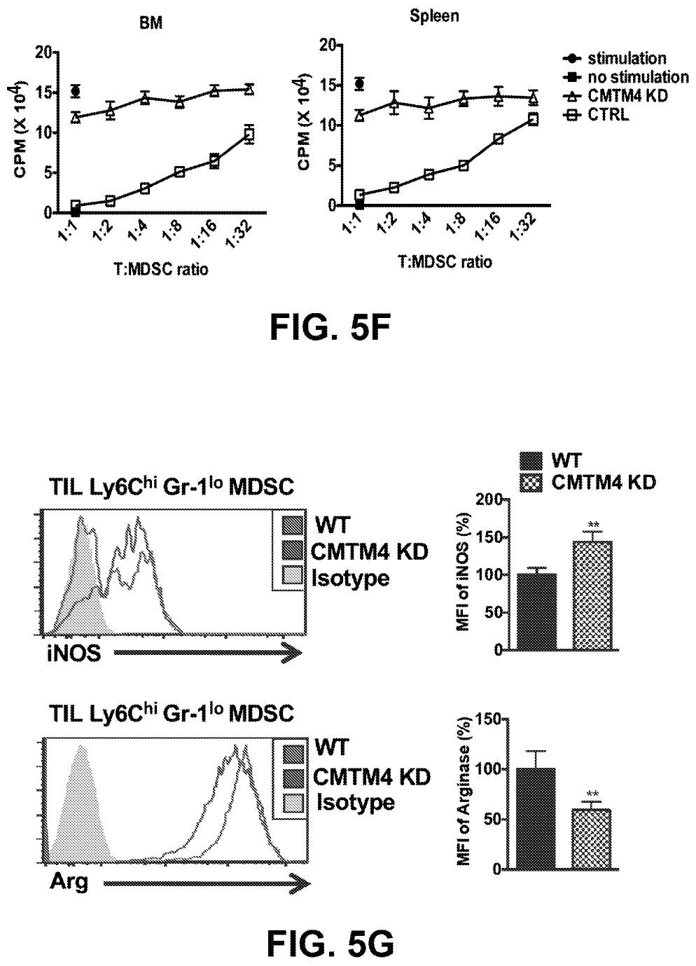

We next evaluated the effect of CMTM4 on leukocyte subset composition in the tumor. We first focused on the subsets of myeloid cells including MDSCs that have been shown to accumulate in hosts with advanced malignancies, suppress the antitumor immune response, and promote tumor angiogenesis. We found that increased infiltration of MHC II, CD64, CD80, and TNF-α expressing myeloid cells whereas reduced RELMα-expressing myeloid cells in LLC CMTM4 KD tumor-infiltrated leukocytes (FIGS. 2A and 2B). We further found significant reductions in the number of PMN (Gr-1^{High}Ly6C^{Low}) MDSCs in the bone marrow, spleen, and tumors of CMTM4 KD tumor-bearing mice compared to mice bearing control tumors. Interestingly, no substantial decrease in monocytic (Gr-1^{Low}Ly6C^{High}) MDSCs was observed within LLC CMTM4 KD tumor tissue. Fewer PMN-MDSCs within the spleen and bone marrow in mice bearing CMTM4 KD LLC tumors most likely represent an effect of impaired PMN-MDSC accumulation. We further found that CyTOF analysis revealed increased M1-like phenotypes showing increased expression of MHC II, CD64, CD80, and TNF-α but reduced M2 markers, Relma, in myeloid cells from CMTM4 KD LLC tumors (FIG. 5C). Thus, these data indicate that CMTM4 KD in LLC tumor cells results in reduced PMN-MDSC and elevated M1-like myeloid cell accumulation in the tumor.

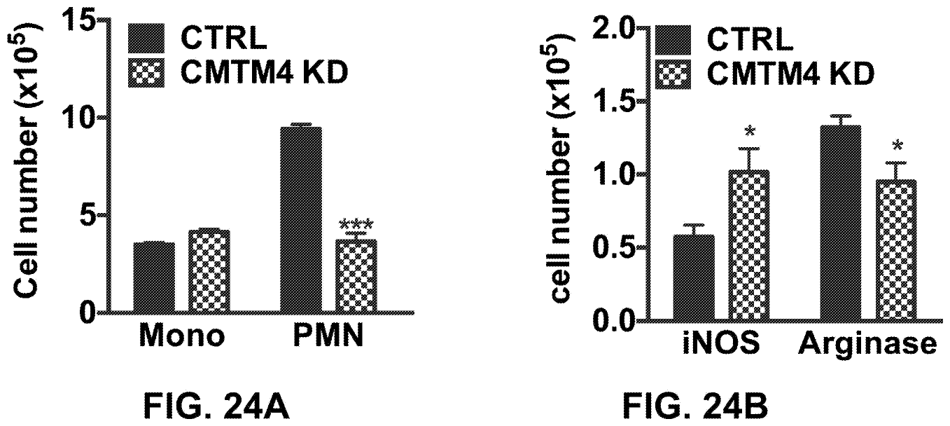

We further determined whether CMTM4 plays a role in regulating the suppressive capacity of monocytic MDSCs. Monocytic MDSCs from LLC CMTM4 KD tumor-bearing mice showed significantly diminished suppressive activities towards OT-II T-cell proliferation (FIG. 5F). Interestingly, MDSCs from CMTM4 KD tumors showed significantly higher levels of iNOS expression, an M1 phenotypic marker, and simultaneously decreased expression of arginase 1, an M2 phenotypic marker (FIG. 5G). The results indicate that the CMTM4 expressed by tumor tissues can modulate the function of tumor-infiltrating monocytic MDSCs. While decreased numbers of PMN-MDSCs were observed within CMTM4 KD tumors, there was also a reduction in the number of PMN-MDSCs in bone marrow and spleen (FIG. 5A). Therefore, we evaluated whether the reduction of MDSCs within tumor tissue could be attributed to an overall decrease in MDSC accumulation, alone or in combination with reduced recruitment, to the tumor. Sorted MDSCs from CD45.1 tumor-bearing mice were adoptively transferred into control or CMTM4 KD LLC tumor-bearing MaFIA (macrophage Fas-induced apoptosis) mice that had been depleted of CD115′ cells. Three days after adoptive transfer, significantly lower numbers of PMN-MDSCs were present in CMTM4 KD tumors whereas no significant differences were observed in the number of tumor-infiltrating monocytic MDSCs (FIG. 24A). Interestingly, iNOS' MDSC infiltration was increased and Arg1⁺ MDSC infiltration was reduced in the CMTM4 KD tumors (FIG. 24B). These results indicate that the CMTM4 expressed within tumor cells regulates tumor-infiltration of PMN-MDSCs.

### (4) CMTM4 Controls the Expression and Activation of Receptor Tyrosine Kinases

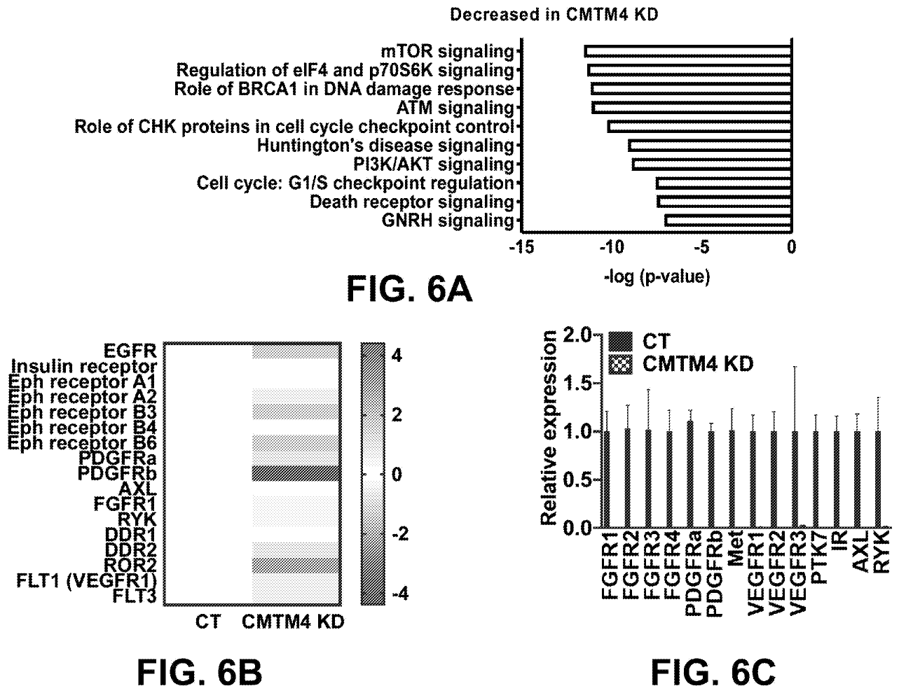

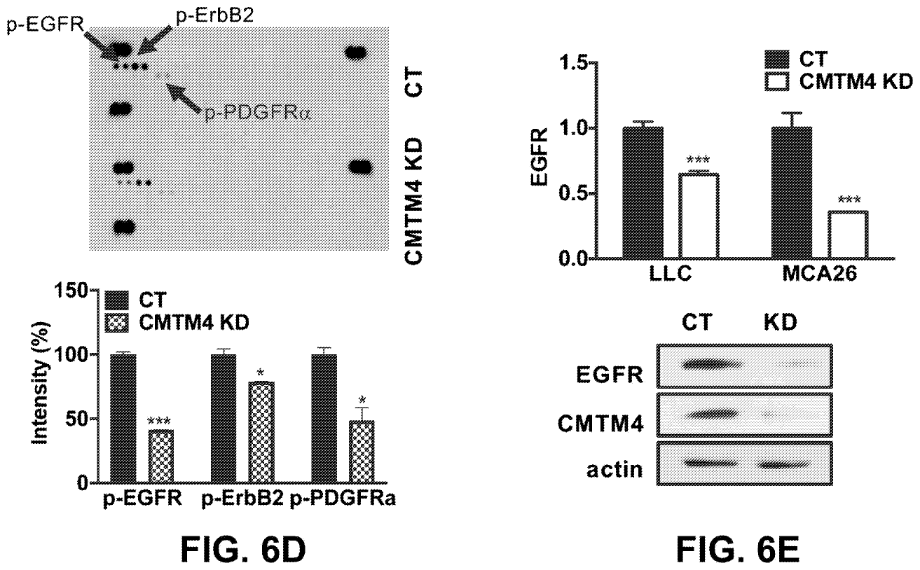

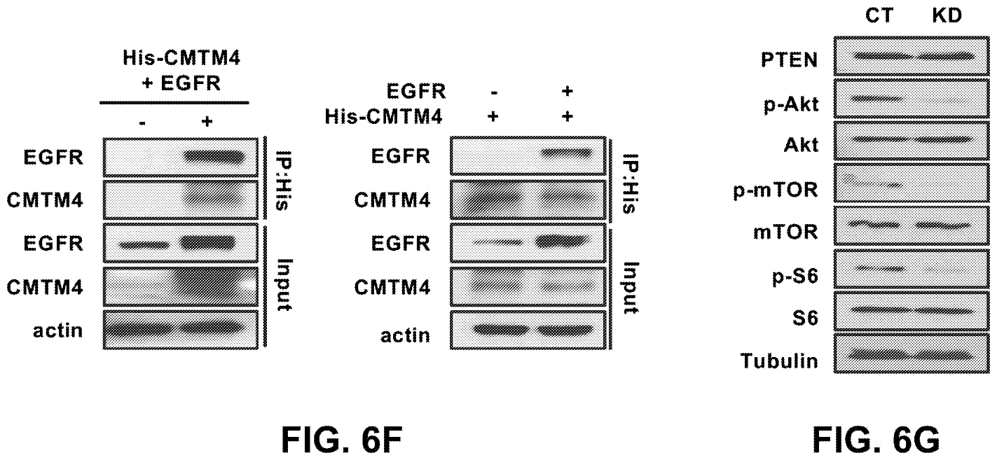

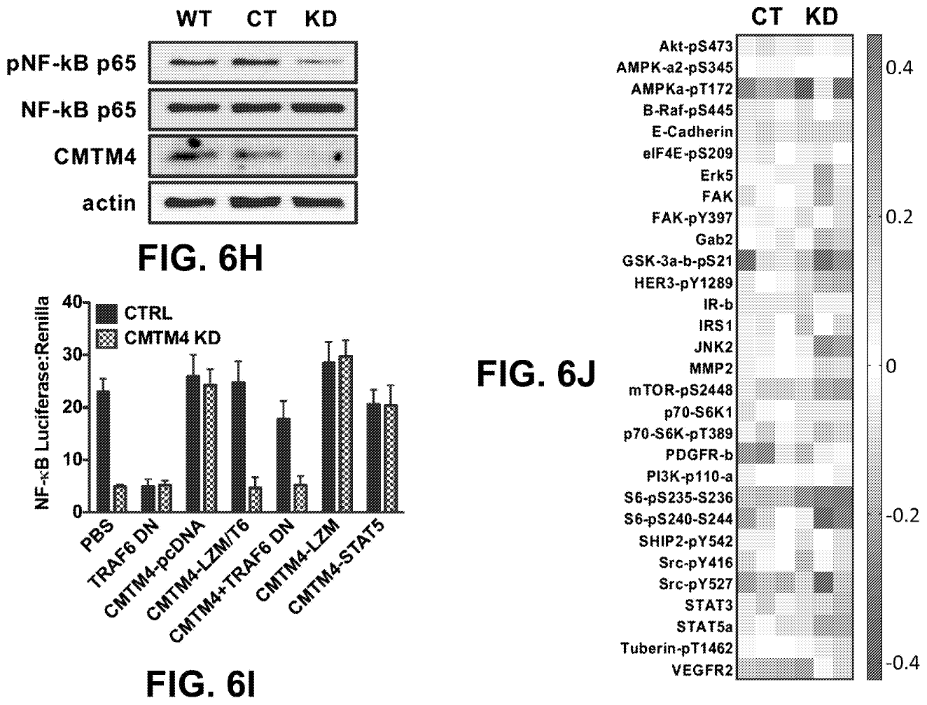

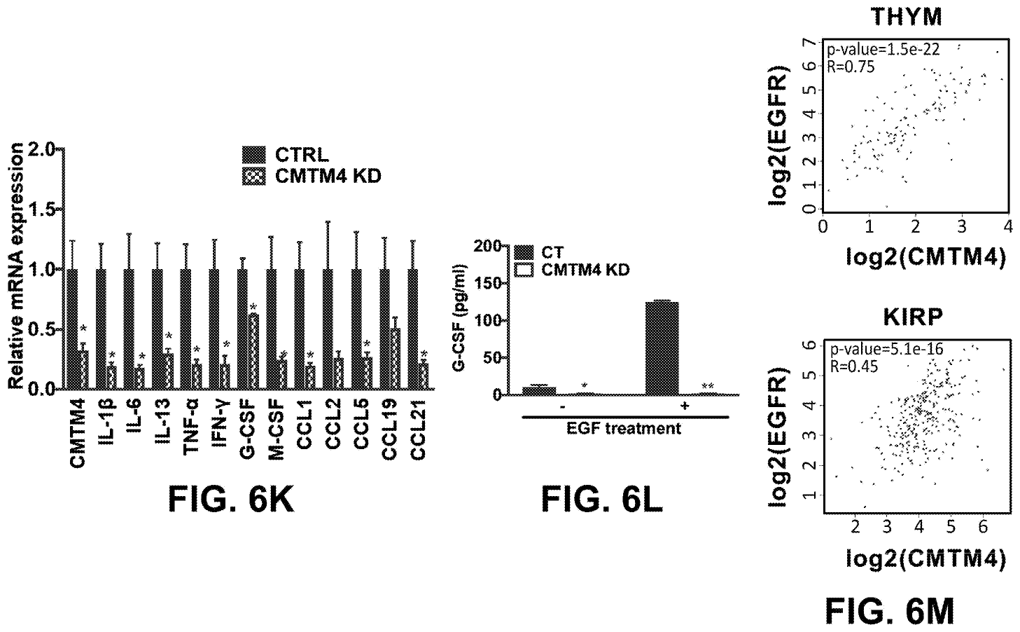

To gain insights into mechanisms by which CMTM4 regulates tumor progression, we performed RNAseq with Illumina Platform PE15 on LLC CT and CMTM4 KD cells. Ingenuity pathway analysis (IPA) of RNAseq data indicated the most significant decrease in mTOR signaling and PI3K/Akt signaling in CMTM4 KD cells compared to control (FIG. 6A). Since PI3K/Akt and mTOR are activated by receptor tyrosine kinases (RTKs), we compared RTK expression in control and CMTM4 KD cells and RNAseq analysis revealed downregulated expression of RTKs in CMTM4 KD cells compared to control cells (FIG. 6B). Decreased multiple RTKs' expression in CMTM4 KD cells was confirmed in mRNA levels (FIG. 6C). Phosphorylated RTK array indicated that overall RTK phosphorylation was reduced by CMTM4 KD. Especially, phosphorylation of epidermal growth factor receptor (EGFR), ErbB2, and PDGFRa was significantly downregulated in CMTM4 KD cells (FIG. 6D).

### (5) CMTM4 Regulates Tumor Inflammation Through Controlling Activation of EGFR Signaling

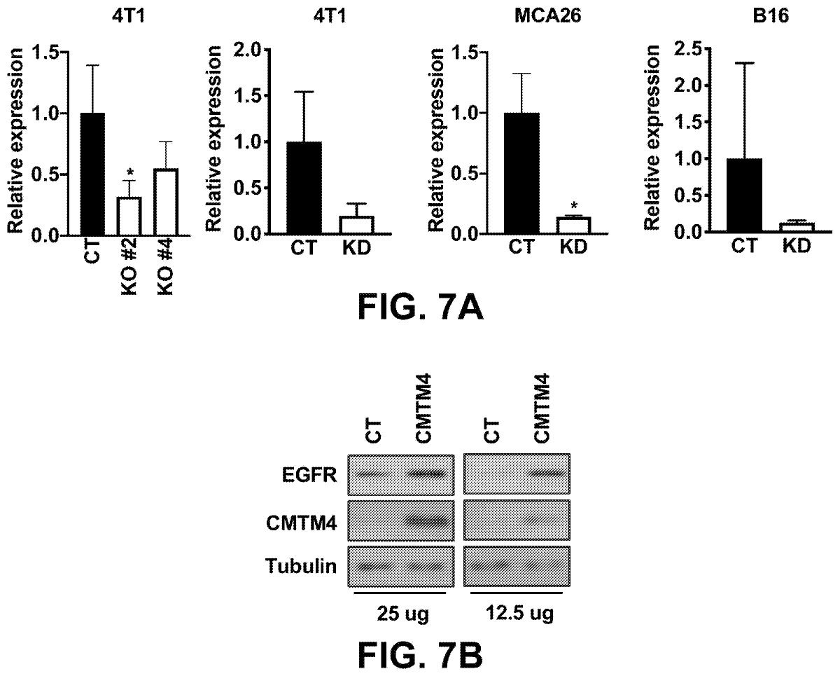

We further used EGFR as an example to evaluate the relationship between CMTM4 and RTKs. We found that CMTM4 KD/KO reduced EGFR mRNA in multiple tumor cells and protein in LLC cells (FIG. 6E, FIG. 7A). Interestingly, overexpression of CMTM4 also increased EGFR expression (FIG. 6F). The co-IP analysis revealed that CMTM4 was associated with EGFR (FIG. 7B). Next, we evaluated whether EGFR downstream signaling was influenced by CMTM4 KD. We verified that phosphorylation of Akt, mTOR, and S6 was reduced in CMTM4 KD cells compared to control cells (FIG. 6G). The expression of PTEN, which is a natural inhibitor of PI3K/Akt, was not affected by CMTM4 KD (FIG. 6G) indicating that reduced activation of Akt/mTOR signaling is not due to PTEN regulation by CMTM4. Furthermore, another downstream of Akt, NF-κB phosphorylation was inhibited by CMTM4 KD in LLC cells (FIG. 6H) and the promoter activity of NF-κB was also attenuated after CMTM4 KD, as assessed by NF-κB luciferase assay (FIG. 6I). The inhibitory effect of CMTM4 KD on NF-κB activity was restored by transfection with CMTM4 (FIG. 6I). To determine which domain of CMTM4 was critical for exerting the observed effects on NF-κB activation, multiple truncated clones of CMTM4 were generated in which one or more of its predicted binding domains had been deleted. NF-κB transcriptional activity was restored in LLC CMTM4 KD cells upon transfection with full-length CMTM4, STAT5 domain or leucine-zipper deleted CMTM constructs, but not the TRAF6 deleted CMTM4 construct (FIG. 6I). Furthermore, transfection with dominant-negative TRAF6 completely inhibited NF-κB transcriptional activation in control (CMTM4 wild-type) tumor cells (FIG. 6I). RPPA data analysis also confirmed suppressed RTK downstream expression by CMTM4 KD (FIG. 6J). Since it has been reported that mTOR signaling in cancer cells recruits MDSCs through regulating G-CSF, receptor tyrosine kinase activates PI3K/AKT and mTOR signaling, and NF-κB is a well-known regulator of inflammatory mediators, we analyzed inflammation-related cytokines and chemokines in control vs. CMTM4 KD tumor cells. Interestingly, CMTM4 KD in LLC cells resulted in a significant downregulation of cytokines and chemokines (FIG. 6K). We confirmed whether the production of G-CSF, which has been shown to promote granulopoiesis and PMN-MDSC accumulation, was influenced by CMTM4-mediated EGFR signaling. EGF treatment promoted the production of G-CSF in LLC control cells whereas G-CSF was not detectable in CMTM4 KD cells regardless of EGF stimulation (FIG. 6L). Collectively, the data demonstrate that CMTM4 is associated with and regulates RTKs and is an important regulator of EGFR/Akt signaling to drive inflammatory mediators e.g. G-CSF and cytokines that promote the accumulation of immunosuppressive MDSC in the tumor microenvironment.

### (6) CMTM4 Regulates the Re-Organization of Lipid Raft Following EGFR Release

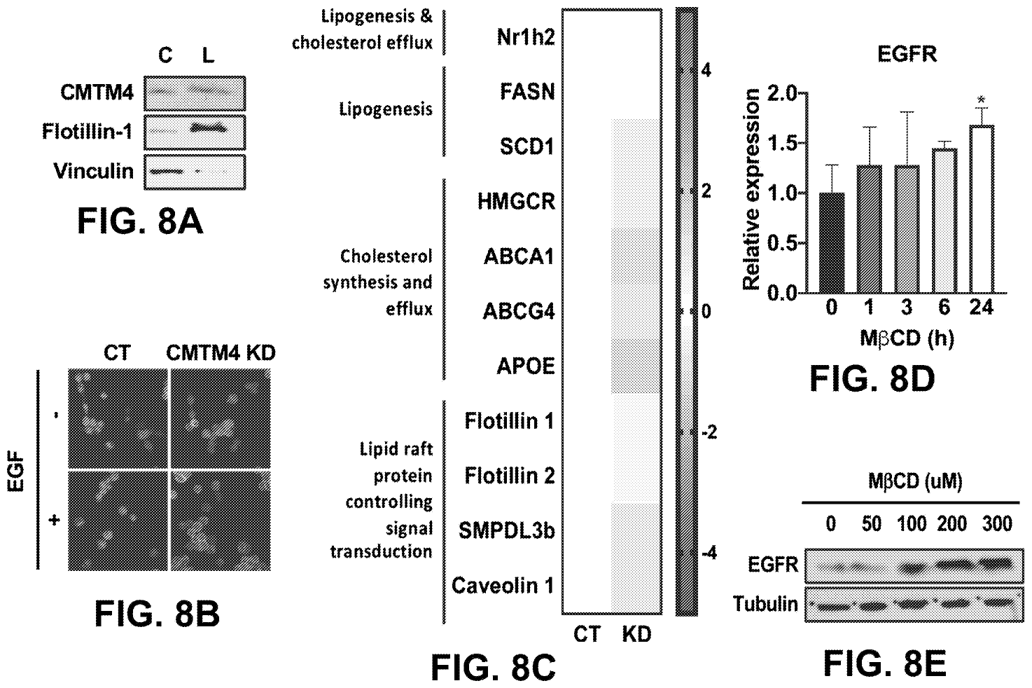

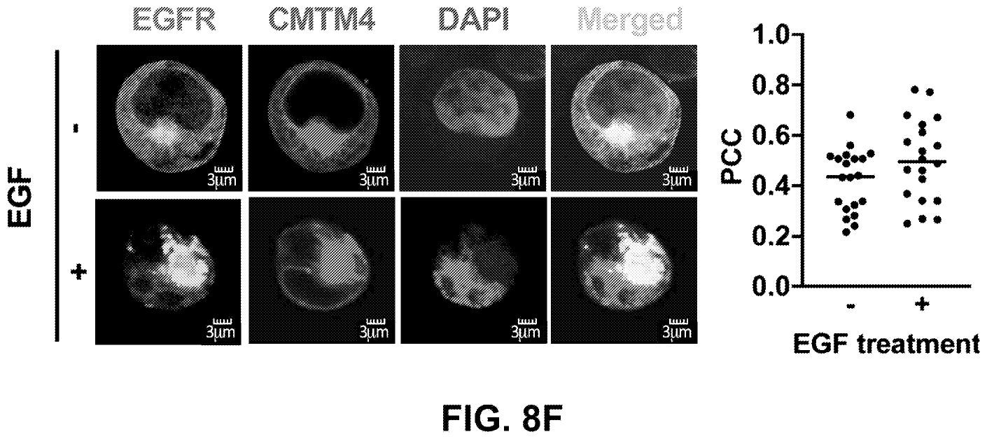

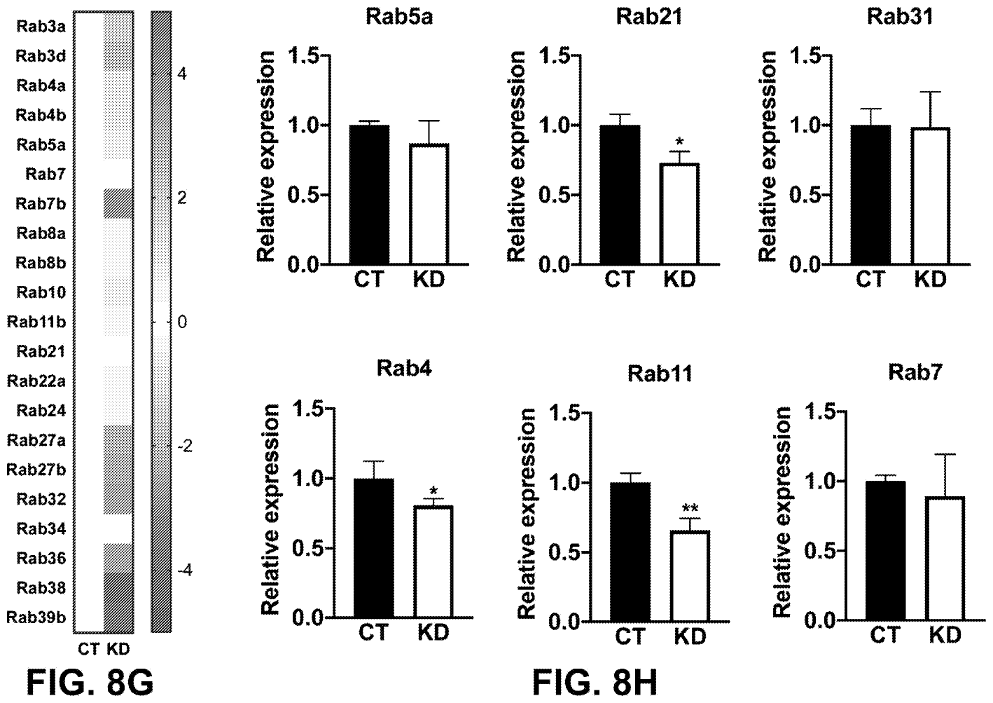

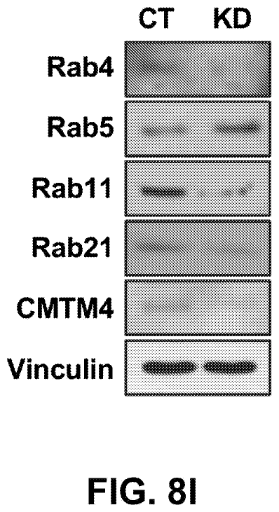

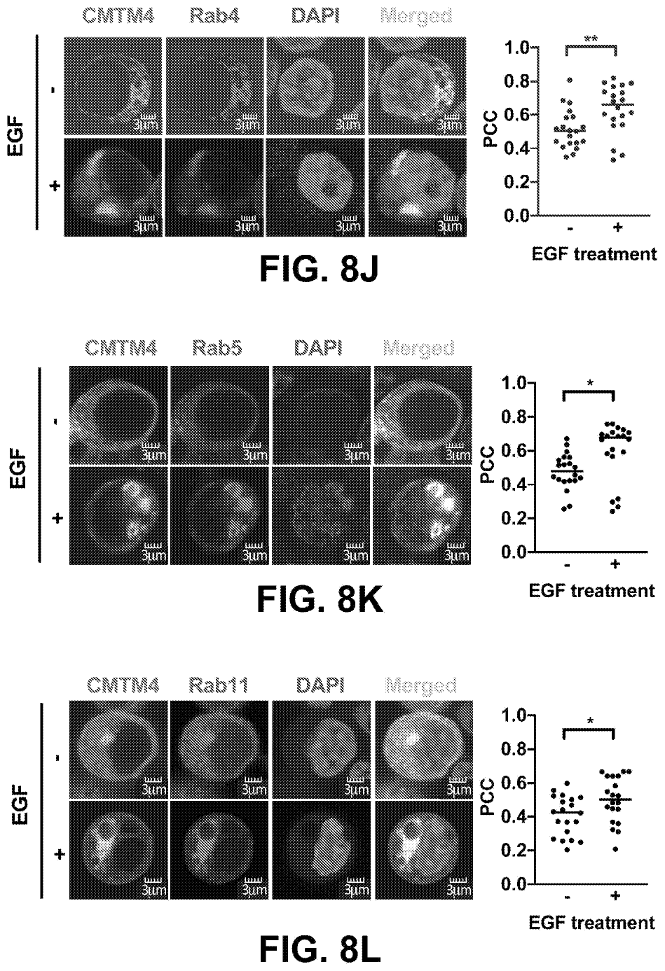

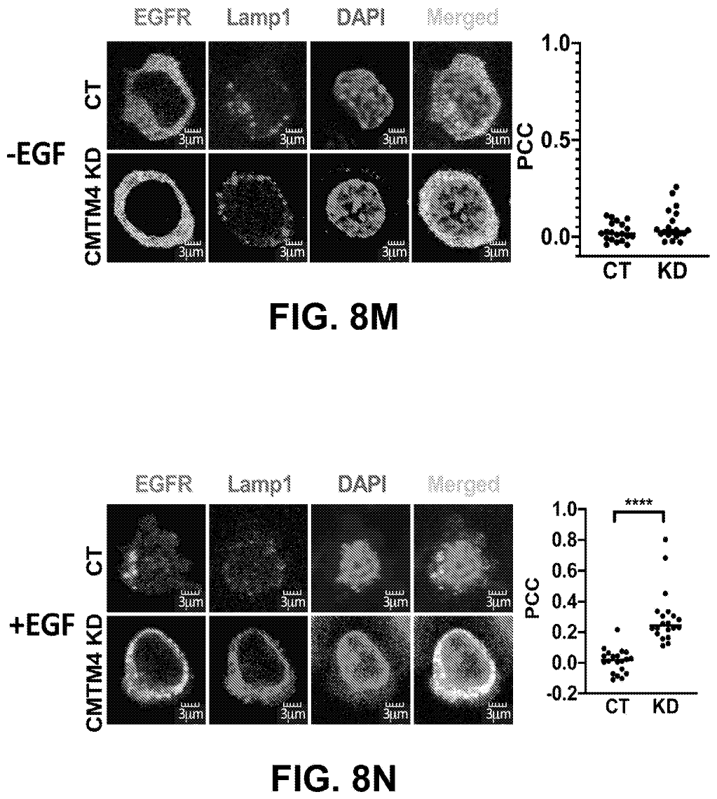

Since CMTM4 is a MARVEL transmembrane protein, in search of the mechanism of CMTM4 in EGFR regulation, we hypothesized that CMTM4 may affect the endocytic and recycling pathway. EGFR is known to maintain an auto-inhibitory conformation on the lipid rafts. Upon release from lipid rafts, EGFR is internalized and transported through the endocytic pathway. We noted that CMTM4 was expressed in cytosolic and non-raft membrane fractions as well as in lipid raft membranes (FIG. 8A). We further identified that CMTM4 KD induced an important increase in lipid rafts stained by the lipid raft tracer cholera toxin B-subunit (CT-B) (FIG. 8B). RNAseq data analysis revealed that gene expression regarding lipogenesis and cholesterol efflux was increased in CMTM4 KD cells but the genes related to lipid raft protein controlling signal transduction were decreased compared to control cells (FIG. 8C). Depletion of lipid raft with methyl-beta-cyclodextrin (MβCD) increased mRNA and protein levels of EGFR indicating that increased lipid raft in CMTM4 KD cells prevents the release of EGFR from lipid rafts (FIG. 8D, 8E).

(7) CMTM4 is Co-Localized with EGFR and Controls EGFR Recycling

We found that CMTM4 was co-localized with EGFR before the EGF stimulation and the colocalization signal was further enhanced by EGF stimulation (FIG. 8F). It has been known that EGFR endocytic trafficking is regulated by Rab4/5/11/21/22/31. The RNAseq data showed that CMTM4 KD reduced the expression of most Rab GTPase which coordinates endocytosis (FIG. 8G). We confirmed that CMTM4 KD significantly reduced the expression of Rab4/11/21 in mRNA and protein levels (FIG. 8H an 8I) indicating that CMTM4 KD may interfere with EGFR endocytosis and recycling. Confocal imaging of CMTM4 with different markers of intracellular vesicular transport compartment showed colocalization of CMTM4 with Rab5, early endosome marker, and Rab4/11, markers of recycling vesicles with EGF stimulation (FIGS. 8J, 8K, and *L). EGFR was not located in the lysosome of WT and CMTM4 KO cells without EGF stimulation (FIG. 8M). However, we have identified that EGFR was colocalized with lamp-1 (lysosome-associated membrane glycoprotein 1, lysosome tracker) in the CMTM4 KO cells while not in the control wild-type cells under EGF stimulation (FIG. 8N). These data indicate that CMTM4 controls EGFR signaling by protecting EGFR bound to the lysosome compartment and is involved in EGFR endocytosis and recycling process. Even when EGFR was overexpressed, it was transported to the lysosome and was degraded in CMTM4 KD cells. Thus, CMTM4 regulates the re-organization of lipid raft following EGFR release and the released EGFR activates Akt/mTOR/NF-κB signaling to produce inflammatory mediators and recycles through endosomal Rab proteins.

(8) CMTM4 siRNA Liposomes Inhibit Tumor Metastasis In Vivo

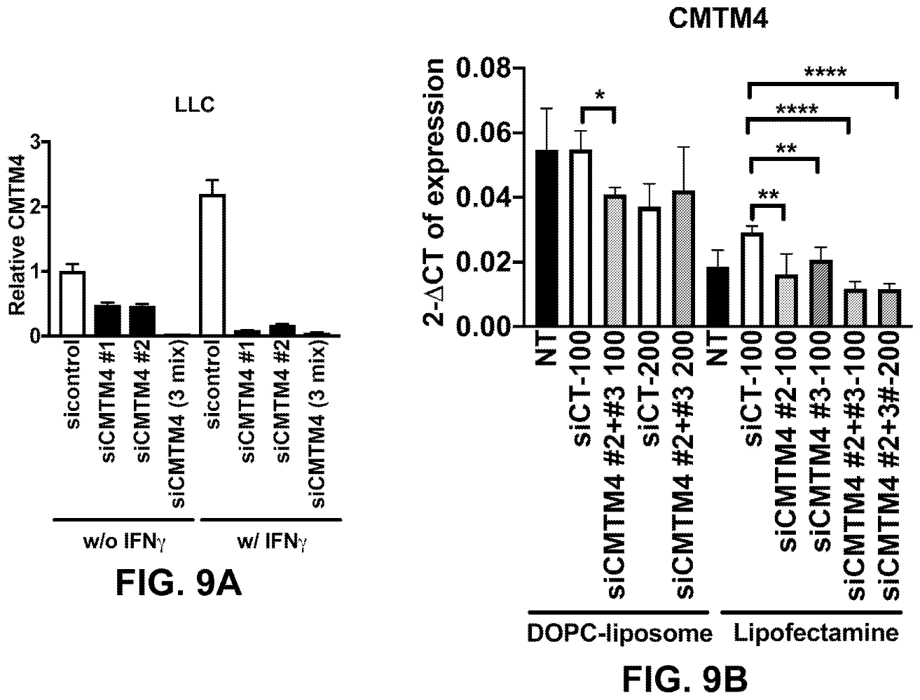

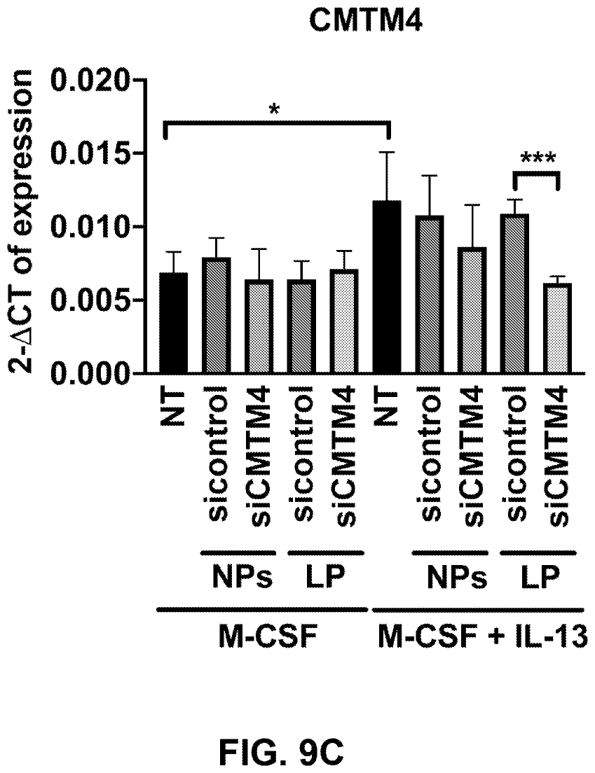

To target CMTM4 in tumor microenvironment, we generated three siRNA targeting CMTM4 expression. Transfection of siRNAs efficiently reduced expression of CMTM4 in LLC cells (FIG. 9A). Treatment of CMTM4 siRNA DOPC liposomes showed significant reduction of CMTM4 expression in LLC (FIG. 9B) and macrophages (FIG. 9C) whereas CMTM4 siRNA nanoparticles did not have an effect on CMTM4 expression in macrophages (FIG. 9C).

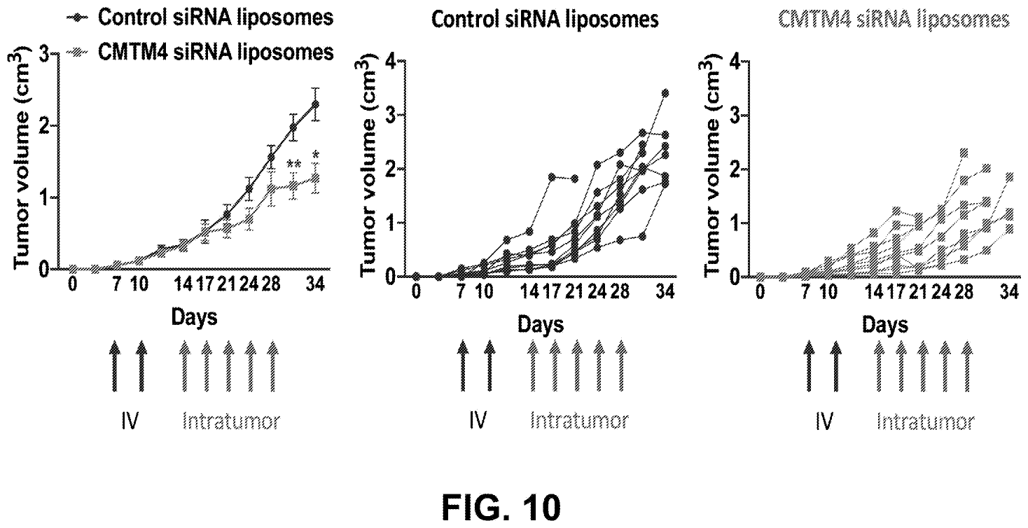

In order to evaluate the effect of CMTM4 siRNA liposomes on tumor growth, 4T1-implanted mice were treated with 30 ug control or CMTM4 siRNA liposomes every three days. CMTM4 siRNA liposomes treated mice indicated significantly retarded tumor growth in vivo (FIG. 10). Whereas control siRNA liposomes treated mice had necrosis on the tumors, CMTM4 siRNA liposomes treated mice did not have necrosis meaning reduced inflammation by CMTM4 siRNA liposomes.

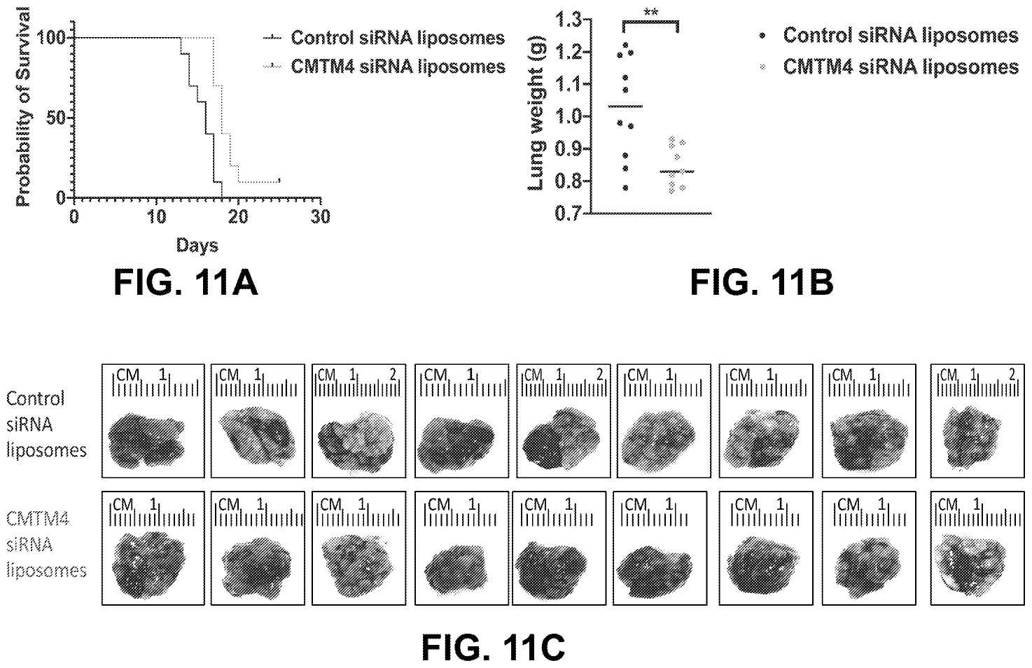

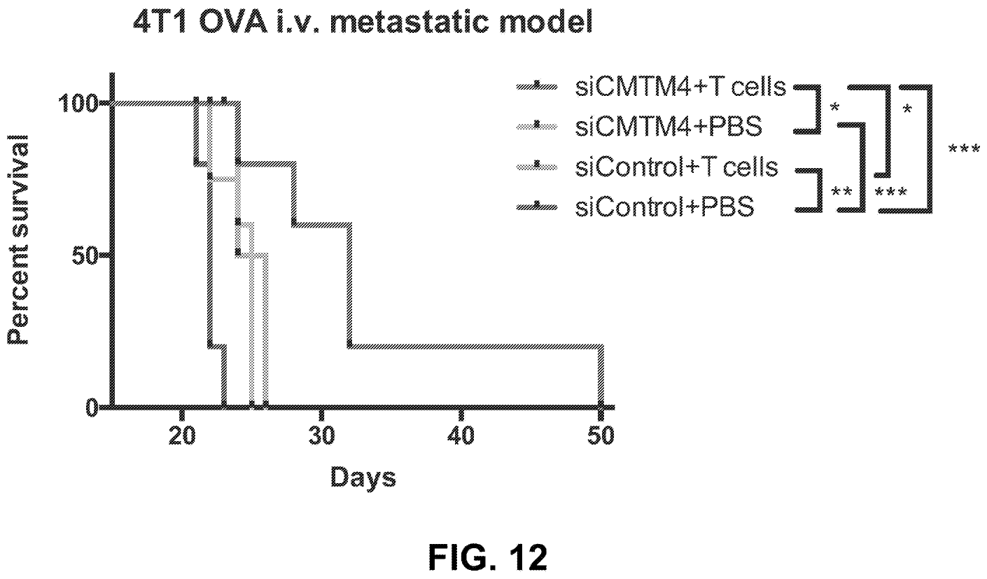

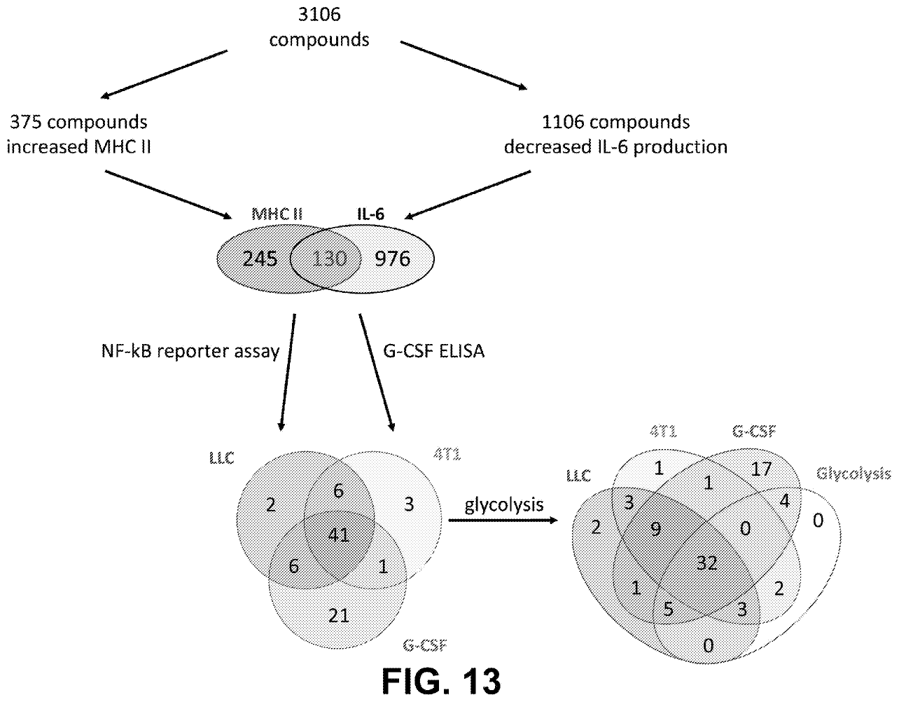

Next, we identified the effect of CMTM4 siRNA liposomes on tumor metastasis. Mice were injected 4T1 cells intravenously and 30 ug control or CMTM4 siRNA liposomes were treated to mice every three days after four days of cancer cell injection. Control siRNA liposomes-treated mice started to die earlier than CMTM4 siRNA liposomes-treated mice and survival rate from siRNA liposomes-treated tumor-bearing mice was significantly increased (FIG. 11A). When the mice died and lung weights were measured, the lungs from control siRNA liposomes-treated tumor-bearing mice were much heavier than the lungs from CMTM4 siRNA liposomes-treated tumor-bearing mice (FIG. 11B). Tumor nodules in the lungs also showed less in CMTM4 siRNA liposomes-treated tumor bearing mice (FIG. 11C). In order to see combination effect of CMTM4 siRNA liposomes with T cells, tumor-bearing mice were injected with 30 ug control or CMTM4 siRNA liposomes twice every week after four days of cancer cell injection and DO 11 T cells were transferred to the mice after 8 days of tumor implantation. We found that CMTM4 siRNA with T cells had significant synergistic effect on mouse survival (FIG. 12).

### (9) Screening of Compounds Targeting CMTM4

To identify compounds that can target CMTM4, we received about 3106 compounds from the Developmental Therapeutics Program, NCI/NIH. The initial screening was followed by the following assessments: 1) MHC 11 assay by high-throughput flow cytometry. 2) IL-6 production. 3) NF-kB promoter activity using CMTM4-expressing NF-kB/secreted embryonic alkaline phosphatase (SEAP) reporter stable cell lines using both LLC and 4T1 cells. 4) Inhibition of EGF-mediated G-CSF secretion from tumor cells. 5) Test glycolysis (FIG. 13). From the screening, we found that 32 compounds can target CMTM4 (Tables 2 and 3).

TABLE 2
32 compounds targeting CMTM4

| | Compound name |
|---|---|
| Plate 4878-131_Specimen_001_B11_B11_020.fcs | C29H38O4, CELASTROL |
| Plate 4865-131_Specimen_001_H10_H10_079.fcs | Ellipticine |
| Plate 13190761-043B_Specimen_001_E3_E03_042.fcs | C21H20F3NO6 |
| Plate 13190761-043B_Specimen_001_C3_C03_022.fcs | C36H48N2O12, Rhodomycin A |
| Plate 13190761-043B_Specimen_001_G3_G03_062.fcs | C16H24O5, Ovalicine subst. |
| Plate 13190761-043B_Specimen_001_C8_C08_027.fcs | C19H14O7, S-Methoxysterigmatocysin |
| Plate 13190761-043B_Specimen_001_G7_G07_066.fcs | C16H12O7 |
| Plate 13190761-043C_Specimen_001_C7_C07_026.fcs | C21H26N2O3, Tabernaemontanin |
| Plate 13190761-043C_Specimen_001_E10_E10_049.fcs | C27H34O10, Verrucarin A 9,10-epoxide |
| Plate 13190761-043C_Specimen_001_E5_E05_044.fcs | C10H12N4O5, Formycin B |
| Plate 13190761-043C_Specimen_001_G5_G05_064.fcs | C18H23NO6, Swazine |
| Plate 13190761-043C_Specimen_001_A4_A04_003.fcs | C28H33N3O•ClH, Butylcycloheptylprodiginine |
| | hydrochloride |
| Plate 13190761-043C_Specimen_001_G7_G07_066.fcs | C16H12O7 |
| Plate 23190761-043C_Specimen_001_C2_C02_021.fcs | C12H13N5O4, Toyocamycin |
| Plate 13190761-043B_Specimen_001_G10_G10_069.fcs | C18H22N2O4•C6H8O7, Quinocarcin monocitrate |
| Plate 4883-67_Specimen_001_H9_H09_078.fcs | C17H19N3O4, Anthramycin methyl ether |
| Plate 13190761-043A_Specimen_001_G11_G11_070.fcs | C23H28N2O5 |
| Plate 19190763-043A_Specimen_001_A11_A11_010.fcs | C11H13NO2•BrH |
| Plate 13190761-043A_Specimen_001_E11_E11_050.fcs | C21H24N2O3, Strychninic acid |
| Plate 13190761-043A_Specimen_001_B3_B03_012.fcs | C21H22N2O3, Pseudostrychnine |
| Plate 13190761-043A_Specimen_001_E5_E05_044.fcs | C20H18N2O5•Na, Camptothecin sodium |
| Plate 13190761-043A_Specimen_001_C7_C07_026.fcs | C18H16O8, Centaureidin |
| Plate 13190761-043A_Specimen_001_A9_A09_008.fcs | C10H14O3, Ramulosin |
| Plate 13190761-043A_Specimen_001_E9_E09_048.fcs | C9H16N2O6, Tetrahydrouridine |
| Plate 13190761-043A_Specimen_001_C9_C09_028.fcs | C10H14O3 |
| Plate 13190761-043B_Specimen_001_A4_A04_003.fcs | C19H26N2•C4H6O6 |
| Plate 13190761-043A_Specimen_001_E7_E07_046.fcs | C15H10N2O6, Lomondomycin |
| Plate 13190761-043A_Specimen_001_A5_A05_004.fcs | C14H25N3O9, Kasugamycin |
| Plate 13190760-043A_Specimen_001_F8_F08_057.fcs | C4H6O2, CROTONIC ACID |
| Plate 13190761-043C_Specimen_001_A5_A05_004.fcs | C21H26N2O3, Pseudoyohimbine |
| Plate 13190761-043A_Specimen_001_G3_G03_062.fcs | Fumitremorgin C |
| Plate 13190760-043A_Specimen_001_E8_E08_047.fcs | C26H28O5, Glycrrhizol A |

TABLE 3

| | | MHC II | IL-6 | NF-kB | NF-kB | G-CSF | Glycol- | p-Akt/ | |
|---|---|---|---|---|---|---|---|---|---|
| | Compound name | (%) | (%) | (%)-LLC | (%)-4T1 | (%) | ysis | p-mTOR | CMTM4 |
| 2 | Ellipticine | 208.3695652 | −15.7288 | 40.9190372 | 39.8926655 | 7.711589 | Inhibit | Decrease | No difference |
| 4 | C54H78N2O17, | 360.4651163 | −12.2043 | 37.63676149 | 40.07155635 | 1.147141 | Inhibit | Decrease | Decrease |
| | Lobophorin F | | | | | | | | |
| 6 | C19H14O7, 5- | 341.5282392 | −11.581 | 35.2297593 | 38.64042934 | 1.533285 | Inhibit | Decrease | No difference |
| | Methoxysterigmatocysin | | | | | | | | |
| 7 | C16H12O7, Isorhamnctin | 290.1993355 | −11.581 | 59.73741794 | 34.16815742 | 49.80128 | Inhibit | Decrease | No difference |
| 9 | C27H34O10, Verrucarin A | 280.2325581 | −10.1997 | 36.10503282 | 41.32379249 | −0.01129 | Inhibit | Decrease | No difference |
| | 9,10-epoxide | | | | | | | | |
| 10 | C10H12N4O5, Inosine | 456.6445183 | −9.43709 | 41.35667396 | 40.4293381 | 0.374853 | Inhibit | Decrease | No difference |
| | | | | | | | | (Slow) | |
| 11 | C18H23NO6 | 304.8172757 | −6.7679 | 46.38949672 | 50.80500894 | 2.691717 | Inhibit | Decrease | No difference |
| 12 | C25H33N3O•ClH | 426.2458472 | −6.00527 | 53.61050328 | 45.7960644 | 4.236293 | Inhibit | Little | Decrease |
| | | | | | | | | Decrease | |
| 13 | C16H12O7, Rhamnetin | 228.9036545 | −5.24264 | 40.26258206 | 41.68157424 | 0.760997 | Inhibit | Decrease | No difference |
| 14 | C12H13N5O4, Toyocamycin | 329.7342193 | −3.71739 | 44.42013129 | 45.61717352 | 7.711589 | Inhibit | Decrease | No difference |
| 16 | C17H19N3O4, Anthramycin | 279.6128013 | −1.98239 | 53.61050328 | 58.31842576 | 10.4146 | Inhibit | Decrease | No difference |
| | methyl ether | | | | | | | | |
| 20 | C21H22N2O3, Paquinimod | 520.5980066 | −0.80902 | 32.60393873 | 46.86940966 | 3.464005 | Inhibit | Decrease | No difference |
| 21 | C20H18N2O5•Na, | 372.5913621 | −0.80902 | 31.72866521 | 42.21824687 | −0.39743 | Inhibit | Decrease | No difference |
| | Camptothecin sodium | | | | | | | | |
| 22 | C18H16O8, Rosmarinic Acid | 488.0398671 | −0.12325 | 33.04157549 | 39.89266547 | 0.760997 | Inhibit | Decrease | No difference |
| 23 | C10H14O3 | 340.5315615 | −0.12325 | 34.35448578 | 42.03935599 | 3.850149 | Inhibit | Decrease | No difference |
| 24 | C9H16N2O6, | 317.7740864 | −0.12325 | 35.2297593 | 40.07155635 | 0.374853 | Inhibit | Decrease | No difference |
| | Tetrahydrouridine | | | | | | | | |
| 25 | C10H14O3, Mephenesin | 367.7740864 | 0.562528 | 35.2297593 | 40.60822898 | 2.691717 | Inhibit | Decrease | No difference |
| 26 | C23H32N2O6 | 1814.451827 | 0.886166 | 56.89277899 | 55.63506261 | 10.02845 | Inhibit | No difference | Decrease |
| 27 | C15H10N2O | 273.089701 | 1.248304 | 35.66739606 | 40.96601073 | −0.39743 | Inhibit | Decrease | Decrease |
| 28 | C14H25N3O9, Kasugamycin | 483.3887043 | 1.934079 | 46.82713348 | 44.18604651 | 66.40547 | Inhibit | Decrease | No difference |
| 29 | C4H6O2, CROTONIC ACID | 1066.573816 | 4.437502 | 37.19912473 | 40.4293381 | 1.147141 | Inhibit | Decrease | No difference |
| 30 | C21H26N2O3, Vincamine | 230.2325581 | 5.815462 | 48.14004376 | 50.62611807 | 6.939301 | Inhibit | No difference | Decrease |
| 31 | Fumitremorgin C | 5071.262458 | 10.84916 | 42.88840263 | 49.73166369 | 6.939301 | Inhibit | Decrease | No difference |

### b) Discussion

The complex interaction between the immune system and the tumor remains a major obstacle in the development of effective anti-tumor therapies. Effectively counteracting or neutralizing tumor-associated inflammation will result in reprogramming or recovery of skewed immune responses within the tumor microenvironment. Alternatively, targeting immune suppressive cells will quell a cascade of tumor-promoting events, thereby ultimately favoring cancer rejection. In this study, we showed that CMTM4 controlled tumor growth by modulating the tumor-associated inflammation and leukocyte infiltration through EGFR expression and activation. Although CMTM4 was shown to be highly expressed in various cancer types, its functional roles in tumor progression and establishment of the tumor microenvironment have not been fully investigated. Human carcinoma tissues and murine tumor cell lines express high levels of CMTM4 (FIG. 2A-D, G). Interestingly, higher stage of cancer patient tissues had higher CMTM4 expression than the lower stage of human cancer patient tissues in lung adenocarcinoma (FIG. 2C) and triple-negative basal breast cancer cell lines had higher CMTM4 expression compared to luminal type cell lines (FIG. 2D) indicating that CMTM4 is associated with an aggressive phenotype of cancer. We further demonstrated that CMTM4 can be used as an independent poor prognostic factor for survival in patients with certain cancer types since the corresponding patient group with higher expression of CMTM4 showed a significantly reduced survival rate compared to the group with lower CMTM4 expression (FIG. 2E, F, Table 1).

Ting Li et al. showed that CMTM4 was downregulated in tumor cells and functioned as a tumor suppressor gene. However, the reduced expression of CMTM4 was limited to certain cancer types, including glioblastoma, neuroblastoma, and clear cell renal cell carcinoma. Overall tumor tissues, including breast cancer, lung adenocarcinoma, lung squamous cell carcinoma, melanoma, ovarian, pancreatic, and prostate cancers, had higher CMTM4 expression levels, which is consistent with our data (FIG. 1A, 2B). Therefore, these data indicate that CMTM4 may have different roles depending on the cancer type and the role of CMTM4 in each type of cancer needs to be delineated. In contrast to a previous finding that CMTM4 inhibited HeLa cell growth by inducing G2/M phase arrest, we found that CMTM4 knockdown did not affect the proliferation of tumor cells in vitro (FIG. 3C). Although closely associated with the tumor suppressor locus 16q22.1, which is frequently deleted in multiple tumors, CMTM4 expression is maintained in carcinoma tissues. By contrast, CMTM3, which is located in the same region, is silenced in carcinomas. Therefore, unlike other CMTM family members, CMTM4 might not have a tumor suppressor role. Interestingly, the inhibition of CMTM4 expression in cancer cells reduced tumor growth in vivo but not in vitro (FIG. 4A, 4B). Therefore, these findings indicate that CMTM4 can be an important regulator in the establishment of the suppressive and pro-tumor microenvironment.

In the course of tumorigenesis, the tumor microenvironment is gradually altered in favor of tumor growth. Several mediators, such as cytokines and chemokines secreted by cancer cells, recruit immune cells to the tumor site to promote tumor development. Therefore, immune evasion by cancer cells is crucial during oncogenesis and is considered a hallmark of cancer. Our data indicate that CMTM4 KD reduced infiltration of major immunosuppressive cells, PMN-MDSCs and Treg cells, to tumors. Furthermore, monocytic-MDSCs had M1-like anti-tumor functional phenotypes in CMTM4 KO tumor-bearing mice. Reducing the immunosuppressive effects of immune cells has emerged as a promising approach to improve the efficacy of anti-tumor therapeutics. Direct targeting of MDSCs in cancer patients has several limitations, including the diverse phenotypes and the lack of available specific surface markers. Therefore, CMTM4 represents a novel target for intervention with the tumor-promoting activities of MDSCs without direct targeting of MDSCs.

The attractive targetable pathways include genes that regulate multiple cytokines or chemokines, especially activation of the NF-κB and mTOR pathways, depletion or reprogramming of cancer-promoting tumor-associated immune cells, and blockade of suppressive mechanisms by myeloid cells. Our results indicate that CMTM4 controls oncogenic pathways, RTKs, to alter the immune system. RTKs have been identified to be the most frequent oncogenic driver involved in cancer cell proliferation, survival, and metastasis. Targeting RTK can inhibit tumor growth and synergize the efficacy of anti-cancer therapeutics. CMTM4 KD decreased the expression and activation of multiple RTKs (FIG. 6A-D). CMTM4 KD reduced activation of EGFR/Akt/mTOR and NF-κB pathways (FIG. 6). Cancer patients harboring EGFR show poor responses and side effects to tyrosine kinase inhibitors. Regulation of CMTM4 may benefit through effective inhibition of RTKs including EGFR. We also found that CMTM4 KD diminished the expression of multiple cytokines and chemokines responsible for MDSC accumulation (FIG. 6L). Notably, G-CSF production was significantly diminished in response to EGF in CMTM4 KD cells, consistent with that oncogenic RTK-mTOR pathway has been known to drive MDSC accumulation through G-CSF. Our data demonstrate that CMTM4 controls tumor cell-intrinsic oncogenic pathways determining the tumor's capacity to accumulating MDSC.

We further identified CMTM4 regulates lipid raft and is associated with Rab GTPases involved in EGFR endocytosis and recycling (FIG. 8). CMTM4 KD suppressed expression of Rab4/11/21 which controls EGFR endocytosis and recycling (FIG. 8G, 8H, 8I). Although Rab21 was decreased in CMTM4 KD cells among Rab proteins controlling endocytosis, Rab5 and Rab31 were not reduced by CMTM4 KD indicating that CMTM4 KD may not inhibit endocytosis. However, Rab4 and Rab21, which are involved in recycling, were significantly diminished in CMTM4 KD cells (FIG. 8H, 8I) indicating CMTM4 is associated with Rab4 and Rab21 to regulate EGFR recycling to the membrane. Interestingly, overexpression of EGFR in CMTM4 KD cells was colocalized with lamp1 in response to EGF whereas EGFR is not colocalized with lamp1 in WT cells. It reveals that CMTM4 KD cells have reduced EGFR expression but even leftover EGFR may be degraded in lysosomes in CMTM4 KD cells (FIG. 8M, 8N). Lots of Rab proteins are considered as a predictive marker for cancer treatment and potential candidate targets for enhancing therapeutic efficacy. Studies are underway to further characterize which CMTM4 modulates tumorigenic potential by controlling Rab expression and activation.

In summary, our study reveals an exciting function of CMTM4 in the tumor microenvironment. The central regulatory role played by CMTM4 KD in tumor cells relates to the establishment of a suppressive tumor microenvironment leading to reduced tumor growth. Pharmacological targeting of CMTM4, therefore, may reduce tumor inflammation, thereby inhibiting tumor growth. Our findings indicate that CMTM4 is an intrinsic novel molecule that can be targeted to convert the tumor microenvironment from an immune-suppressive one to an immune conducive one.

### c) Materials and Methods

### (1) Plasmids and Reagents

Constructs for EGFR, Rab4, 5, 11, and Lamp-1 were purchased from Addgene. Alexa Fluor 647-conjugated cholera toxin B (Life Technology) was used to label lipid raft.

### (2) Immunohistochemistry and Immunostaining

Primary human carcinoma samples were obtained from the Biorepository Core, Icahn School of Medicine at Mount Sinai. Paraffin-embedded sections were stained with polyclonal antibodies against CMTM4 (Atlas Antibodies, Sweden). The endothelial cell layer of the mouse tumor was stained using rabbit anti-mouse CD31 followed by incubation with Texas Red conjugated goat anti-rabbit IgG antibody (Vector Laboratories). Slides were examined and imaged using a confocal scanning microscope (Zeiss) or FluoView TM3000 at Houston Methodist Research Institute's Advanced Cellular and Tissue Microscopy Core Facility.

(3) siRNA Transfection

siRNA-mediated gene silencing was performed by using the retroviral expression vector pSIREN-RetroQ (Clontech Laboratories Inc., Mountainview, CA) to express small hairpin RNA (shRNA). The CMTM4-specific insert consisted of a 19-nt sequence (CTTGATTAGAAGGACGGTT) separated by a non-complementary spacer from the reverse complement of the same 19-nt sequence to form the shRNA duplex, referred to as pSR si-CMTM4. A control vector was used as a control.

### (4) Western Blot, Co-Immunoprecipitation (Co-IP), and Proteome Profiler RTK Array

Protein samples from cells were separated on sodium dodecyl sulfate (SDS)-polyacrylamide gels and transferred to PVDF membranes. The membranes were blocked in 4% skim milk solution, incubated with the appropriate antibody, and subsequently incubated with a secondary antibody conjugated with horseradish peroxidase. The antibodies for p-NF-κBp65, NF-κBp65. EGFR, PTEN, Akt, p-Akt, mTOR, p-mTOR, S6, p-S6, Rab4, Rab5a, Rab11, Tubulin, actin, vinculin, and CMTM4 were purchased from Cell Signaling Technology, Inc. (Beverly, MA) and the antibody for Rab21 was purchased from Santa Cruz Biotechnology. The immunoreactive bands were visualized using the ECL system (Thermo Scientific). Control and CMTM4 KD cell lysates were analyzed using the proteome profiler mouse phospho-receptor tyrosine kinase array kit (R&D Systems) following the manufacturer's instructions. For immunoprecipitation, Dynabeads His-Tag isolation and pulldown kit (Life Technologies) was used. The pull-down samples were subjected to immunoblot assays.

### (5) Lipid Raft and Non-Raft Protein Isolation

Lipid raft and non-raft proteins were isolated using the UltraRIPA kit for lipid raft (Diagnocine, Hackensack, NJ) according to the manufacturer's instruction.

### (6) Isolation and Sorting of Myeloid-Derived Suppressor Cells (MDSCs)

C57BL/6 mice were injected subcutaneously with 5×105 Lewis lung carcinoma (LLC) cells. Mice were sacrificed when tumors reached 1.5×1.5 cm2. Splenocytes and bone marrow were processed to single-cell suspensions. MDSCs were enriched by Percoll density gradient (GE Healthcare, UK). Fraction 2 cells were stained, in the presence of FcR blocking Ab, with anti-Ly6C and anti-Gr-1 antibodies, followed by sorting into monocytic (Gr-1LoLy6cHi) and polymorphonuclear (PMN) (Gr-1HiLy6cLo) populations via the MoFlo XPD High-Speed Cell Sorter (Beckman Coulter).

### (7) In Vivo Tumor Growth Rate Comparisons

5×105 CT or CMTM4 KD tumor cells were inoculated into the flanks of BALB/c (4T1 or MCA26 cells) or C57BL/6 (B16 or LLC cells) mice. Tumor sizes were measured every 2 days.

### (8) Suppression Assays

The suppressive activity of MDSCs was assessed in peptide-mediated proliferation assays of TCR transgenic T cells. Briefly, 105 splenocytes from OT-II mice were cultured in the presence of OVA peptides (1 μg/mL) and serial dilutions of MDSCs in 96-well plates (Corning). Proliferation was determined based on [3H]-thymidine uptake after 48 hours of stimulation.

### (9) Antibodies and Flow Cytometry

Anti-Ly6C, anti-Ly6G, anti-Gr-1, anti-CD11b, anti-CD45, anti-CD206, anti-iNOS, and isotype-matched mAbs were purchased from eBioscience (San Diego, CA). Anti-Arginase was purchased from R&D Systems (Minneapolis, MN). Flow cytometry was performed using FACSCanto IT (BD Biosciences) instruments and data was analyzed using Flowjo software (Flowjo, LLC).

### (10) Adoptive Transfer Experiments

7×104 CT or CMTM4 KD LLC cells were inoculated into the livers of CD45.1 C57BL/6 mice. MDSCs were harvested from these mice and sorted into monocytic and PMN populations. Sorted MDSCs were then adoptively transferred via tail vein injection (5×106 cells per mouse) into MaFIA mice that had been hepatically implanted with control or CMTM4 KD LLC tumor cells 14 days earlier. Before adaptive transfer, tumor-bearing MaFIA mice were depleted of CD115+ cells. Mice were sacrificed 4 days after adoptive transfer. Spleen, bone marrow, and tumor were harvested and the percentage, number, and phenotype of MDSCs were assessed by flow cytometric analysis.

### (11) Real-Time PCR

RNA was isolated using Trizol (Invitrogen; Carlsbad, CA) per the manufacturer's specifications. cDNA was synthesized from 1 μg of total RNA using M-MLV reverse transcriptase (Promega, Madison, WI) and qPCR was performed in 384-well plates using FastStart SYBR Green Master Mix (Roche, Mannheim, Germany) on a ViiA™ 7 real-time PCR system (Applied Biosystems, Foster City, CA).

### (12) ELISA Assay

Secretion of G-CSF was determined using ELISA kits according to the manufacturer's instructions (Life Technologies).

### (13) TCGA Data Analysis

For the different stages in lung adenocarcinoma, R package (TCGABiolinks) was used to pull TCGA clinical data and RNAseq data. Patients were separated by overall stage and looked at their CMTM4 gene expression (FPKM).

For the tumor vs. normal CMTM4 gene expression in various cancers, boxplot analysis was used looking only at CMTM4 from the GEPIA2 web server. The boxplot analysis takes data from TCGA and GTEx (Genotype Tissue Expression) databases.

### (14) Survival Analysis

The data were downloaded from the PROGgeneV2 prognostic database. The lung cancer dataset GSE26939 includes human lung adenocarcinoma mRNA expression and gene mutations from 115 samples. The breast cancer dataset GSE37751 includes molecular profiles of 60 human breast cancer samples and their association with tumor subtypes and disease prognosis (Affymetrix). The adrenal cancer data set GSE33371 includes beta-catenin status effects in human adrenocortical carcinomas (33 samples) and adenomas (22 samples). Normal adrenal cortex (10 samples) and GSE19776 include adrenocortical carcinoma gene expression profiling of 21 samples. The brain cancer dataset GSE7696 includes glioblastoma from a homogenous cohort of treated patients enrolled in a clinical trial (76 samples) and GSE4271 includes molecular subclasses of high-grade glioma sorted by prognosis, disease progression, and neurogenesis (76 samples). The neuro-endocrine cancer dataset GSE62564 includes 497 samples. Myeloid leukemia (156 samples) and head and neck squamous cell carcinoma (290 samples) datasets were obtained from TCGA. To investigate the prognostic value of CMTM4 expression, the samples were partitioned into two groups using median CMTM4 expression levels, and log-rank tests were performed to compare the Kaplan-Meier curves of the two patient groups.

### (15) Transcriptome Analysis

LLC control and CMTM4 KD cells were subjected to RNAseq analysis. RNA sample quality and quantity were assessed using Nanodrop, agarose gel electrophoresis, and Agilent 2100. DNA library preparation was performed using NEBNext Ultra DNA Library Prep Kit for Illumina (New England Biolabs, Ipswich, MA, USA). Sequencing was performed on the Illumina Hiseq X Ten at 150 bp paired end reads with 20 M read depth. All samples had Q30 >90%. Both library preparation and sequencing were performed by Novogene (Sacramento, California). Differential gene analysis was performed using the HISAT2-Cufflinks workflow. Gene ontology enrichment analysis and visualization were performed. The online data analysis tool Ingenuity pathway analysis (IPA) was used for genes that had a p-value of <0.05 and a ≥2-fold change (FC) difference between control and CMTM4 KD cells. Core analysis was run on this data set to determine the pathways most affected by the loss of CMTM4.

### (16) Reverse Phase Protein Array (RPPA)

LLC control and CMTM4 KD cells were subjected to RPPA experiments. RPPA data were generated by the RPPA core facility at the MD Anderson Cancer Center.

### (17) Statistical Analysis

Statistical analyses were performed using Student's t-test or one-way ANOVA in GraphPad Prism 9. The results are presented as mean±SD. A P value of <0.05 was considered to be statistically significant.

### 2. Example 2: Inflammatory Microenvironment Deceives the Therapeutic Outcome by CMTM4 Polarized Macrophage

### a) Results

### (1) CMTM4 can Control Macrophage Polarization

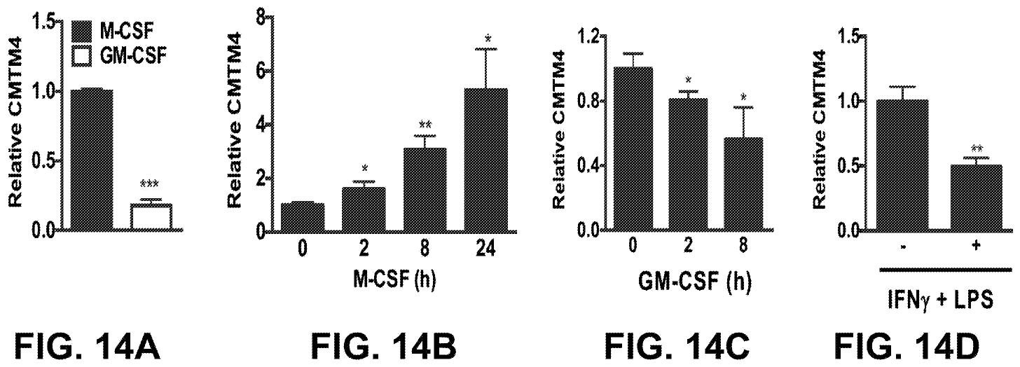

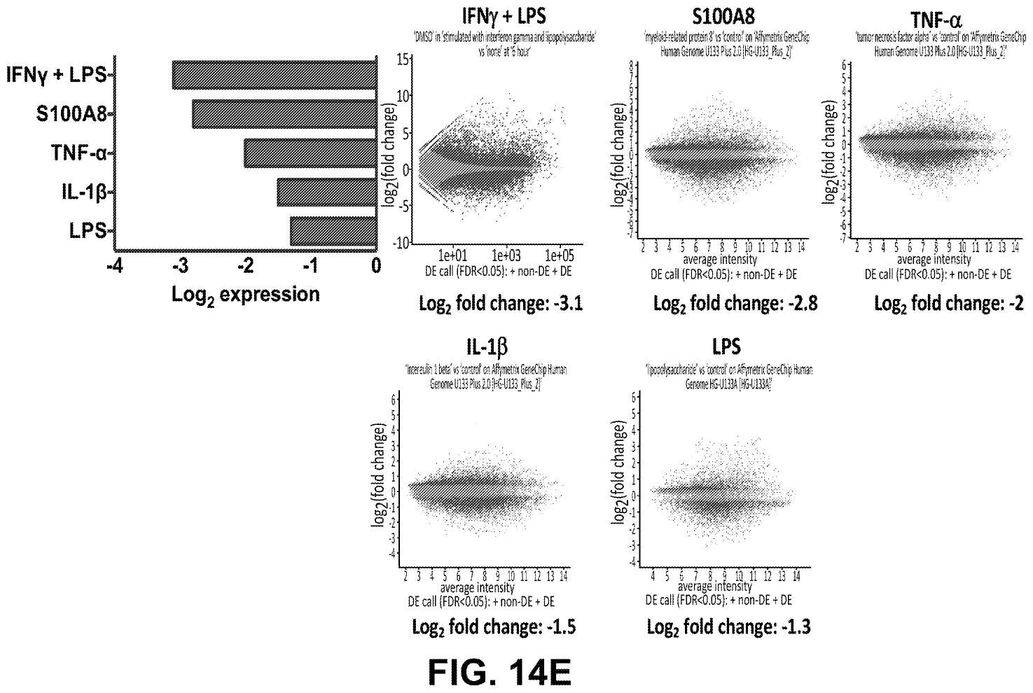

Since CMTM4 and CMTM6 have been reported to control PD-L1 expression, which is more toward the M2 macrophage markers, we have further evaluated the CMTM4 expression under the M1 vs. M2 differentiation conditions. CMTM4 expression levels were evaluated in myeloid cells differentiated with M2 (M-CSF) vs. M1 (GM-CSF) differentiation, respectively. The CMTM4 expression was higher in the myeloid cells cultured in M-CSF condition as compared to the cells cultured in GM-CSF condition (FIG. 14a). The expression of CMTM4 was gradually increased depending on the time of M-CSF treatment in bone marrow myeloid Ly6C+ cells whereas the expression of CMTM4 was decreased by GM-CSF treatment in a time-dependent manner (FIGS. 14b and 14c). M1 maturation polarizing stimulus, IFNγ, and LPS, also further decreased CMTM4 expression in macrophages (FIG. 14D). We also confirmed that human monocytes treated with LPS, IL-1β, IFNγ, TNF-α, and S100A8 had diminished expression of CMTM4 from microarray data downloaded from expression atlas (FIG. 14E). Since CMTM4 has differential expression in M1 and M2 macrophages, we hypothesize that CMTM4 may regulate the fate of macrophage differentiation.

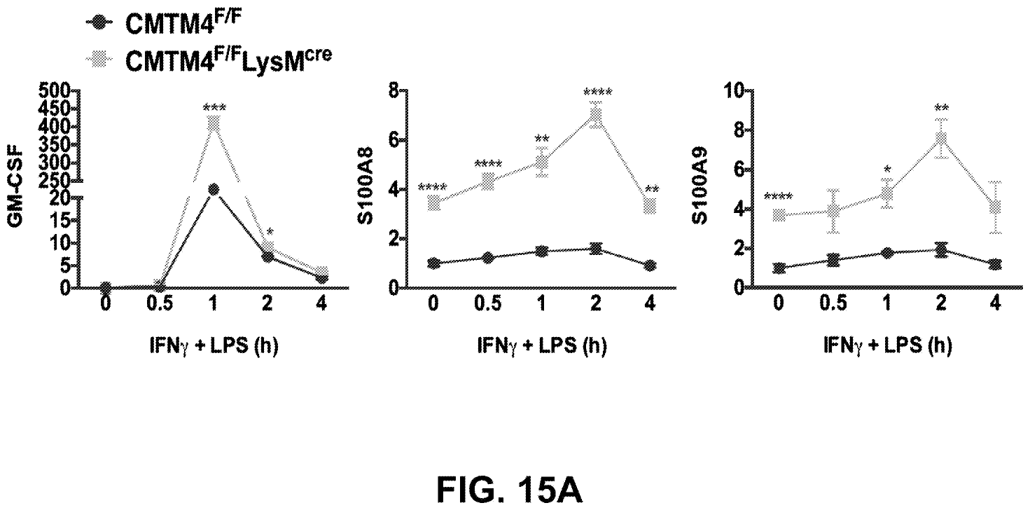

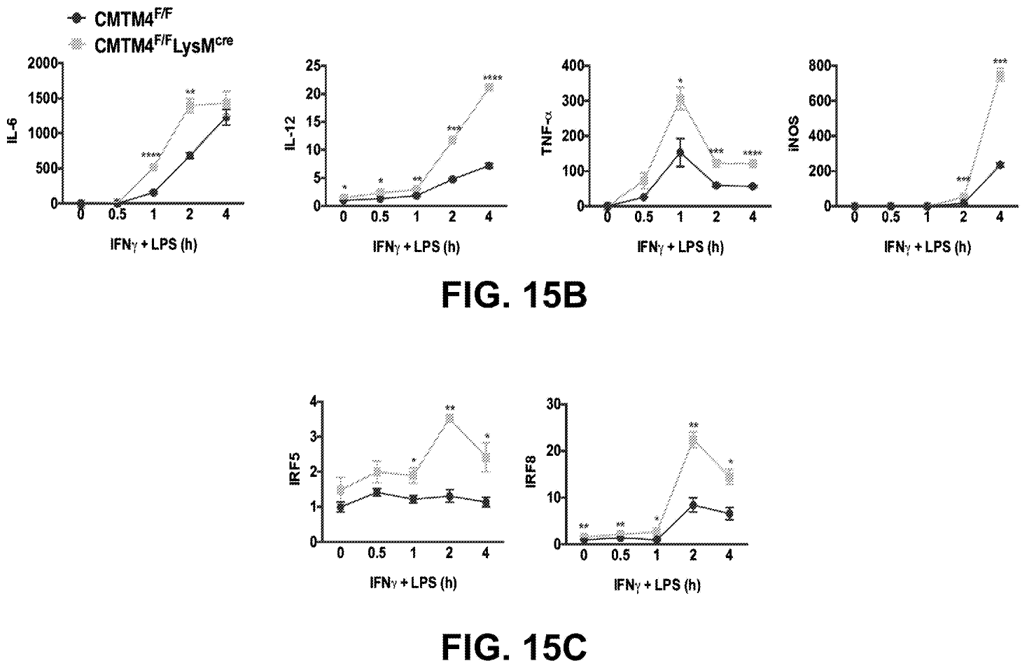

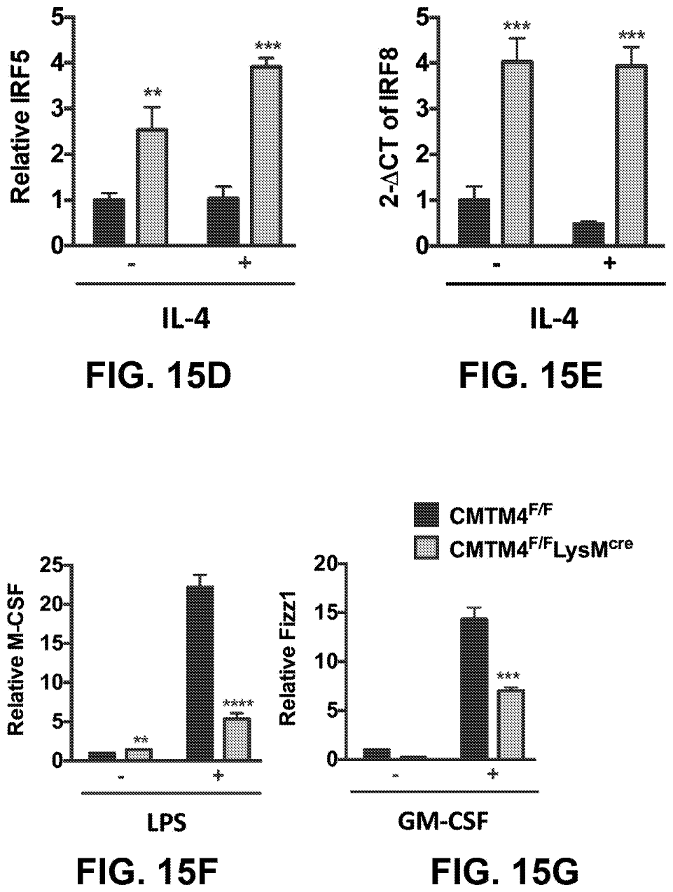

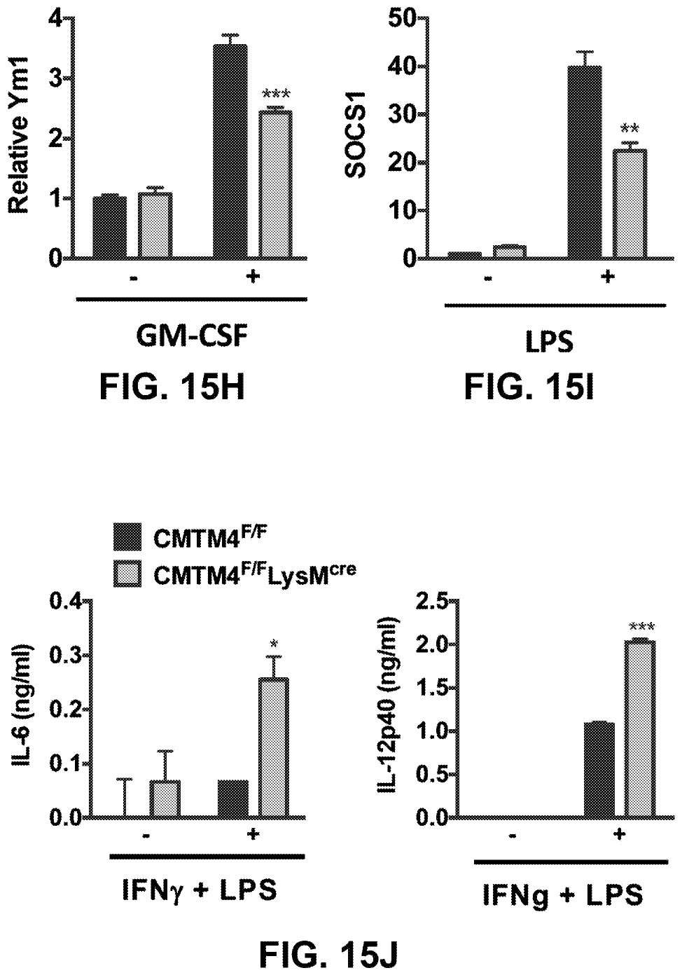

We further examined whether CMTM4 directly controls macrophage polarization. We have generated mice with lysozyme M-cre (LysM cre) and CMTM4-flox alleles to knock out (KO) of the CMTM4 gene in myeloid cells. There were no changes in the number and frequency of immune cells in the bone marrow and spleen. The expression of GM-CSF, S100A8, S100A9, IL-6, IL-12, TNF-α, and iNOS were higher in CMTM4-deficient macrophages. Higher expression of inflammatory cytokines in CMTM4-deficient macrophages was further increased after stimulation with IFNγ and LPS (FIGS. 15A and 15B). Even when the macrophages were stimulated with IL-4, the expression of M1 transcriptional factor e.g. LRF5 and IRF8 were maintained at significantly higher levels in CMTM4-deficient macrophages than control mice (FIGS. 15D and 15E). The expression of M-CSF, Fizz1, Ym1, and SOCS1, which are expressed in M2 macrophages, was lower in CMTM4-deficient macrophages compared to WT macrophages (FIGS. 15F, 15G, and 15H). The production of IL-6 and IL-12 were significantly enhanced in CMTM4-deficient macrophages in response to IFNγ and LPS (FIG. 15J). Collectively, CMTM4 KO can promote the M1 macrophages differentiation especially in response to cytokine stimulation. Together, these data indicate that CMTM4 can be the key regulator in the control of macrophage differentiation.

(2) CMTM4 KO Enhances Activation of M1 Activation Signaling Pathway while Suppresses M2 Differentiation in Macrophages

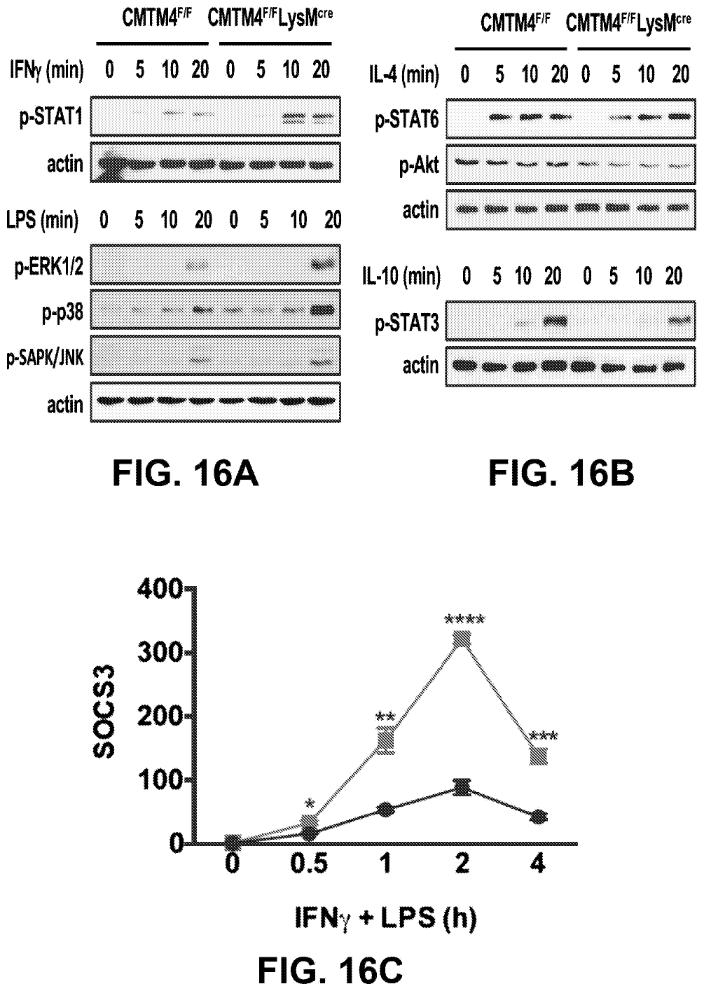

To understand how CMTM4 regulates macrophage polarization, we evaluated molecular pathways involved in this process. CMTM4 KO macrophages showed increased activation of STAT1, ERK1/2, p38, and SAPK/JNK with IFNγ or LPS stimulus (FIG. 16A). Furthermore, CMTM4 KO inhibits STAT3, STAT6, and PI3K/Akt signaling in M2 macrophages with IL-4 or IL-10 stimulus while increased the expression of IRF5 and IRF8, which promote M1 macrophage polarization, as well as had increased expression of SOCS3 with IFNγ and LPS stimulation (FIGS. 15C and 16B). Therefore, these data support that CMTM4 enhances activation of M2 polarizing signaling whereas it reduces activation of M1 polarizing signaling in macrophages.

### (3) CMTM4 KO Alters Defense, Inflammation, Cytokine Production Response Pathways

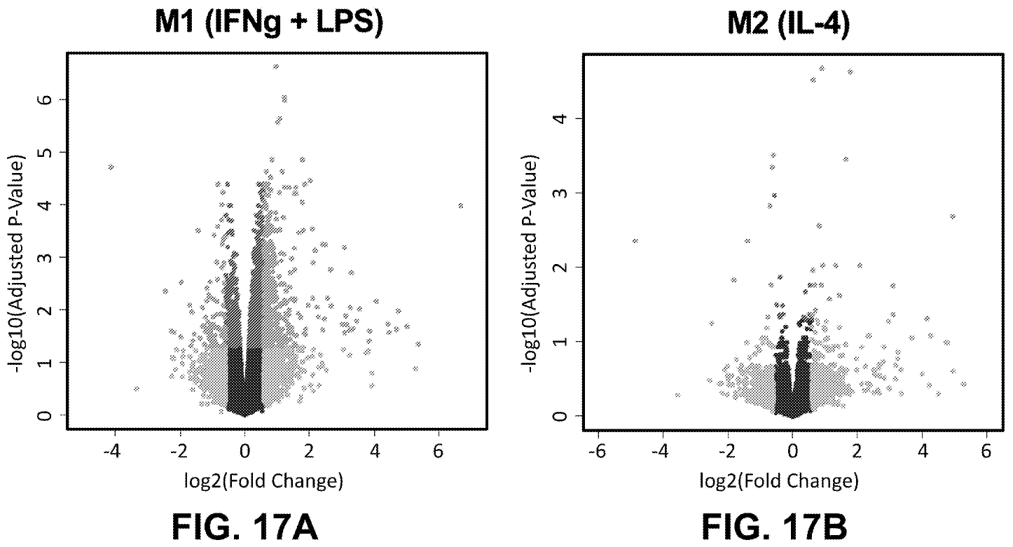

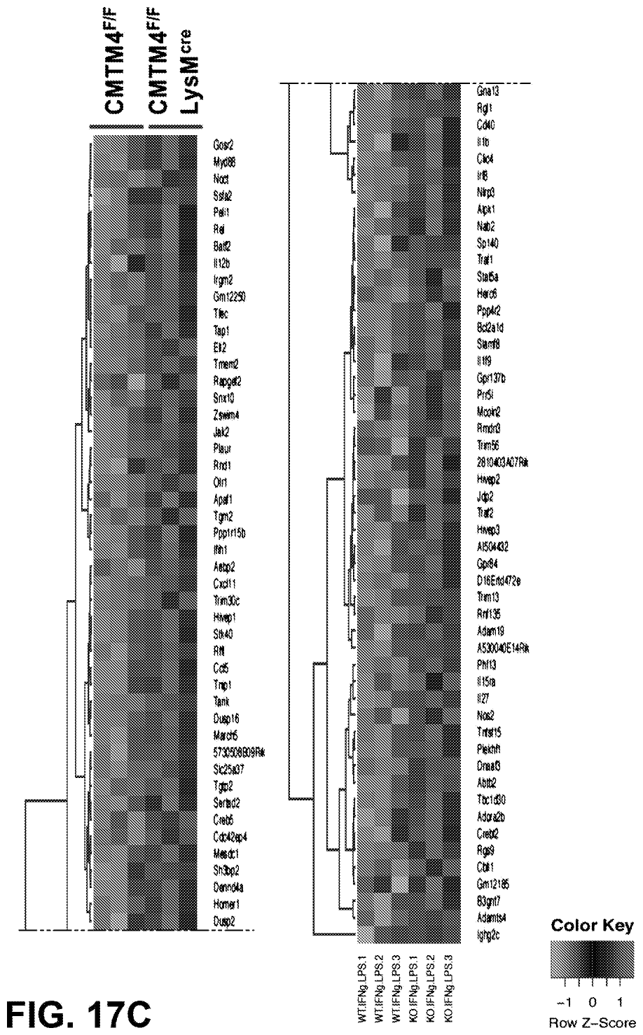

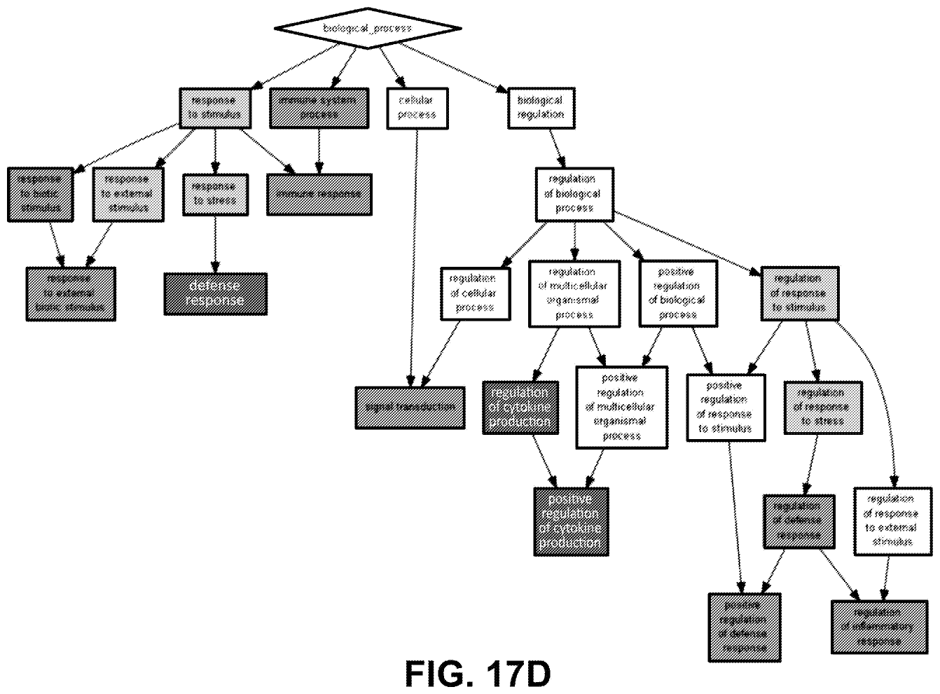

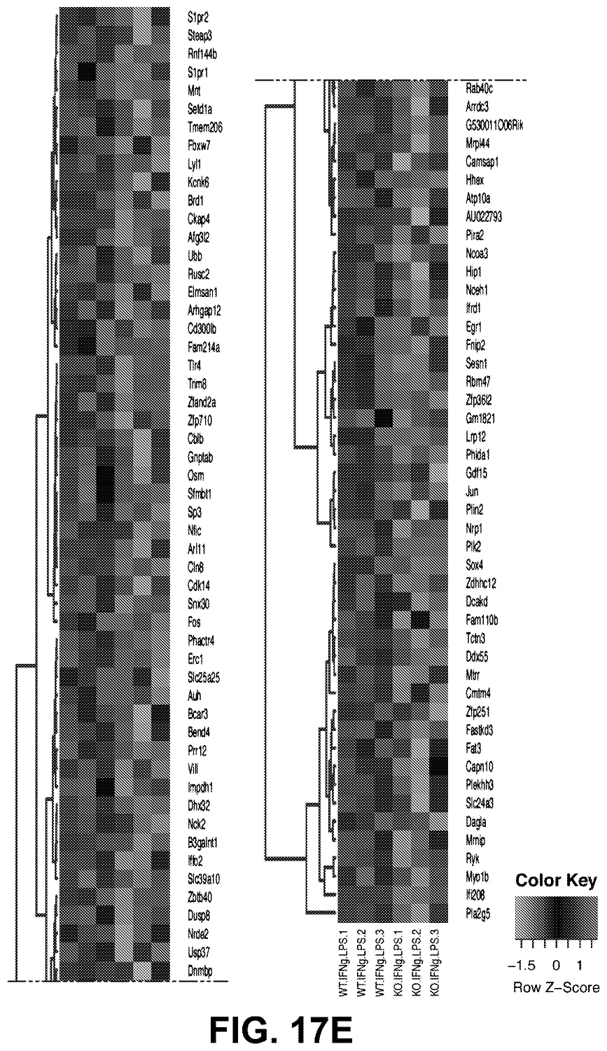

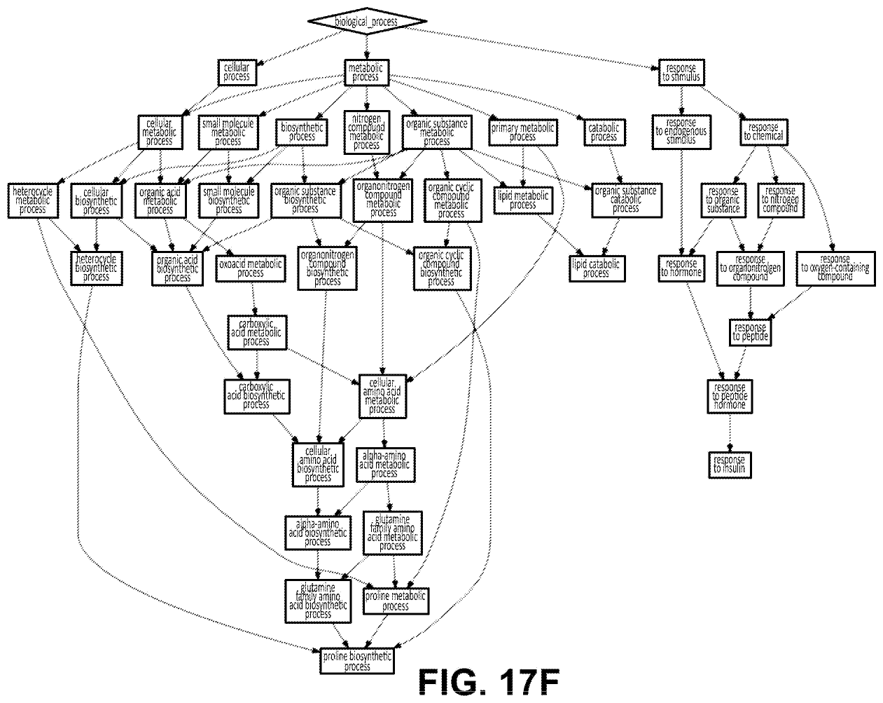

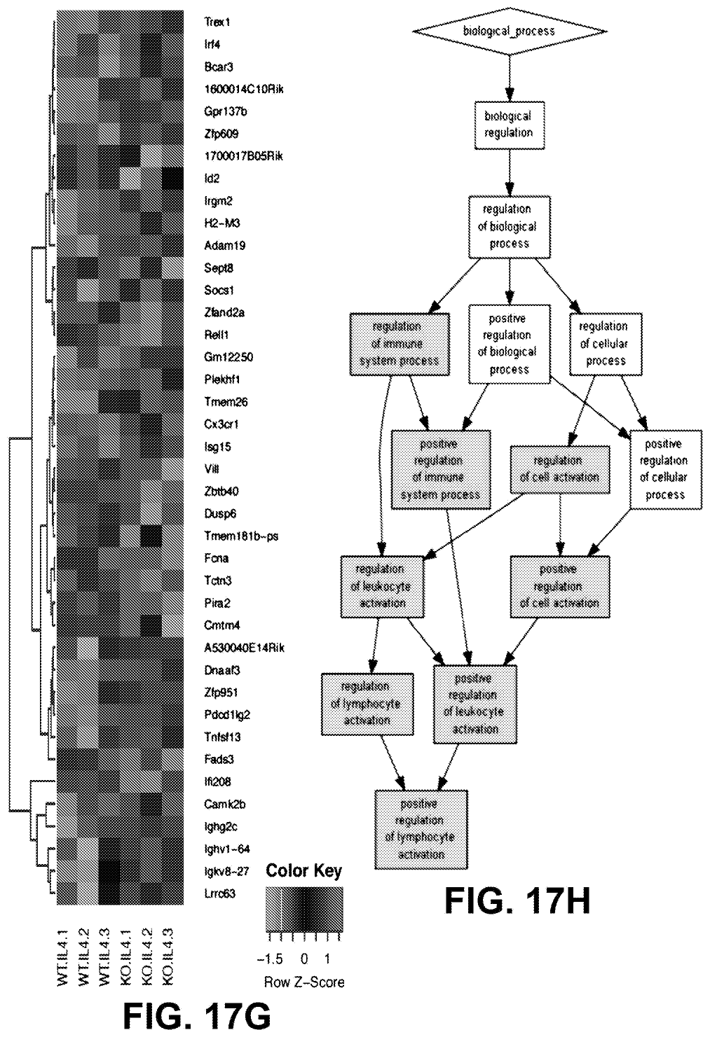

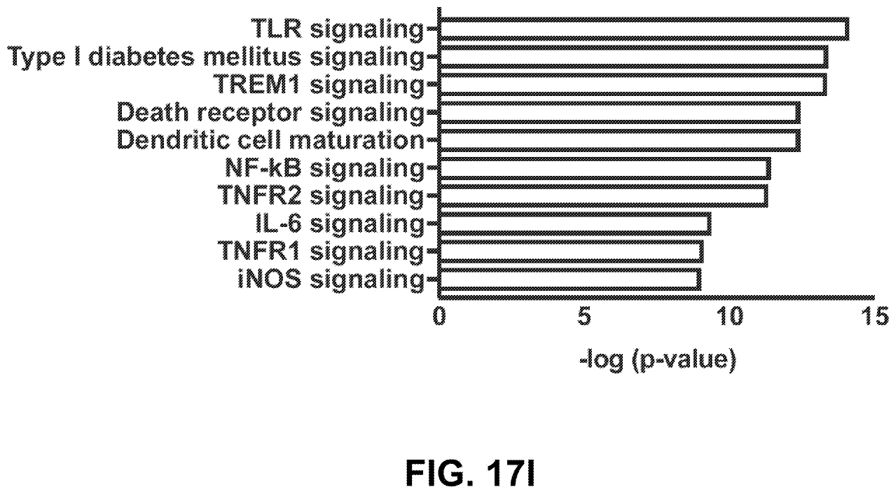

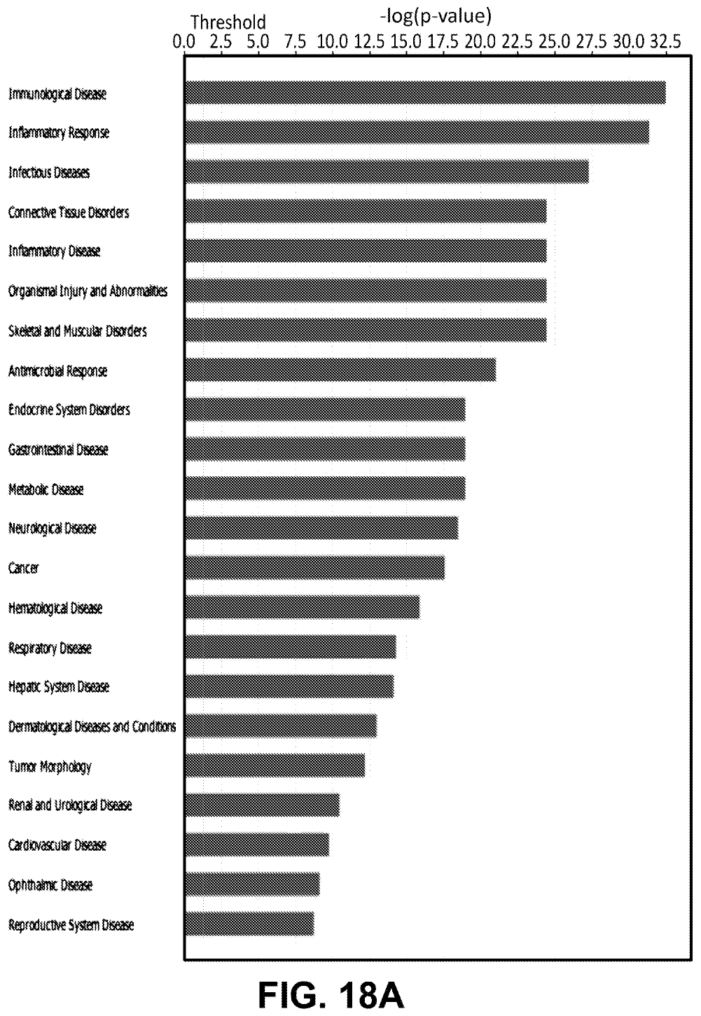

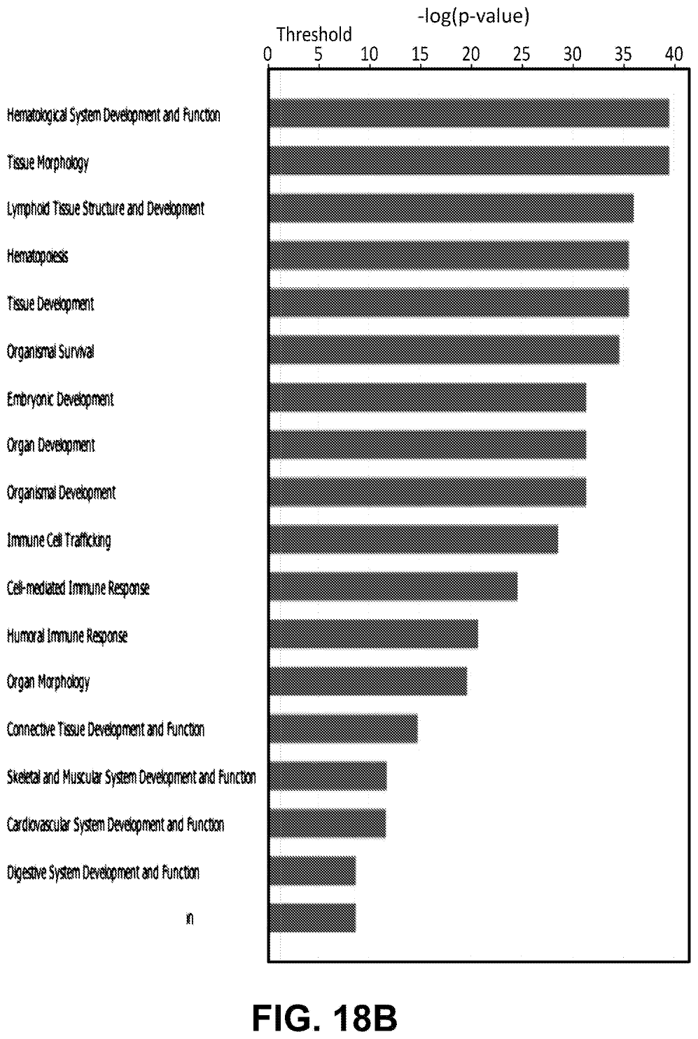

To gain additional insight into mechanisms by which CMTM4 regulates macrophage function, we performed RNAseq analysis with WT (CMTM4F/F) and CMTM4 KO (CMTM4F/FLysMcre) myeloid cells in M1 (IFNγ+ LPS) vs. M2 (IL-4) cultured conditions. CMTM4 KO resulted in substantially more transcriptional changes under conditions of M1 maturation compared to M2 maturation (FIGS. 17a and 17b). Among the 1,517 differentially expressed genes (DEGs), we divided the top 100 upregulated DEG and top 100 downregulated DEG in CMTM4 KO macrophages under M1 condition (FIGS. 17c and 17e) and applied gene ontology analysis using the Gorilla platform to provide a functional interpretation of the data. Top 100 upregulated DEG in CMTM4 KO macrophages indicated prominent changes in defense response and cytokine production (FIG. 17d). Top 100 downregulated DEG in CMTM4 KO macrophages under M1 condition showed significant changes in proline biosynthesis process, responses to insulin and hormone, and lipid catabolic process indicating important roles of CMTM4 in macrophage activation and metabolism (FIG. 17f). We also confirmed that DEG regulated by CMTM4 KO under M2 condition indicates CMTM4 KO has positive regulation of leukocyte activation (FIGS. 17g and 17h). Ingenuity Pathway Analysis (IPA) also identified statistically significant regulation of signaling on TLR, TREM1, NF-κB, TNFR, IL-6, and iNOS in CMTM4 KO macrophages indicating enhanced M1 activation (FIG. 17I). Furthermore, expected disease and disorders (FIG. 18a) and physiological system development and function changes (FIG. 8b) by CMTM4 KO in macrophages analyzed by IPA indicated that CMTM4 can have important roles in the regulation of inflammation and hematopoietic maintenance. Collectively, our profiling data indicated that CMTM4 plays an important role to control macrophage function and activation and is important in hematopoietic homeostasis.

(4) Myeloid CMTM4 KO Mice are More Susceptible to DSS-Induced Colitis and have Increased M1 Functional Phenotype

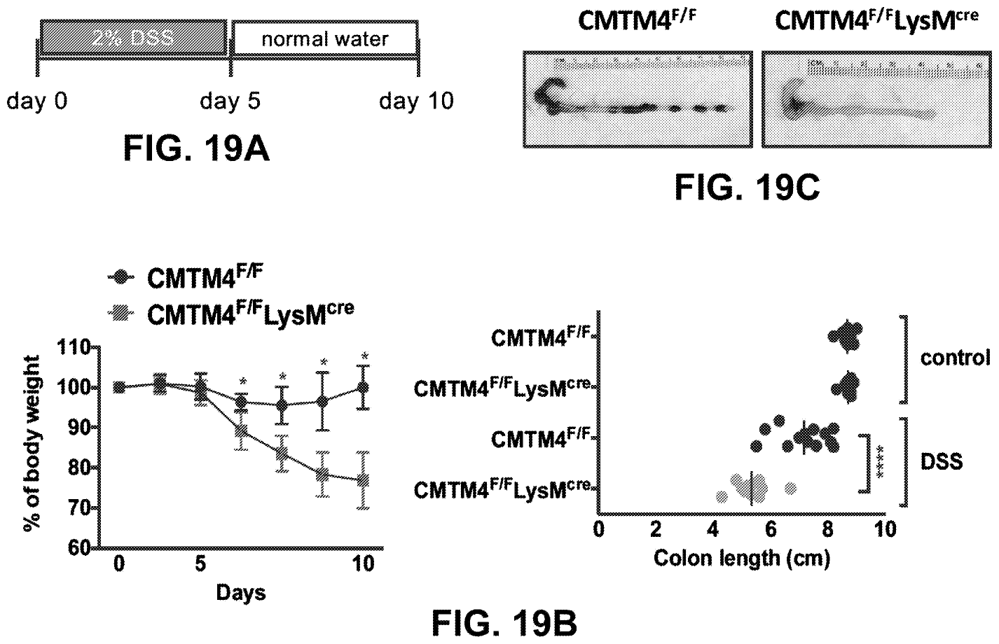

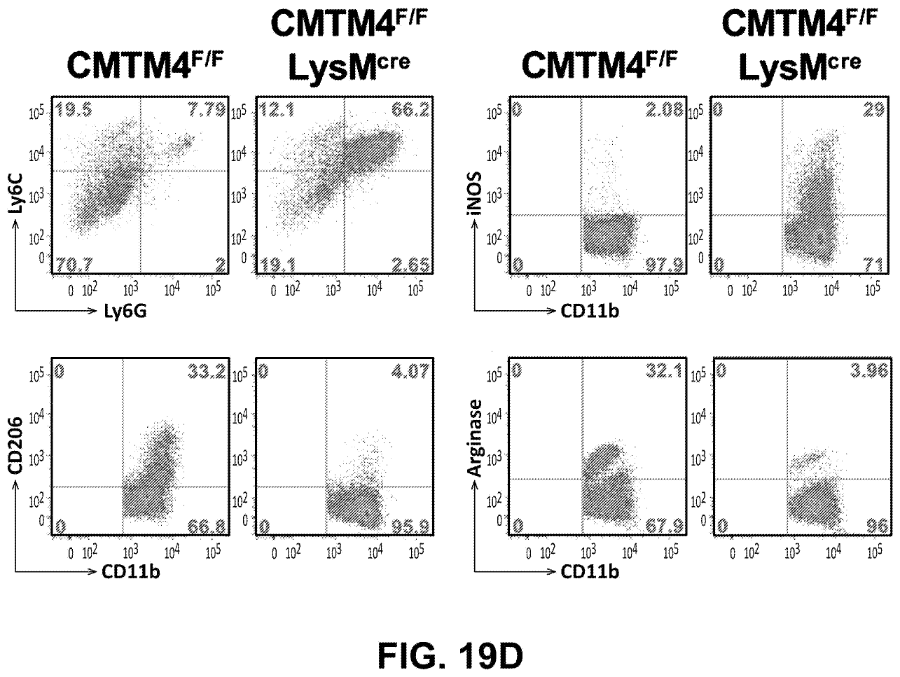

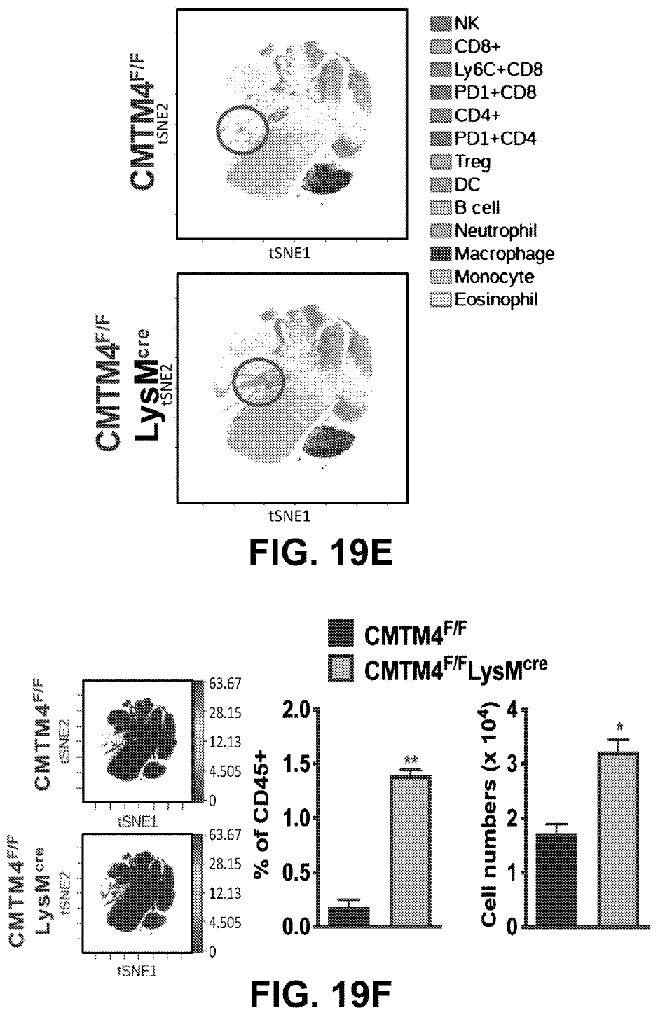

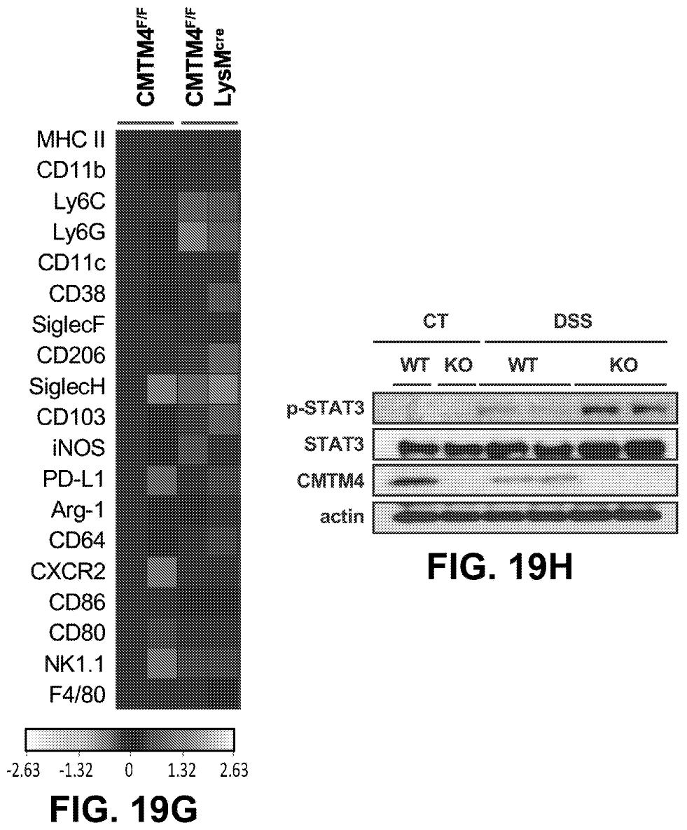

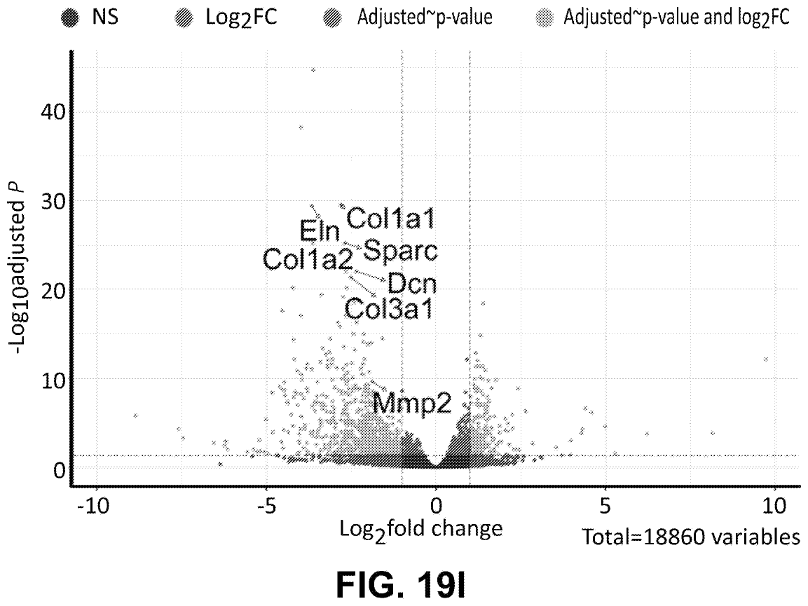

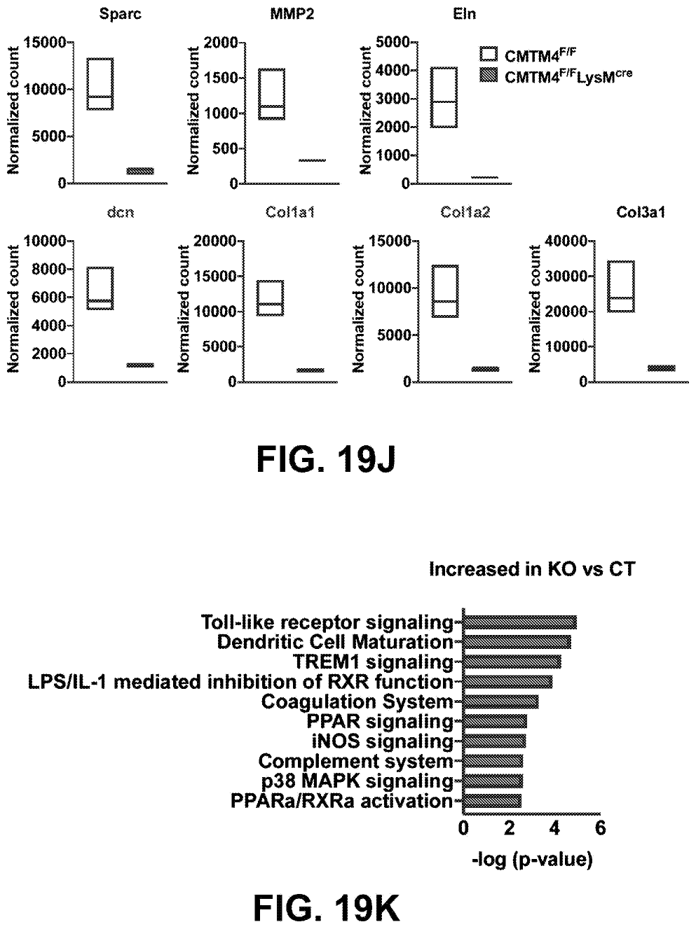

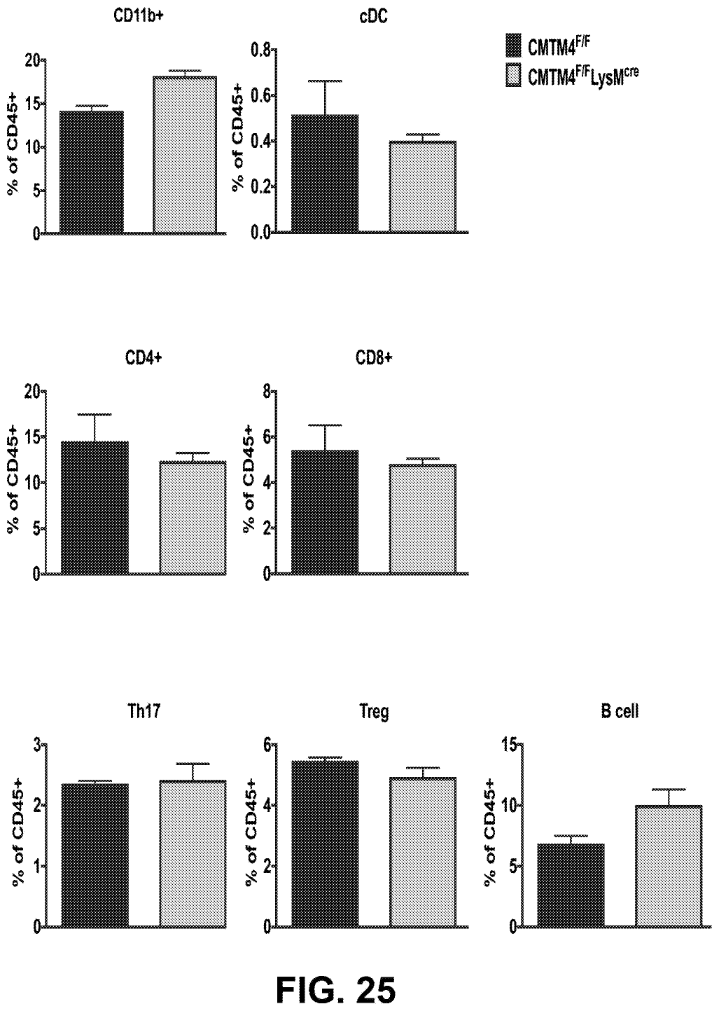

To evaluate whether the altered macrophage phenotype and function by CMTM4 influence intestinal homeostasis in vivo, WT and myeloid CMTM4 KO mice were treated with 2% dextran Sulfate sodium (DSS) (FIG. 19a). Myeloid CMTM4 KO mice with DSS treatment had significantly reduced body weight and shorter colon length than WT or littermate flox control mice indicating that myeloid CMTM4 KO probably had enhanced inflammation and intestinal damage (FIGS. 19b and 19c). To confirm whether myeloid CMTM4 KO mice have altered macrophage phenotype in lamina propria, we isolated and characterized immune cells in the lamina propria after DSS treatment. Flow cytometry analyses showed increased CD11b+Ly6G+Ly6C-neutrophils in lamina propria of DSS-treated myeloid CMTM4 KO mice. We also found that the myeloid cells in lamina propria from DSS-treated myeloid CMTM4 KO mice had increased iNOS expression and reduced expression of CD206 and arginase (FIG. 19d). Consistent with promoted M1 polarization in CMTM4 KO macrophages in vitro, myeloid CMTM4 KO mice also had increased M1 functional phenotypes in lamina propria and enhanced inflammation to DSS-induced colitis. Cytometry by time-of-flight (CyTOF) analysis confirmed increased immune cells in lamina propria from myeloid CMTM4 KO mice. Especially, the percentage and number of neutrophils were significantly increased in myeloid CMTM4 KO mice, but not control CMTM4 Flox/Flox mice in DSS treated mice (FIGS. 19e and 19f). However, CD11b+ myeloid cells, cDC, T cells, and B cells (FIG. 25) were not influenced by CMTM4 KO in DSS-induced colitis models. CyTOF analysis confirmed decreased CD206, siglec H, PD-L1 expression and increased iNOS expression in myeloid cells from CMTM4 KO mice. Furthermore, the expression of CD64 was increased but CXCR2 expression was decreased in myeloid cells from CMTM4 KO mice indicating M1-like functional phenotypes in CMTM4 KO mice (FIG. 19g).

### (5) Myeloid CMTM4 KO Increases Activation of STAT3 in Colon Tissue

Next, we determined whether myeloid CMTM4 can regulate signaling pathways involved in intestinal inflammation and colorectal cancer (CRC) development. STAT3 is linked to both stem cell reprogramming and renewal and constitutively, activation of STAT3 is associated with IBD. Furthermore, inflammatory cytokines activate STAT3 in premalignant cells to induce genes that stimulate cell proliferation, survival, growth, as well as angiogenesis, invasiveness, motility, and cytokine production. Interestingly, DSS treatment reduced CMTM4 expression in colon tissues. STAT3 activation was enhanced in colon tissues from myeloid CMTM4 KO mice (FIG. 19h). These data indicate that myeloid CMTM4 KO mice are more susceptible to inflammation and may promote colorectal cancer development under the inflammation condition.

### (6) CMTM4 KO Myeloid Cells Imbalance Extracellular Matrix (ECM) and Increase Inflammatory Signaling in Lamina Propria

To profile the transcriptome of intestinal immunity altered by CMTM4 KO in myeloid cells, we performed an RNAseq array with CD11b+ myeloid cells from inflamed lamina propria. The gene expression profile was calculated by the differential expression analysis and genes showing statistically significant changes in the expression level by adjusted p-value <0.05 were considered. Of 18,952 profiled transcripts, 1,681 were significantly altered by CMTM4 KO in myeloid cells from inflamed lamina propria (FIG. 19i). The most significantly changed genes were Sparc and ECM-related genes MMP2, Eln, Dcn, Col1a1, Col1a2, and Col3a1 (FIG. 19j). Sparc has been known to regulate the suppressive function of MDSC as well as to be involved in ECM remodeling. Furthermore, IPA indicated increased inflammatory signaling activation including TLR, TREM1, iNOS, and p38 signaling (FIG. 19k). The data indicate that CMTM4 KO myeloid cells can trigger tissue damage by change ECM and alteration of intestinal architecture due to chronic inflammation.

### (7) Myeloid CMTM4 KO Increases Inflammation-Induced Colitis-Associated Colorectal Cancer (CAC) Development

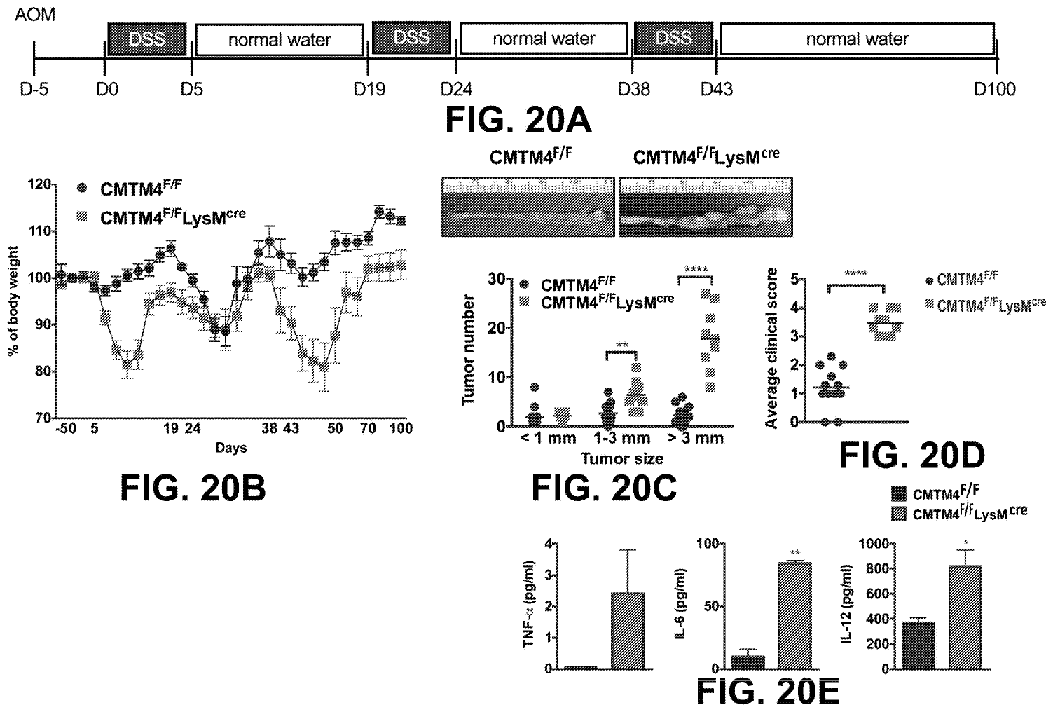

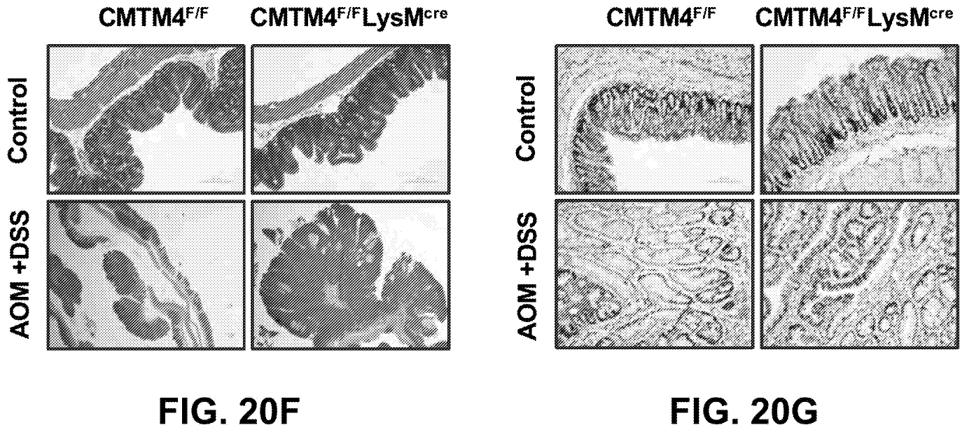

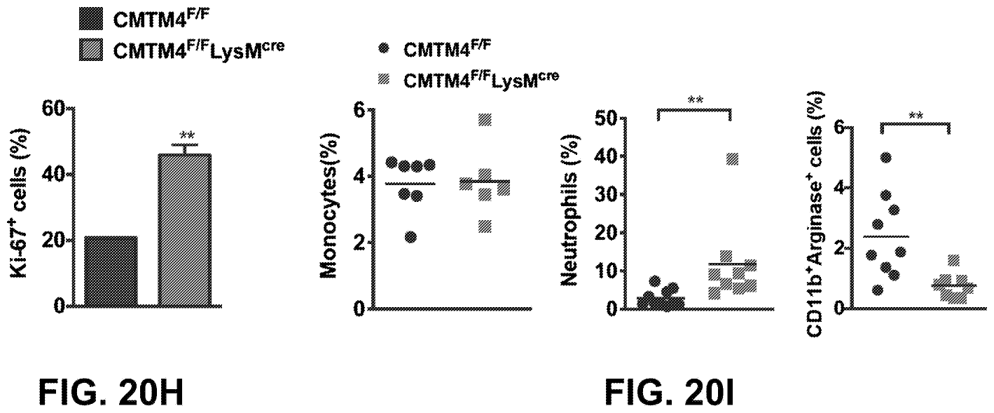

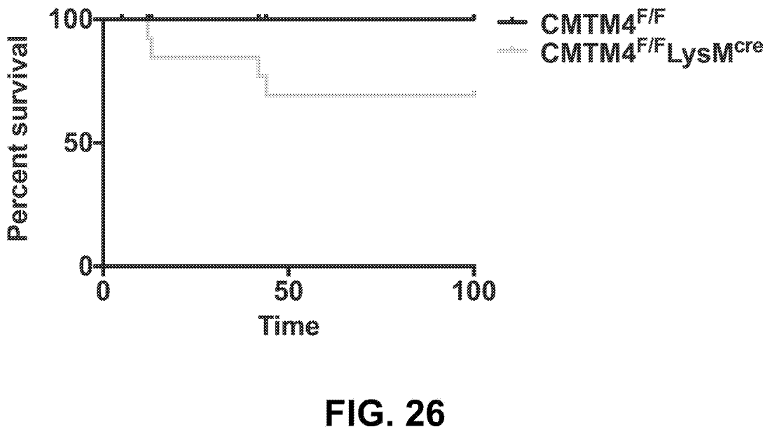

Since inflammatory bowel disease (IBD) is a major risk factor for CAC, we wondered whether altered macrophage function by CMTM4 influences CAC development and progression. To investigate the role of myeloid CMTM4 in CAC, we used a protocol to combine the carcinogen azoxymethane (AOM) with DSS-induced colitis (FIG. 20a). During the treatment of the AOM/DSS, bodyweight loss has observed; however, it recovered (FIG. 20b) and the average clinical score was higher (FIG. 20d) in myeloid CMTM4 KO mice compared to control mice consistent with the colitis model. While all tested control mice survived, 30% of myeloid CMTM4 KO mice died during the treatment of the AOM/DSS (FIG. 26). After treatment of the AOM/DSS, myeloid CMTM4 KO mice and control littermates developed colon tumors mainly in the distal to the middle colon, which is the predominant localization of human colorectal tumors (FIG. 20c). We noticed that myeloid CMTM4 KO mice had more macroscopic tumors and the tumor load and sizes were higher than WT mice (FIG. 20c). Interestingly, the level of circulating inflammatory mediators such as TNF-α, IL-6, and IL-12 were upregulated in myeloid CMTM4 KO mice at the end of the AOM/DSS protocol (FIG. 20e). This is consistent with increased inflammatory mediator expression and production in CMTM4 KO macrophages. Histological analyses confirmed higher-grade tumors in myeloid CMTM4 KO mice (FIG. 20f). To evaluate whether the increased inflammation by CMTM4 KO influences cell proliferation in the colon, we directly analyzed cell proliferation in colon tissue sections by Ki-67 staining. Consistent with the profound effect on tumor size and number, we found a significant higher number of Ki-67+ proliferative cells compared with control tissues indicating that myeloid CMTM4 under the inflammation condition promotes epithelial cell proliferation (FIGS. 20g and 20h).

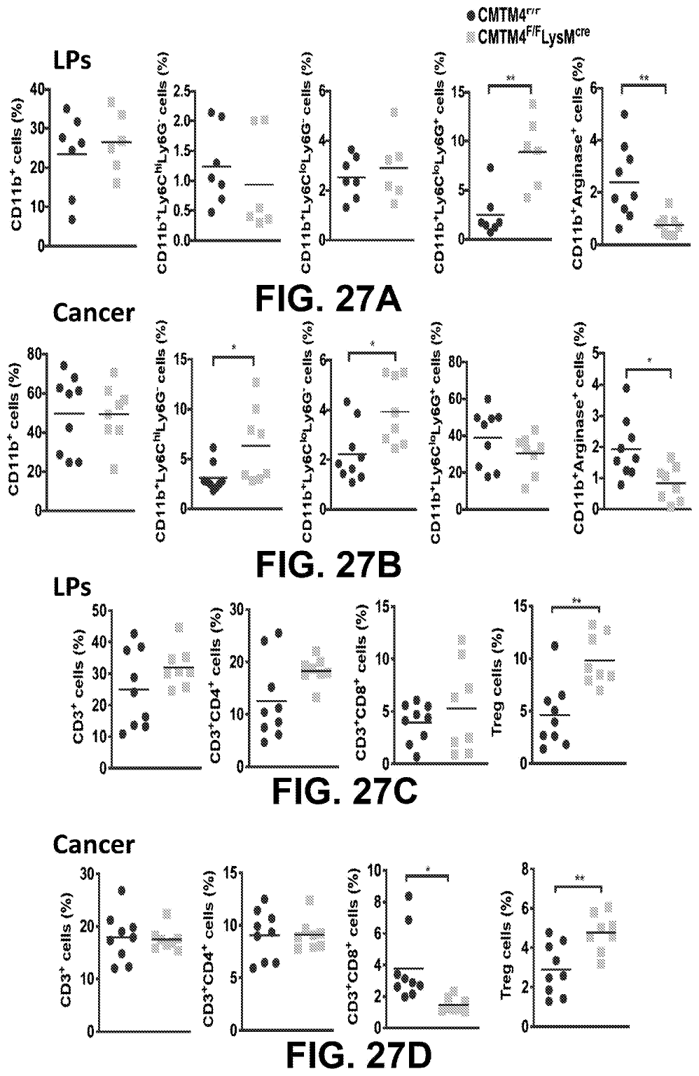

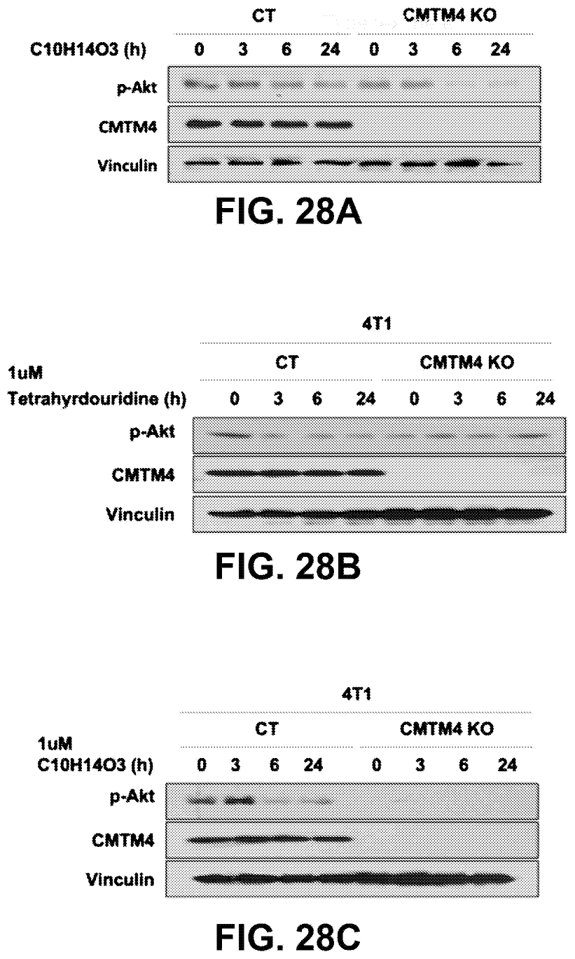

To evaluate whether myeloid CMTM4 controls immune cell infiltration and function during CAC development, we characterized myeloid cells and T cells in lamina propria and colorectal tumor tissues in mouse CAC models. We found that neutrophils were significantly elevated whereas monocyte infiltration was not changed in lamina propria from myeloid CMTM4 KO mice (FIG. 20i). Surprisingly, arginase+M2-like functional macrophages were diminished in lamina propria and tumor from myeloid CMTM4 KO mice (FIG. 20i and FIG. 27b). We found that Treg cells from lamina propria and tumor were dramatically increased whereas tumor-infiltrating CD8 T cells were decreased in myeloid CMTM4 KO mice (FIGS. 27c and 27d).

### (8) Myeloid CMTM4 KO Inhibits Tumor Development on Intestinal or Mammary Tumor Transgenic Mice and Transplant Tumor Models

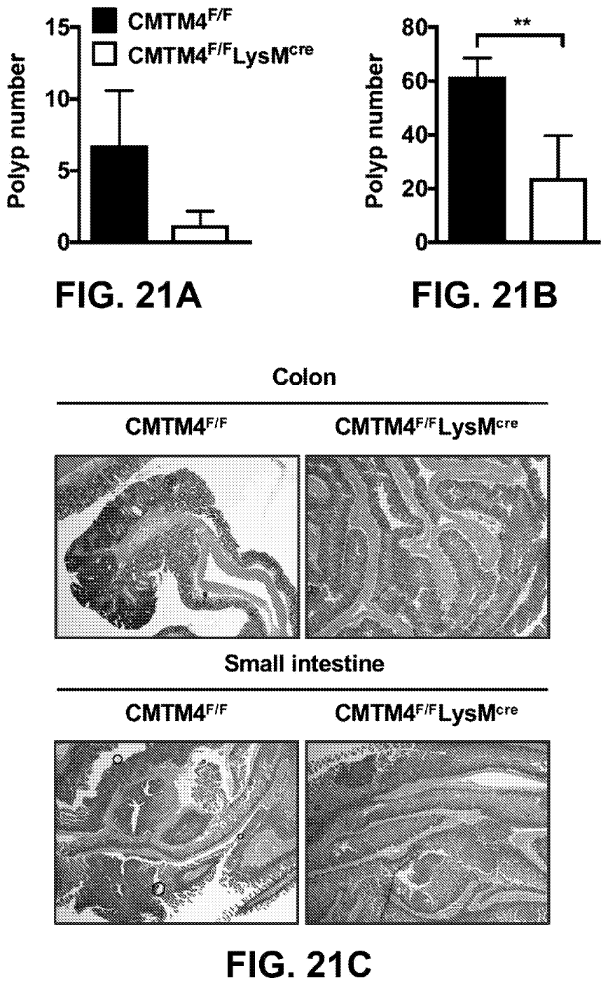

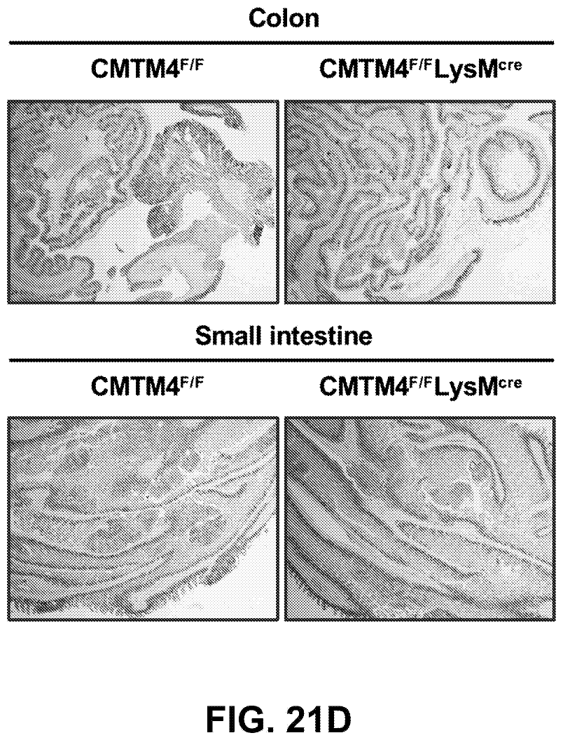

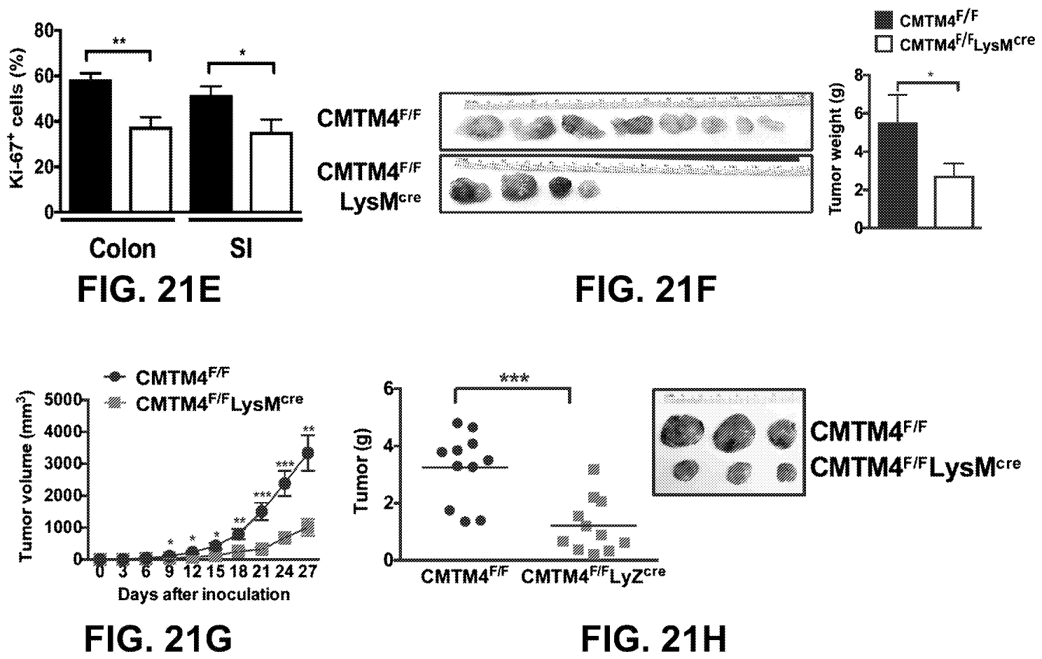

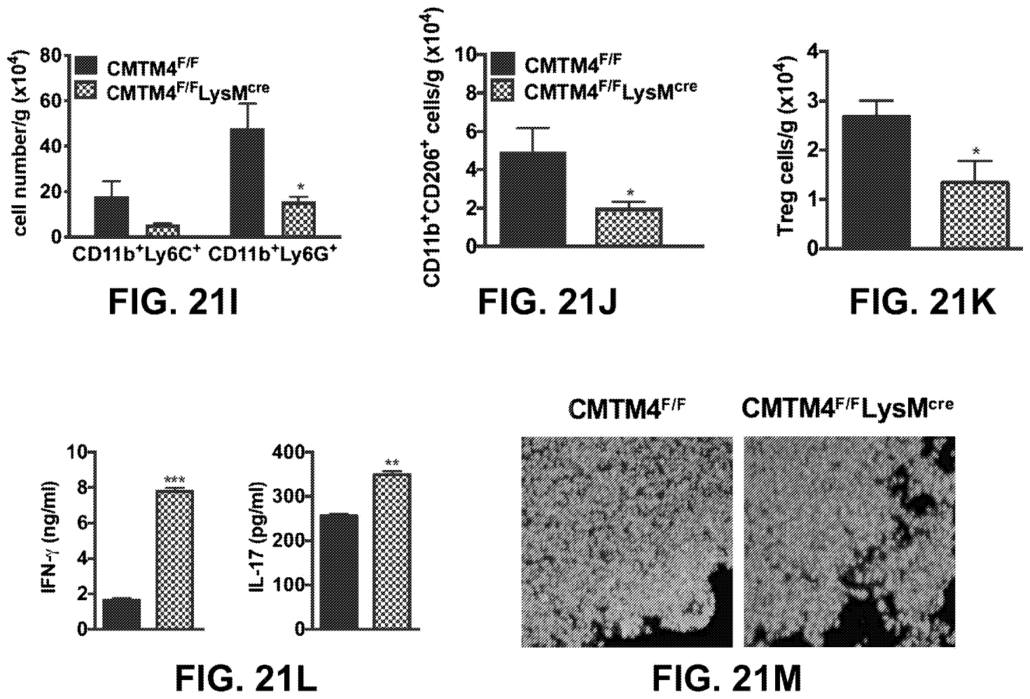

Next, we wondered whether the altered macrophage function by CMTM4 KO has the same effects in genetically engineered tumor models. To address this, we used a genetically modified colorectal cancer mouse model. APCmin/+ mice carry a truncational mutation of the Apc gene and spontaneously develop tumors in the small intestine and less frequently in the colon. To see the effect of myeloid CMTM4 on genetically induced intestinal tumor development, the bone marrow cells from control and myeloid CMTM4 KO mice were transferred to APCmin/+ mice. After about 120 days, the mice developed tumors predominantly in the small intestine and less in the colon. The APCmin/+ mice transferred myeloid CMTM4 KO bone marrow cells showed reduced tumor formation in the small intestine and the colon (FIGS. 21a and 21b). Histological analyses confirmed lower-grade tumors in myeloid CMTM4 KO bone marrow transferred APCmin/+ mice (FIG. 21c). Ki-67 staining indicated diminished Ki-67+ proliferating cells in both the small intestine and colon from APCmin/+ mice transferred myeloid CMTM4 KO bone marrow cells (FIGS. 21d and e). We have further evaluated other types of genetically engineered tumor models. We confirmed the effects of myeloid CMTM4 KO in MMTV-PyMT transgenic mice that develop multifocal mammary tumors. MMTV-PyMT mice transferred with control bone marrow cells developed about 8 to 10 tumor nodule burdens in a whole-body whereas the MMTV-PyMT mice transferred with myeloid CMTM4 KO bone marrow cells showed reduced total tumor weight and tumor formation having only 3 to 4 tumor burdens (FIG. 21f).

As the most common approach for evaluation of antitumor response is using the traditional transplanted mouse tumors, we have examined the contribution of CMTM4 in macrophages to tumor growth in the transplant model. LLC cells were transplanted subcutaneously into the mice and the tumor size was evaluated every 3 days. Interestingly, tumor growth (FIG. 21g) and tumor weight (FIG. 21h) was significantly reduced in myeloid CMTM4 KO mice when compared to control mice. Interestingly, tumor-infiltrating neutrophils were significantly diminished in myeloid CMTM4 KO mice (FIG. 21i). Furthermore, M2-like CD206+CD11bb+ cells were diminished in tumor tissues from myeloid CMTM4 KO mice (FIG. 21j). To identify the effect of myeloid-specific CMTM4 deficiency on T cells in the tumor microenvironment, we evaluated T cells in the spleen and tumor from tumor-bearing CMTM4F/F and CMTM4F/FLysMcre mice. Interestingly, the numbers of Treg cells were significantly reduced in the tumor from tumor-bearing myeloid CMTM4 KO mice when compared to control tumor-bearing mice (FIG. 21k). When purified T cells were stimulated with anti-CD3/anti-CD28, the production of IFN-γ and IL-17 was significantly enhanced in T cells isolated from tumor-bearing myeloid CMTM4 KO mice compared to those from tumor-bearing control mice (FIG. 21I). Moreover, reduced angiogenesis in the tumor from myeloid CMTM4 KO mice was confirmed with immunofluorescent staining for CD31 (FIG. 21m). Therefore, these data indicate that M1 functional myeloid cells by CMTM4 KO can prevent genetically modified spontaneous tumor development and transplanted tumor progression whereas promoting inflammation-induced cancer development.

(9) Single-Cell RNAseq Analysis of Inflamed Lamina Propria Cells Vs. Transplanted Tumor-Infiltrating Cells

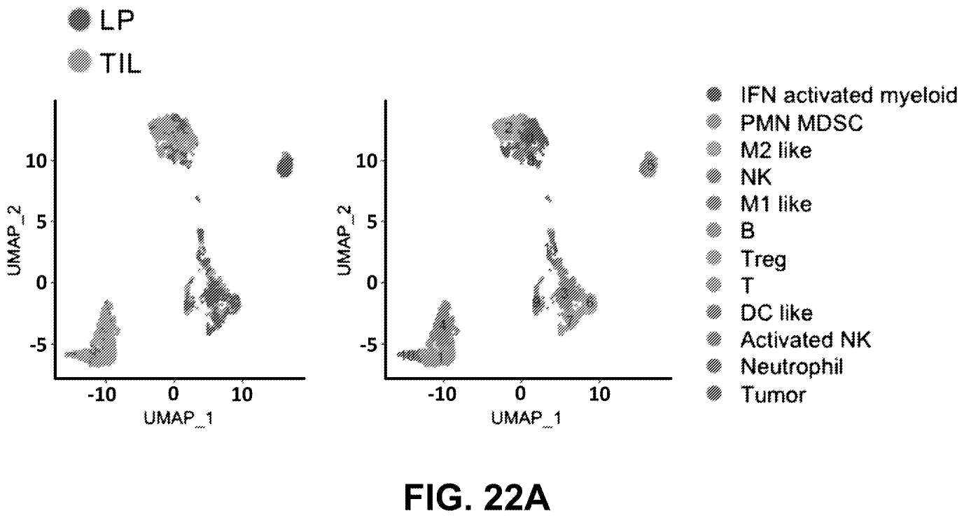

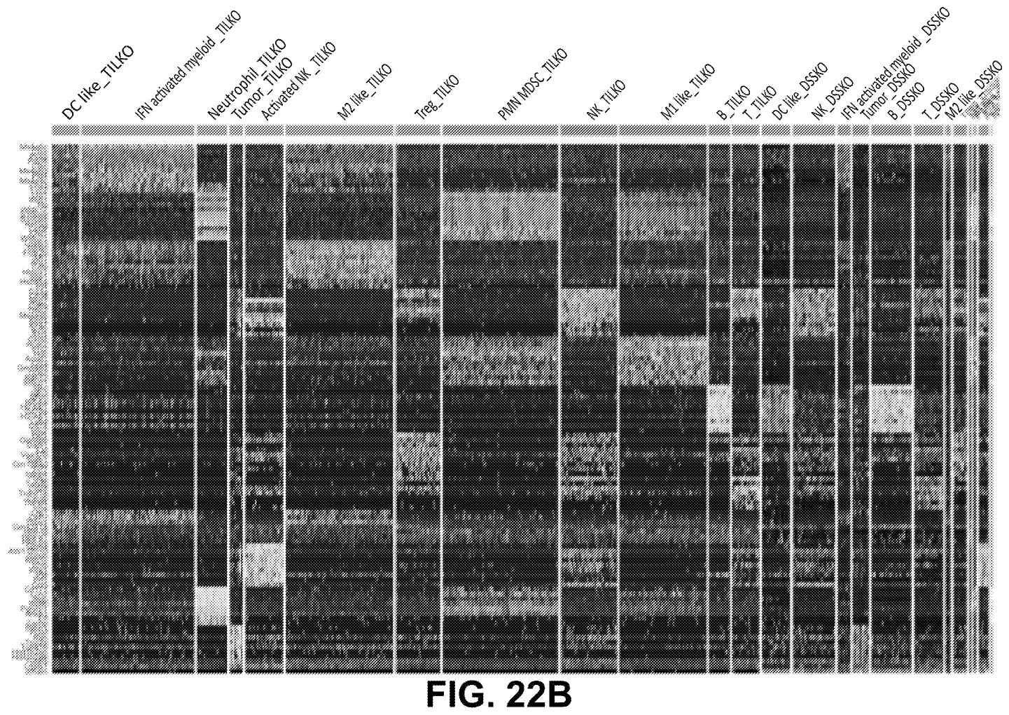

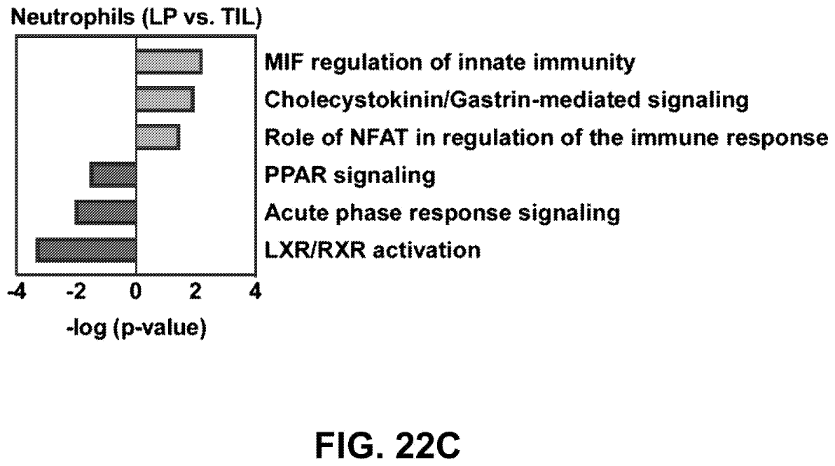

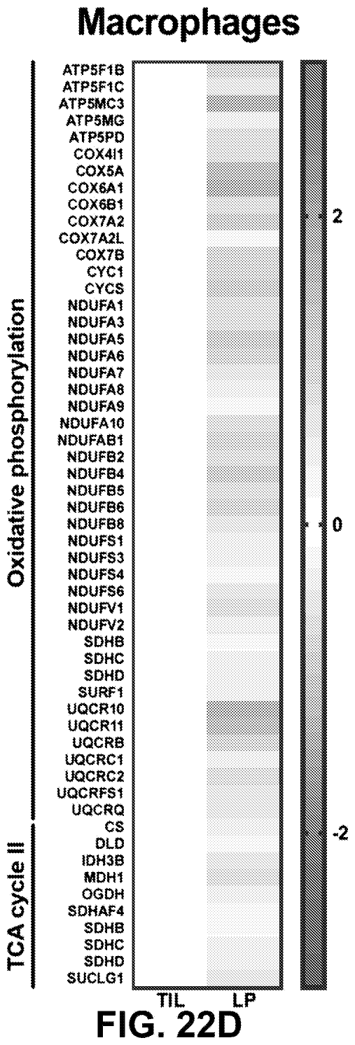

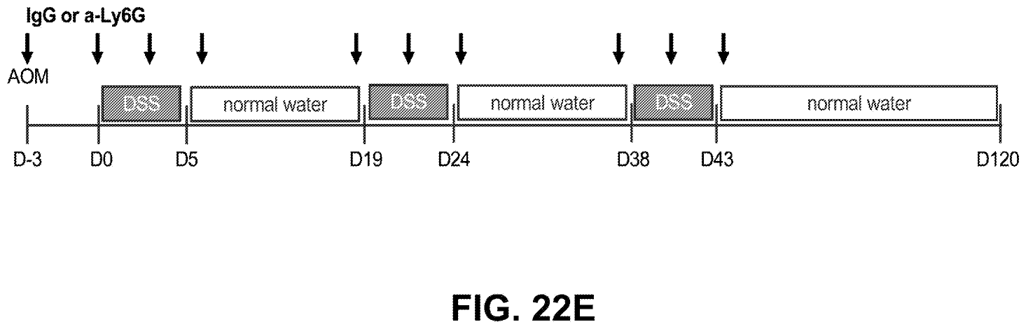

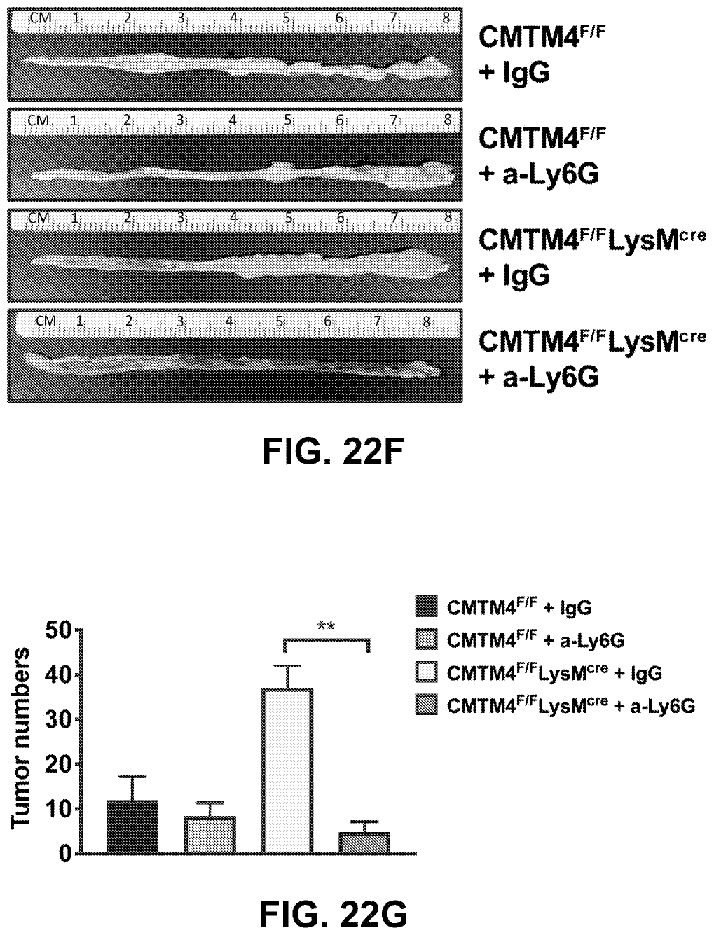

The results presented in our results from different tumor models indicated that M1 macrophages have opposite functions on different types of tumor microenvironment. This prompted us to compare the macrophages between inflammatory conditions vs. the transplanted tumor. To dissect the cells in different environments of tumor developments, scRNAseq was performed on isolated immune cells from DSS-treated lamina propria and transplanted tumors of control and myeloid CMTM4 KO mice. Cell clustering allowed the identification of 12 clusters including IFN activated myeloid cells, PMN-MDSC, M2-like myeloid cells, NK cells, M1-like myeloid cells, B cells, Treg cells, T cells, DCs, activated NK cells, neutrophils, and tumor cells. Each cluster was characterized by a specific gene signature (FIG. 22b). Since a major difference between inflammation-induced CRC vs. genetically modified CRC or transplanted tumor models in myeloid CMTM4 KO mice was neutrophil infiltration, we first compared gene expression in neutrophils between lamina propria and transplanted tumor by IPA analysis. Interestingly, LXR/RXR signaling, which suppresses metastasis in the cancer microenvironment, was most significantly reduced and MIF had significantly upregulated, which has been reported to promote the M2 alternative macrophage in neutrophils from lamina propria compared to transplanted tumor-infiltrating neutrophils in myeloid CMTM4 KO mice (FIG. 22c). To see whether suppressive immune phenotypes from neutrophils in lamina propria altered macrophage functions, we performed IPA analysis. Expression of genes that are enhanced in M2 macrophages such as oxidative phosphorylation and TCA cycle 11 increased in lamina propria myeloid CMTM4 KO cells compared to tumor-infiltrated leukocytes from myeloid CMTM4 KO mice (FIG. 22d). Taken together, these data indicate that neutrophils can modulate M1 macrophages functional phenotype toward to have M2-dependent phenotypes especially in the metabolic pathways and oxidative stress in an inflammatory condition that promotes tumor development and progression.

### (10) Depletion of Neutrophils Controls the Development of CRC in Myeloid CMTM4 KO Mice

Next, we evaluated whether neutrophils play an important role in inflammation-induced CRC development. Control or neutrophil depleting antibodies were injected during DSS treatment in AOM/DSS-induced CRC models (FIG. 22e). After 120 days, neutrophil-depleted mice showed significant reduction on CRC development in myeloid specific CMTM4 KO mice, but do not have significant difference in control mice (FIGS. 22f and 22g). Thus, these results indicate that inflammatory CMTM4 KO macrophages control tumor development and progression by communicating with neutrophils. Neutrophils might reprogram macrophages to play different roles under the inflammatory condition.

(11) Neutrophils Reprogram of M1 Macrophages into Tumor-Promoting Macrophage Under Inflammatory Condition

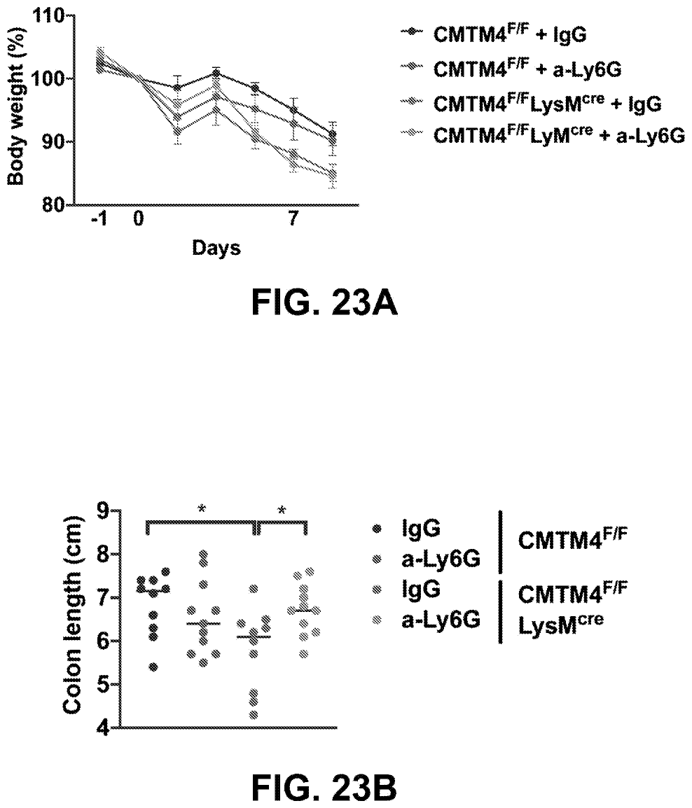

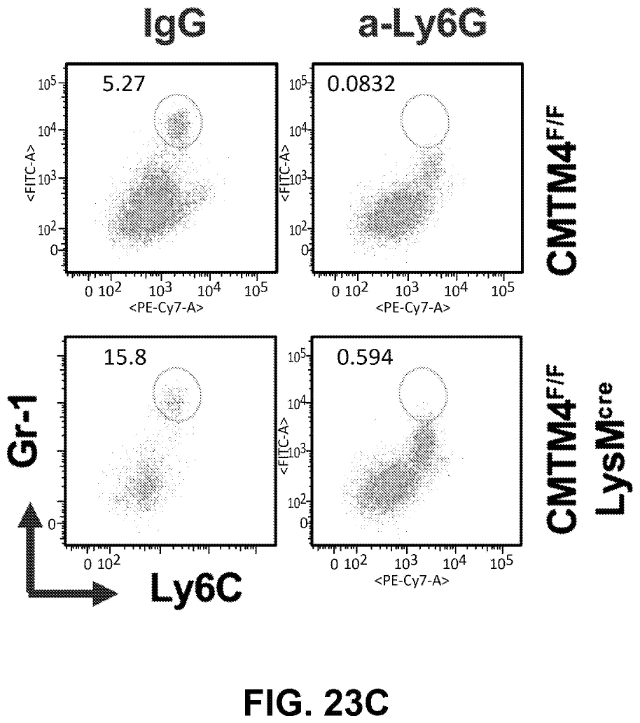

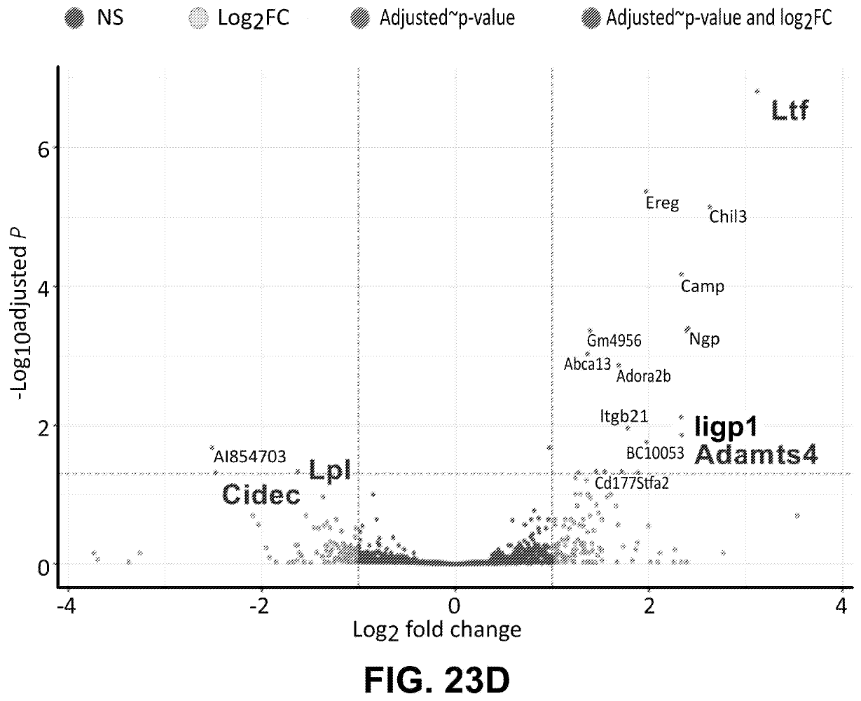

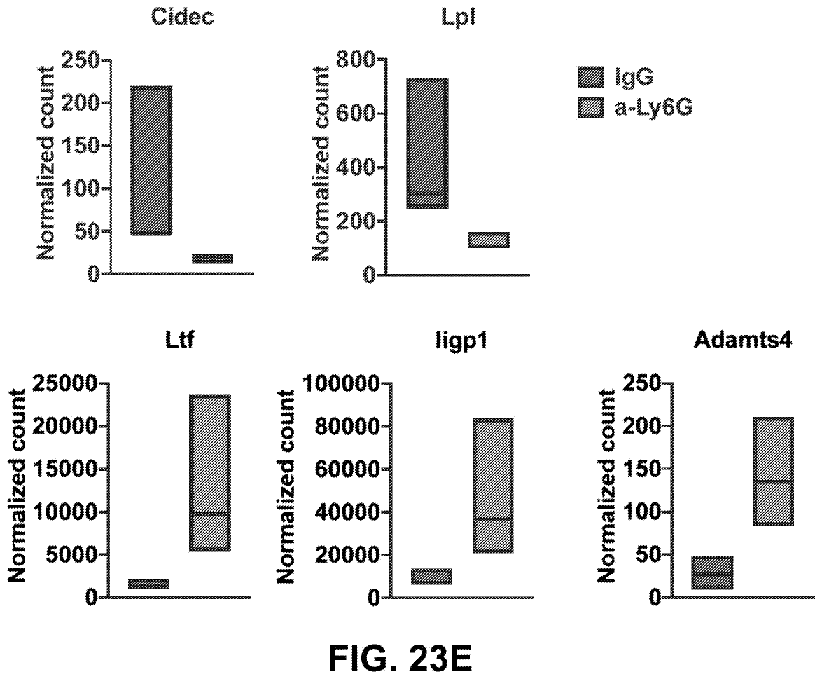

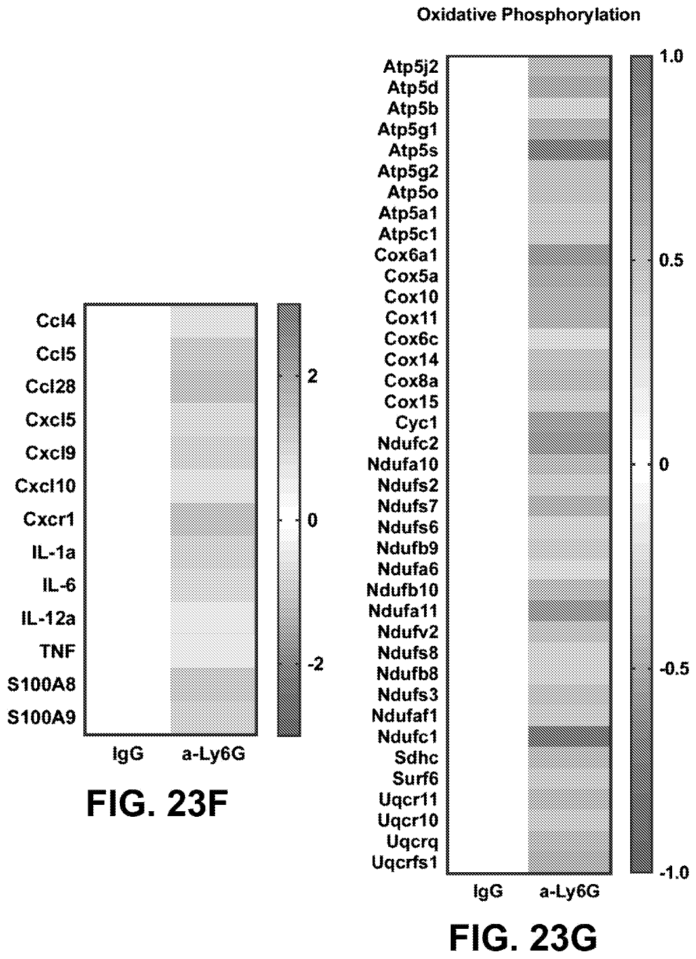

To determine whether neutrophils influence macrophage phenotypes or functions in inflammatory conditions, neutrophils were depleted with anti-Ly6G neutralizing antibodies in DSS-induced IBD models. Although body weights were not significantly changed by neutrophil depletion in both control and myeloid CMTM4 KO mice (FIG. 23a), DSS-mediated reduced colon length was recovered by neutrophil depletion in myeloid CMTM4 KO mice but not in control mice (FIG. 23b). Successful depletion of neutrophils was confirmed (FIG. 23C). Next, we isolated myeloid cells from lamina propria and performed RNAseq array to gain insight into mechanisms by which neutrophils control CMTM4 KO macrophage under inflammatory conditions. The gene expression profile was calculated by the differential expression analysis and genes showing statistically significant changes in the expression level by adjusted p-value <0.05 were considered. Most significantly decreased genes by neutrophil depletion included cell-death-inducing DFF45-like effector C (Cidec), which is a PPARγ target gene and suppresses TNF-α-induced lipolysis, and lipoprotein lipase (Lpl) which is a fatty acid uptake gene and is induced by PPARγ-coactivator-1β activation. Most significant increased genes by neutrophil depletion included lactoferrin (Ltf), which switches M2 to M1 macrophages, Interferon-inducible GTPase 1 (Iigp1), and Adamts4, which are enhanced by inflammatory cytokines (FIG. 23e). We also found that expression of inflammatory chemokines including CCL4, CCL5, CXCL5, CXCL9, and CXCL10 and cytokines including IL-1α, IL-6, IL-12a, TNF, and S100A8/9 was increased in myeloid cells after neutrophil depletion (FIG. 23f). Furthermore, we found that genes regulating oxidative phosphorylation were increased in lamina propria macrophages (FIG. 22e); however, they were decreased after neutrophil depletion (FIG. 23g). These data indicate that neutrophils can reprogram M1 macrophages by interfering with their metabolic and inflammatory pathways and anti-tumor function for promoting tumor growth and immune suppression under the inflammation condition. Proper regulation of macrophage and neutrophils crosstalk thus can achieve M1 macrophage therapeutic efficacy even in the inflammatory tumor microenvironment.

### b) Discussion

CMTM4 is a novel inflammatory regulator and is known to be involved in the regulation of tumor progression. In this study, we unveil a critical role of CMTM4 in macrophages and differential roles of macrophages during cancer progression. Although it has been known that inflammation plays a critical role in tumorigenesis for decades, a direct causal relationship between inflammation and tumor is not yet proven. The tumor microenvironment contains innate immune cells including macrophages, NK cells, and dendritic cells. Macrophages are one of important players since they are most frequently found and abundant within the tumor microenvironment and can have tumor-promoting as well as antitumor functions2. While the roles of macrophages in genetically modified and transplanted tumors have not been compared with the same genetic macrophage polarization system, it is veiled to unequivocally assess the overall impact of macrophages and inflammation-mediated tumorigenesis since direct in vivo models for evaluating the relationship between macrophages and inflammatory tumor growth is missing.

Recent studies have shown the differential roles of tumor-regulating factors in distinct types of carcinogenesis. Loss of p53 in intestinal epithelial cells was not sufficient to initiate spontaneous intestinal tumorigenesis but enhanced carcinogen-induced tumor incidence, development to invasive cancer, and lymph node metastasis. Downregulation of p38a in intestinal epithelial cells increased cancer development whereas inhibition of p38a in tumor epithelial cells reduced tumor burdens. TGF-β signaling pathways showed tumor suppressor effects in normal cells and early carcinomas. However, these protective effects of TGF-β were lost as tumors developed and progressed showing switched effects to promote cancer progression, invasion, and tumor metastasis. Thus, these studies imply different functions of tumor-regulating genes in different types and stages of cancer. Our results indicate that macrophages also have a context-dependent function in the tumor microenvironment. In our system, macrophage-specific CMTM4 KO leading to colorectal cancer development indicates that M1 macrophages may serve as a potential risk factor in inflammation-mediated colorectal cancer development (FIG. 20). However, in genetically modified tumor models, CMTM4 KO M1 macrophages suppressed tumor progression by inhibiting proliferation of transformed epithelial cells indicating that M1 macrophages have anti-tumor functional roles during tumor progression (FIG. 21a-f). We also confirmed that myeloid CMTM4 KO mice had reduced tumor growth when cancer cells were inoculated through the transplant system (FIGS. 21g and h). Thus, our data indicate that macrophages can have different roles depending on tumor microenvironment. The regulation of macrophage polarization in the tumor microenvironment may reflect more efficacious novel therapies through the controlling of its metabolism pathways and change functions as seen in FIG. 23d-g.

Macrophages are pretty well characterized but still have the complexity to control its identity and functions due to diversity, the possibility of inter-conversion between macrophage states, and microenvironmental sensitivity. Especially, the function of macrophages in disease or other challenges may be controlled by stimulus-specific factors that respond to environmental signals. Thus, identification of the molecules associated with the diverse changes of macrophages is crucial for elucidating the molecular basis of disease progression and to develop new therapeutic targets. CMTM4 expression was increased during treatment of M-CSF which is involved in M2 polarization whereas it was decreased by treatment with GM-CSF or IFNγ/LPS inducing M1 polarization (FIG. 14a-14d). Furthermore, CMTM4 itself can control macrophage differentiation and function showing that CMTM4 KO macrophages had M1 functional phenotypes and activation of signaling involved in M1 differentiation (FIGS. 15A, 15B, 15C, 15J, 16A, and 16B). The alteration of macrophage function by CMTM4 KO was also confirmed by RNAseq data analysis. CMTM4 KO macrophages had significantly increased defense response, cytokine production, and regulation of inflammatory response that are functional features of M1 macrophages (FIG. 17d). Furthermore, CMTM4 KO macrophages indicated enhanced signaling on TLR, TREM1, NF-κB, TNFR, IL-6, and iNOS (FIG. 17I). TLR signaling is well known to be involved in M1 macrophage polarization. TLR activation recruits signaling adaptors MyD88 and TIR domain-containing adaptor-inducing interferon-β (TRIF). Then, MAPK and NF-κB are activated resulting in the production of inflammatory cytokines. TREM1 activates myeloid cells by associating with adaptor protein DAP12 and amplify inflammatory responses. Therefore, CMTM4 KO macrophages can have reinforced inflammatory responses through the initiation of TLR and amplification by TREM1.

Several studies have shown that macrophages are one of the important determinants of the efficacy of tumor therapies. The density of tumor-associated macrophages can be a predictive biomarker of responses to postsurgical adjuvant chemotherapy in pancreatic adenocarcinoma. Taxane docetaxel works through the depletion of M2-like macrophages and activation of M1-like macrophages. Mycobacterium bovis bacillus Calmette-Guerin (BCG), which is used for bladder cancer immunotherapy, induces macrophage-mediated cytotoxicity through inflammatory cytokine production. An agonistic antibody to the TNF receptor family member, CD40, skewed M1 macrophages and enhanced the efficacy of gemcitabine in pancreatic cancer. However most of these models are on the transplant or transgenic mice model which cannot account under the inflammation environment. Myeloid CMTM4 KO mice had increased Th1 and Th17 cells and reduced Treg cells in transplanted tumor models (FIG. 21k and 21l) whereas had increased Treg cells in the inflammation-mediated CRC models (FIG. 27). Thus, regulation of CMTM4 expression in macrophages can have therapeutic efficacy in cancer.

Neutrophils are hallmarks of acute inflammation and recently have been found to play a pivotal role in chronic inflammatory disease including cancer. Tumor-associated neutrophils are generally considered a pro-tumor factor in multiple types. Although neutrophils have been regarded as endpoint effector cells, increasing evidence has indicated properties of neutrophils for macrophage conversion. Neutrophils reprogrammed anti-parasitic macrophages toward M2 helminthcidal macrophages by their secretion of IL-13. Phagocytosis of apoptotic neutrophils inhibited inflammatory cytokine production in macrophages. We showed that myeloid CMTM4 KO M1 mice had different neutrophil infiltration in different types of cancer models. This finding leads to two important considerations; (1) Inflammation can be a key factor to decide neutrophil infiltration in the tumor microenvironment. (2) Infiltrated neutrophils can affect macrophage phenotypes in the inflammatory tumor microenvironment. We showed that the CMTM4 KO macrophages from inflammatory condition increased oxidative phosphorylation meaning M2-like functional phenotype compared to CMTM4 KO macrophages from transplanted tumors (FIG. 22e). It indicates that inflammation-mediated neutrophils may contribute to macrophage function. We further confirmed that depletion of neutrophils abolished the tumor-promoting function of CMTM4 KO macrophages in inflammation-mediated CRC development (FIGS. 22f and 22g) as well as recovered to M1-related gene expression (FIG. 23d-g) indicating that neutrophils can reprogram macrophages to tumor-promoting M1 macrophages having M2 functional phenotypes in an inflammatory environment. Thus, approaches to induce neutrophil apoptosis or migration may control the function of inflammatory macrophages.

In summary, we describe a critical role of CMTM4 in macrophage functions and a dual role of M1 macrophages in distinct types of tumorigenesis. Inflammation can affect neutrophils to reprogram macrophage function in the tumor microenvironment through metabolism control. Proper regulation of macrophages needs to be considered in distinct tumor types and conditions. Our results indicate that the modulation of CMTM4 may be a potential target for designing novel macrophage-mediated therapeutic strategies to improve cancer immune and metabolism therapy.

### c) Methods

### (1) Mice

C57BL/6 mice were purchased from Jackson Laboratories (Bar Harbor, ME). CMTM4^{tm1a(EUCOMM)Wtsi} mice were purchased from Wellcome Trust Sanger Institute (Hinxton Cambridge, UK). B6.129P2-Lyz2^{tm1(cre)lfo}/J, C57BL/6J-Apc^{Min}/J, and B6. FVB-Tg(MMTV-PyVT)634Mul/LellJ mice were obtained from Jackson Laboratories. Animal experiments were performed following the guidelines of the Houston Methodist Research Institute.

### (2) Induction of Colitis and Colorectal Cancer

To induce short-term colitis and inflammation studies, mice were given 2% DSS (molecular weight, 36-50 kDa; MP Biomedicals) for 5 days and sacrificed after treatment with regular drinking water for 5 days. For CAC models, mice were injected intraperitoneally with a single dose of AOM (10 mg/kg, Sigma-Aldrich). After 5 days, 2% DSS was given in the drinking water for 5 days, followed by 14 days of regular drinking water. The DSS treatment was repeated for two additional cycles, and mice were sacrificed 100 days after the first DSS treatment. To deplete neutrophils, 200 ug control IgG or a-Ly6G (1A8) were injected intraperitoneally every 3 days during DSS treatment. Body weights were recorded, and colon lengths and tumor sizes were measured in a blinded fashion. For AOM-induced CRC, AOM was injected intraperitoneally (10 mg/kg) once per week for 8 weeks. After 32 weeks, mice were assessed for the development of tumors.

### (3) Adoptive Transfer Experiments

5×10⁶ bone marrow cells from WT and CMTM4^{F/F}LysM^{cre} mice were inoculated into APC^{min/+} or MMTV-PvMT mice via tail vein injection after irradiation at 1300 rads. The APC^{min/+} mice were sacrificed 110-120 days after adoptive transfer and colons and small intestines were harvested. MMTV-PyMT mice were sacrificed 90-100 days after adoptive transfer and tumors were collected from whole bodies.

### (4) Determination of Clinical Colitis Scores

Weight loss, stool consistency, and any presence of occult or macroscopic blood were determined until mice were sacrificed. Stool consistency and rectal bleeding were analyzed; 0, normal stool; 1, soft but still formed stool; 2, loose stool; 3, mostly liquid stool; 4, diarrhea; 0, negative homoccult; 2, positive hemoccult; 4, blood traces visible in stool/rectal bleeding; and 0, >100% body weight; 1, 95-100% body weight; 2, 90-95% body weight; 3, 85-90% body weight; 4, <85% body weight.

### (5) Immunohistochemistry

Colons were removed from mice, flushed with cold PBS, opened longitudinally, fixed as “swiss roll” in 10% formalin solution at room temperature overnight, and embedded in paraffin. The paraffin-embedded colon sections were stained with hematoxylin and eosin (H&E) or Ki-67 and analyzed.

### (6) Antibodies and Flow Cytometry

Anti-Ly6C, anti-Ly6G, anti-CD11b, anti-CD45, anti-CD206, anti-iNOS, anti-CD3, anti-CD4, anti-CD8, anti-Foxp3, and isotype-matched mAbs were purchased from Affymetrix (San Diego, CA). Anti-Arginase was purchased from R&D Systems (Minneapolis, MN). Flow cytometric analyses were performed using FACSCanto II and FACSDiVa software (BD Biosciences).

### (7) ELISA

Cytokine concentrations in culture supernatants and mouse serum were measured with mouse IL-6, IL-12p40, and TNF-α ELISA kits (Affymetrix) as per the manufacturer's instructions.

### (8) Western Blot Analysis

Protein samples were separated on sodium dodecyl sulfate (SDS)-polyacrylamide gels and transferred to PVDF membranes. The membranes were blocked in 4% skim milk solution, incubated with an appropriate antibody, and subsequently incubated with a secondary antibody conjugated to horseradish peroxidase. The antibodies for p-STAT1, p-ERK1/2, p-p38, p-SAPK/JNK, p-STAT5, p-STAT6, p-Akt, and p-STAT3 were purchased from cell signaling technology, Inc (Beverly, MA) and the antibodies for CMTM4 and actin were purchased from Santa Cruz Biotechnology. The immunoreactive bands were visualized with the ECL system (Thermo Scientific).

### (9) Real-Time PCR

RNA was isolated from organs using Trizol (Invitrogen; Carlsbad, CA) per the manufacturer's specifications. The cDNA was synthesized from 1 μg of total RNA using M-MLV reverse transcriptase (Promega, Madison, WI) and qPCR was performed in 384-well plates using FastStart SYBR Green Master Mix (Roche, Mannheim, Germany) on a ViiA™ 7 real-time PCR system (Applied Biosystems, Foster City, CA). After cDNA amplification, samples were normalized to GAPDH, and data were expressed as mean±SD of triplicates.

### (10) Transcriptome Analysis

Macrophages from three different CMTM4^{F/F} and CMTM4^{F/F}LysM^{cre} mice were treated with 10 ng/ml IFNγ and 10 ng/ml LPS or 20 ng/ml IL-4 for 1 hour and were subjected for RNAseq analysis. RNA was hybridized to mouse Illumina Platform PE150. Probes with no expression were removed. Differential gene expression analysis was performed using the limma R package and significantly differentially expressed genes were identified on p-value <0.05 with Benjamini & Hochberg false-discovery rate correction. Heatmaps of the top 100 differentially expressed genes were created using the gplots R package and unsupervised hierarchical clustering was done based on Euclidean distance. Gene ontology enrichment analysis and visualization were performed.

Single-cell suspensions from TIL and lamina propria (LP) leukocytes were loaded onto a Chromium Single Cell Chip (10× Genomics) according to the manufacturer's instructions for co-encapsulation with barcoded Gel Beads at a target capture rate of ˜5000 individual cells per samples. We captured mRNA was barcoded during cDNA synthesis and converted the barcoded cDNA into pooled single-cell RNA-seq libraries for Illumina sequencing using the Chromium Single Cell 3′ Solution (10× Genomics) according to the manufacturer's instructions. All samples were processed simultaneously with the Chromium Controller (10× Genomics) and the resulting libraries were prepared in parallel in a single batch. We pooled all libraries for samples, each of which was barcoded with a unique Illumina sample index, for sequencing in a single Illumina flow cell. All libraries were sequenced, barcoded with an 8-base index read, a 26 base read 1 containing cell identifying barcodes and unique molecular identifiers, and a 98 base read 2 containing transcript sequences on an Illumina HiSeq 4000. The online data analysis tool Ingenuity pathway analysis (IPA) was used using the genes that had a p-value <0.05 and a >2-fold change (FC) difference between WT and CMTM4 KO macrophages or LP and TIL. Core analysis was run on these data set to determine the pathways most affected by the loss of CMTM4 or by inflammation.

### (11) Statistical Analysis

Statistical analyses were performed using Student's t-test or one-way ANOVA in GraphPad Prism 9. It was represented as mean z SD. P<0.05 was considered statistically significant.

### 3. Example 3

### a) Results

### (1) Identification of the CMTM4 Functional Domains.

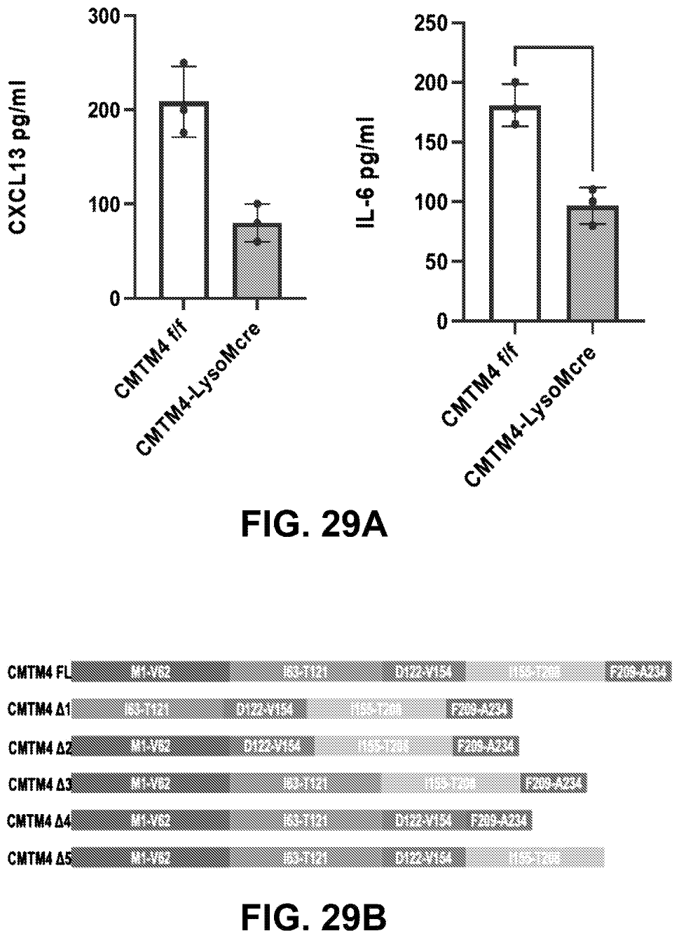

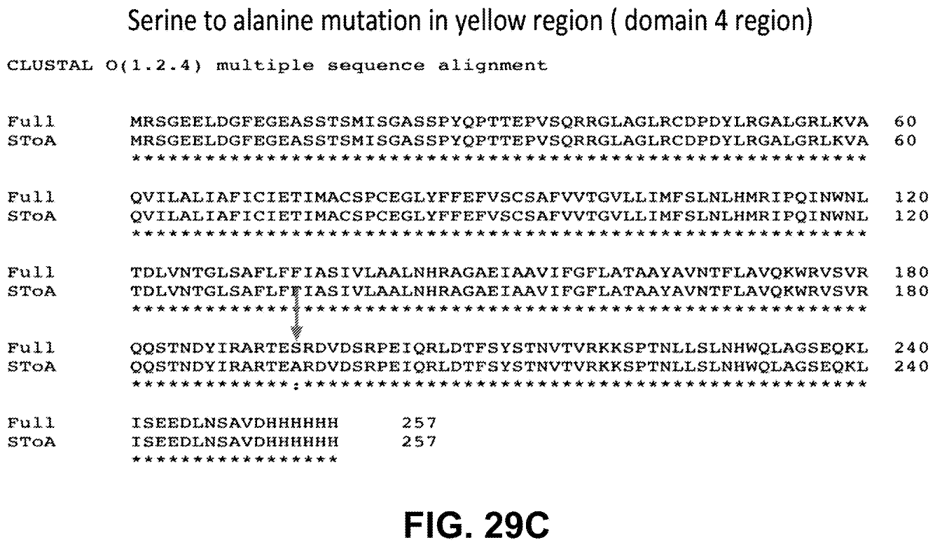

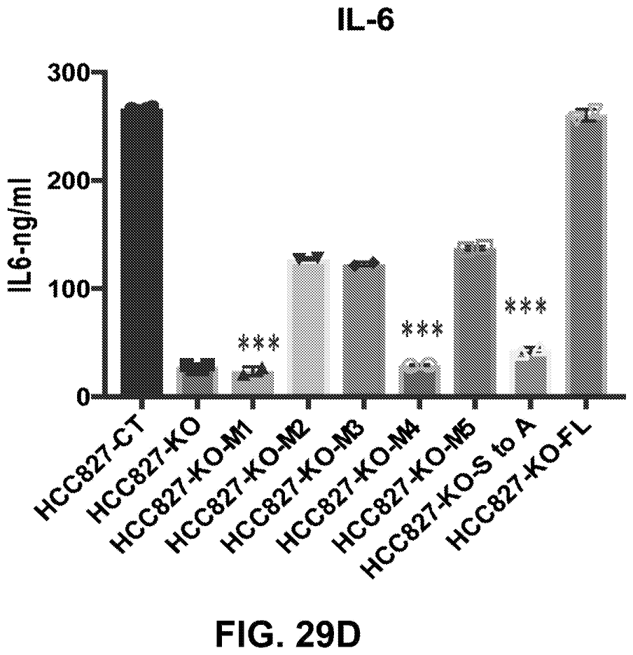

We aimed to determine which domain of CMTM4 mediates its function. In our in vivo experiments, we observed that CMTM4 f/f (control wildtype) mice had higher levels of interleukin-6 (IL-6) and CXCL13 in the blood when compared to CMTM4 f/fxLysMcre mice (FIG. 29A). We also measured IL-6 levels in cell supernatant collected from HCC827-CMTM4-CT and HCC827-CMTM4-KO cells in vitro. We found that IL-6 secretion was lower in the supernatant collected from HCC827-CMTM4-KO cells compared to HCC827-CMTM4-CT cells (FIG. 29A). IL-6 belongs to a broad class of cytokines that is involved in the regulation of various homeostatic and pathological processes, ranging from regulating embryonic development, wound healing and ageing, inflammation, and immunity. IL-6 signaling pathway also plays a role in cancer biology, e.g., cancer cell invasiveness and metastasis formation. Based on these results, we further conducted experiments to identify the specific CMTM4 domain that is essential for IL-6 secretion. These investigations contribute to a better understanding of CMTM4's role in cancer progression and provide insights into its use as a therapeutic target, particularly in combination with other treatments.

### (2) CMTM4 Domain M1 (Red 1-62) and M4 (Yellow 155-208) Regulates IL-6 Secretion

The human CMTM4 coding sequences were synthesized using the GeneArt Gene Synthesis service provided by Thermo Fisher Scientific. The transmembrane domains of CMTM4 (CMTM4 Full length, CMTM4 AM1, CMTM4 AM2, CMTM4 AM3, CMTM4 AM4, CMTM4 AM5 and CMTM4 with point mutation from 194 Ser to Ala in domain 4 (M4 region)) (FIGS. 29B and 29C) were cloned into the pMSCV-puro expression vector. These constructs were inserted into a retroviral vector that expresses the puromycin-selection marker. To ensure the accuracy of the constructs, all sequences were verified by sequencing.

To produce viruses for transduction of human cells, Phoenix-Ampho cells were employed. The cells were transfected with the CMTM4-His Tag-pMSCV-puro vector containing the coding sequences of the respective proteins or an empty vector (EV). Transfection was carried out using Lipofectamine 2000 (Invitrogen). After 24 hours of transfection, fresh media was added, and the transfected cells were grown for an additional 48 hours.

Virus-containing supernatants were collected after 48 hours, filtered through a 0.2 μm filter, and then added to the HCC827-CMTM4-KO cells in the presence of 10 μg/ml polybrene. The cells were then spun at 2,500 rpm for 45 minutes to enhance viral transduction. The transduced cells were cultivated for 14 days in the presence of 12 μg/ml puromycin to select for stably transfected cells. The expression of the transfected genes was verified by IP. We lysed the cells and pulldown with anti His Ab and prob with anti-CMTM4 Ab, confirming the successful introduction and expression of the CMTM4 in the HCC827-CMTM4 KO cells.

Cell culture supernatant was collected from HCC827-CT, HCC827-CMTM4-KO transduced cells to assess IL-6 level. We found that HCC827-CMTM4-KO cells transuded with M2, M3 or M5 domain deleted CMTM4 partially restored IL-6 secretion; however CMTM4 KO cells transduced with domain M1 or M4 domain failed to restore IL-6 secretion. The results indicate that domain M1 (red region) and M4 (yellow region) are required for IL-6 secretion regulated by CMTM4. Moreover, we performed a single amino acid mutation (from a.a.194 serine to alanine) in CMTM4 domain M4. We found that CMTM4 with a serine to alanine mutation at a.a. 194 in domain M4 also failed to restore IL-6 secretion in CMTM4 KO cells (FIGS. 29C and 29D). We conclude that Ser 194 in domain M4 of CMTM4 is essential for the regulation of IL-6 secretion by CMTM4.

(3) CMTM4-KO Inhibits ER Stress Gene Expression in M1 and M2 Macrophage and CMTM4 Associates with ER Stress Downstream Activation Proteins TRAF2 and TRAF6

Cancer cells exhibit a high growth rate, leading to a sustained and increased demand for de novo protein synthesis, folding, and maturation. Hostile environmental conditions, such as hypoxia, oxidative stress, and chemotherapy, pose a threat to proper protein folding in the endoplasmic reticulum (ER), resulting in ER stress (ERS). In mammalian cells, the unfolded protein response (UPR) is triggered by three ER transmembrane proteins that act as ER stress sensors: inositol-requiring enzyme 1α (IRE1α), activating transcription factor 6 (ATF6), and protein kinase RNA-like ER kinase (PERK). However, persistent ER stress is associated with the adaptation of malignant cells in the tumor microenvironment (TME) and can disrupt ER homeostasis in cancer and immune cells. Macrophages, as a key component of the innate immune system, play a crucial role in maintaining tissue homeostasis and immunity. Macrophages can be further categorized into M0 (naive), M1 macrophages (proinflammatory), and M2 macrophages (immunosuppressive). A recent study published in Nature Immunology discovered that the PERK arm of UPR signaling enhances the metabolic functions of macrophages and promotes an immunosuppressive M2 macrophage phenotype.

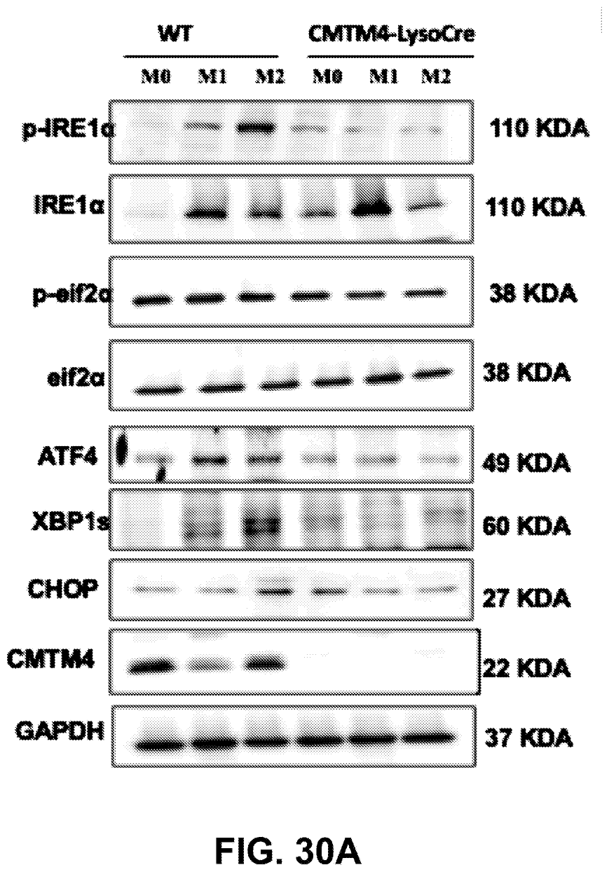

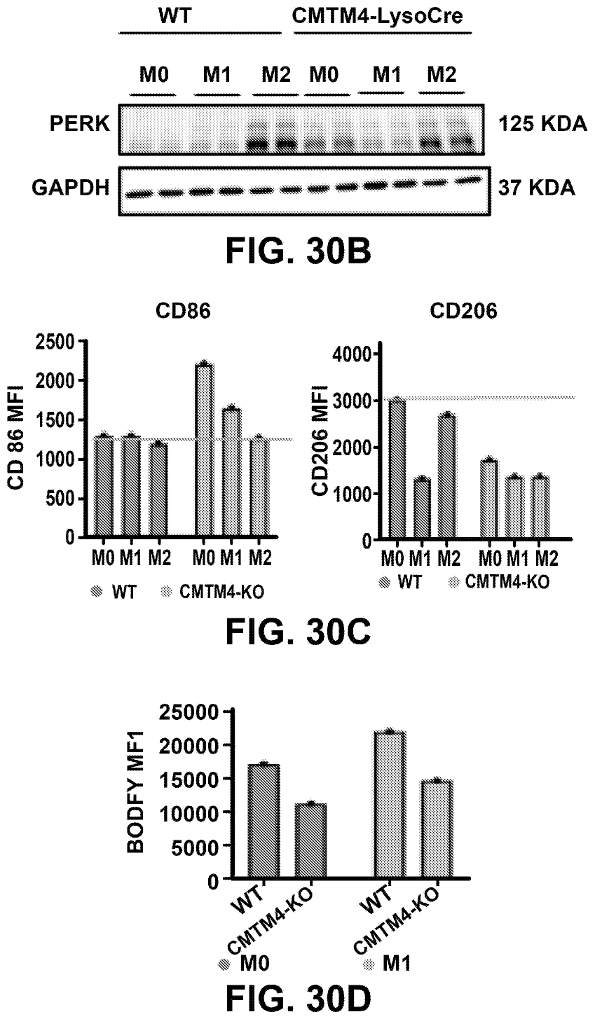

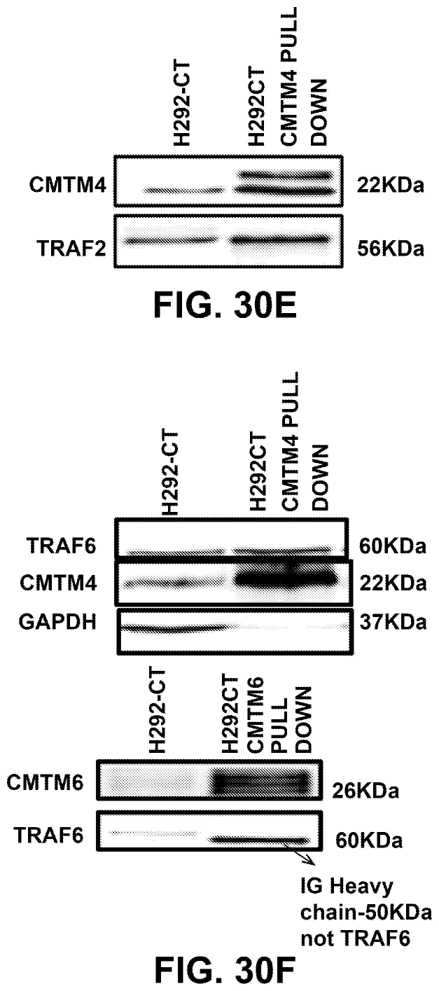

To gain mechanistic insights, we stimulated bone marrow-derived macrophages (BMDMs) isolated from WT and CMTM4f/f-LysMcre mice with lipopolysaccharide (LPS)+ interferon-gamma (IFN-γ, M1 macrophage) and IL-4 (M2 macrophage) cytokines and performed western blot analysis. The results showed that macrophages isolated from WT mice, stimulated with IL-4 (M2) exhibited high levels of PERK compared to macrophages activated with LPS+IFN-γ (M1), which showed low levels of PERK activation. However, BMDMs isolated from CMTM4f/f-LysMcre mice showed a lower levl of PERK/eIF2α/ATF4 activation in both M1 and M2 macrophages. Activation of PERK signaling promotes the development of immunosuppressive M2 macrophages; however, deletion of CMTM4 attenuated this effect (FIGS. 30A and 30B). We also observed downregulation of the ER stress pathway, specifically IRE1 and its downstream target XBP1, in CMTM4f/f-LysMcre BMDMs in both M1 and M2 macrophages as compared with WT mice (FIG. 30A). Moreover, the ER stress marker CHOP was also downregulated in CMTM4f/f-LysMcre BMDMs compared to WT cells (FIG. 30A). We further confirmed that in M1 macrophages culture condition, CMTM4f/f-LysMcre BMDMs expressed a higher level of M1 gene signature, e.g., CD86 and lower M2 genes e.g., CD206 expression compared to WT control, indicating an M1-like phenotype (FIG. 30C). This supports our finding that CMTM4 deletion promoted macrophage polarization toward a proinflammatory M1-like phenotype and prevent the M2 macrophage differentiation. Overall, these results indicate that CMTM4 can regulate the ER stress activation. The CMTM4 KO or blockade can reduce the ER Stress activation.

TRAF2 is downstream of ER Stress activation signaling and involved in various cancer-relevant cellular processes, such as the activation of transcription factors of the NFκB family, stimulation of mitogen-activated protein (MAP) kinase cascades, endoplasmic reticulum (ER) stress signaling, autophagy, and the control of cell death programs. Tumor necrosis factor receptor-associated factor-6 (TRAF6) is a ubiquitin E3 ligase and plays an important role in tumor invasion and metastasis. To determine whether CMTM4 interact with TRAR2 and TRAF6, we performed a Co-IP experiment and found strong interaction between CMTM4 and TRAF2 and TRAF6 In contrast, CMTM6 does not have any interaction with TRAF6 (FIGS. 30E and 30F).

In recent years, increasing evidence indicates that ER stress and UPR activation can regulate cellular processes beyond ER protein folding and play crucial roles in lipid metabolism. ER stress, caused by disruption in ER protein-folding capacity, triggers the activation of an evolutionarily conserved UPR signaling system to restore ER homeostasis. Accumulating evidence indicates that UPR pathway activation can modulate lipid metabolism by controlling the transcriptional regulation of lipogenesis. Next, we investigated whether CMTM4 deletion also inhibits lipid biogenesis by suppressing ER stress. We cultured WT and CMTM4f/f-LysMcre BMDMs and stained them with BODIPY followed by flow cytometric analysis and found that CMTM4f/f-LysMcre macrophages exhibited lower lipid content compared to WT control (FIG. 30D). Overall, these results indicate that CMTM4 plays a role in the regulation of ER stress and ER stress-mediated regulation of lipogenesis.

(4) CMTM4 Interacts with TRAF2 and RAB35/21 Proteins.

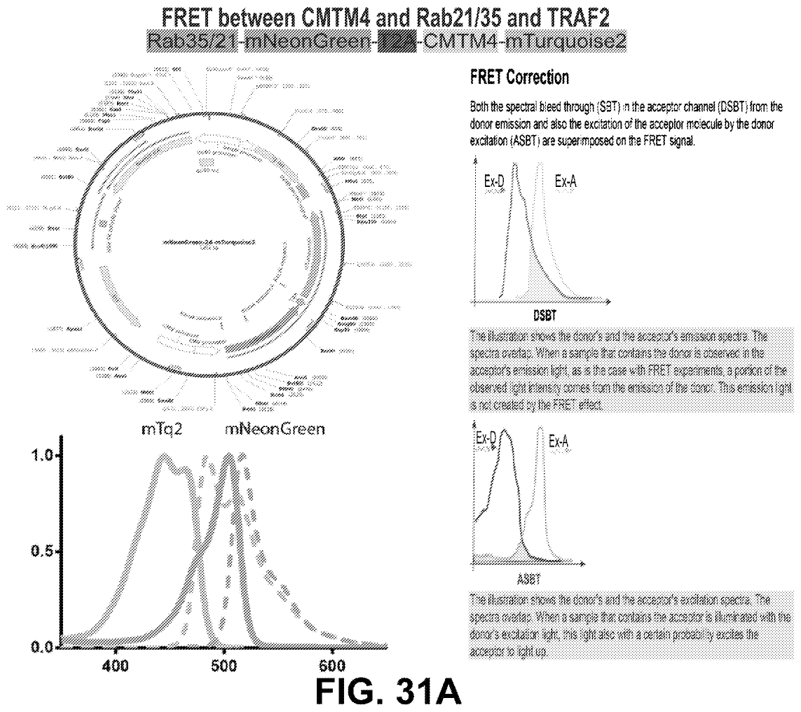

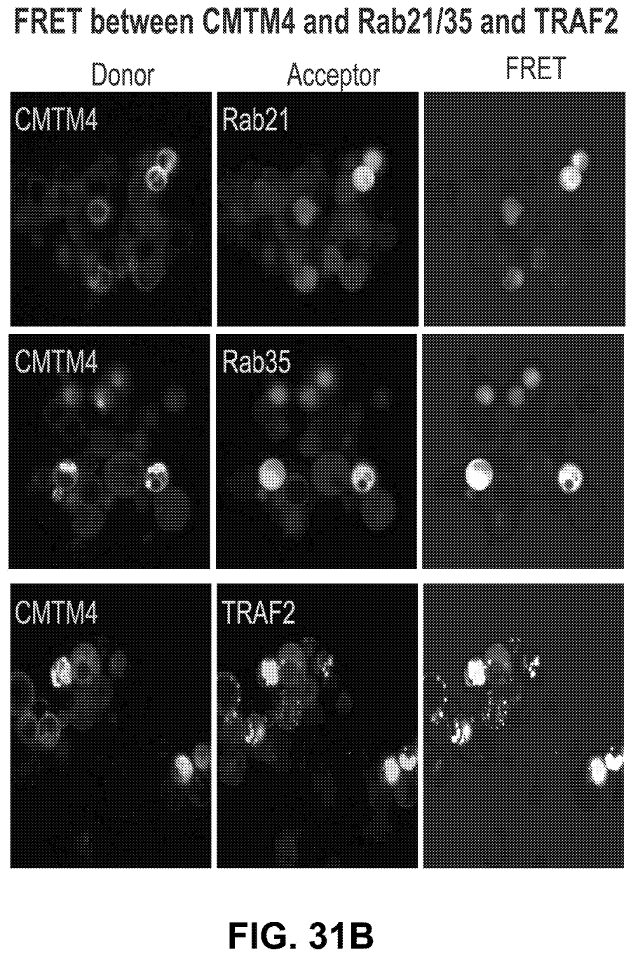

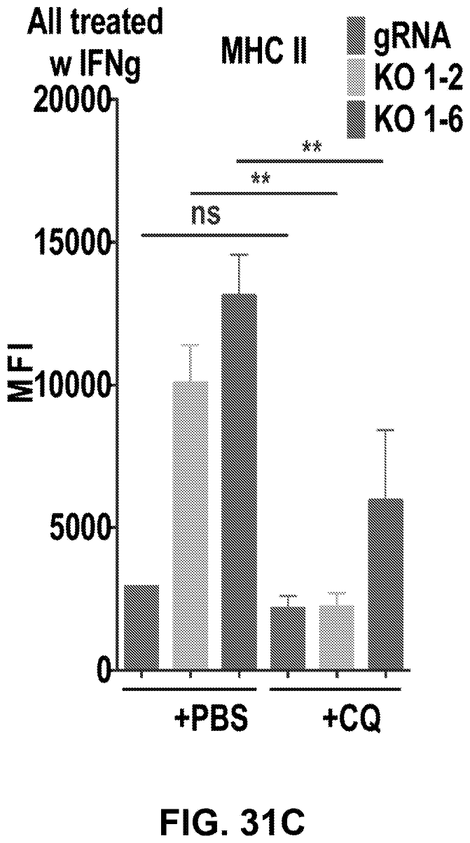

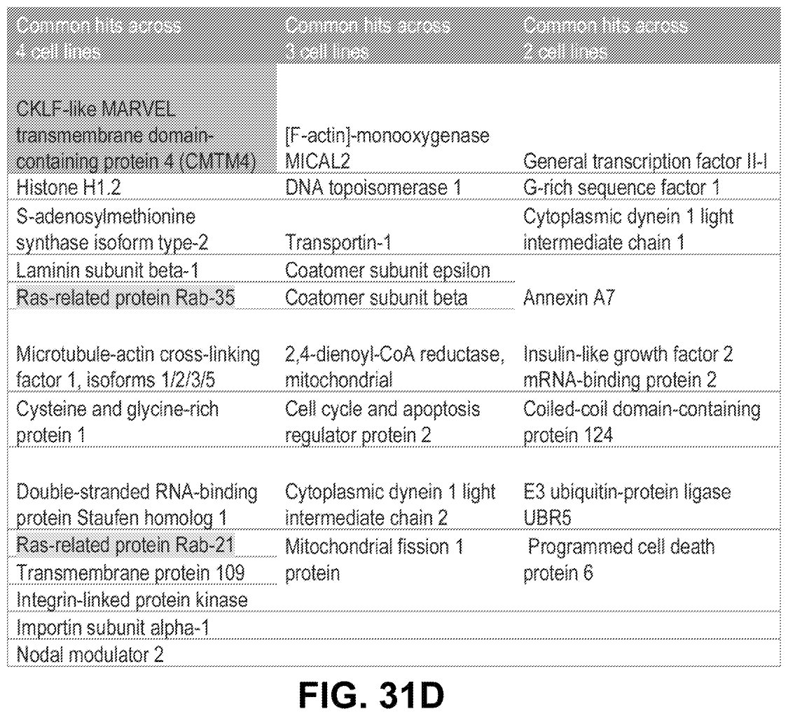

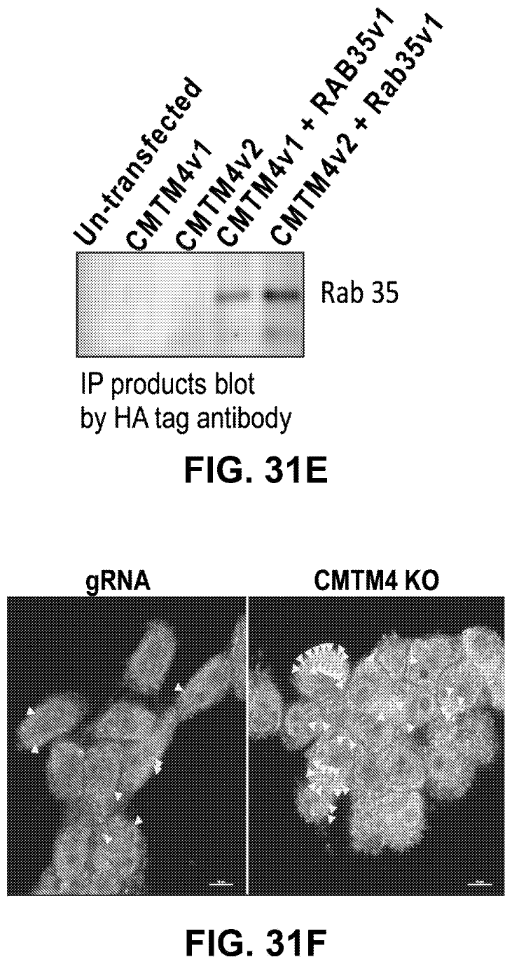

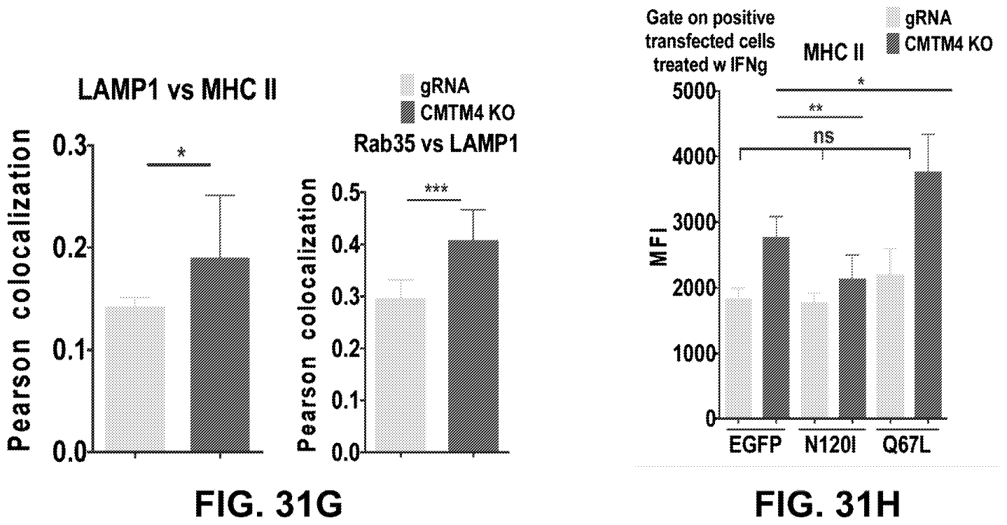

We further performed a mass spec analysis of CMTM4 associated protein by pull-down and identified candidate proteins that associate with CMTM4. Rab3 and Rab, 21 has repeated shown up in multiple tumor lines (FIG. 31D). We further evaluated the association of CMTM4 with these proteins, e.g., Rab35, Rab21 and TRAF2, by the FRET (Fluorescence Resonance Energy Transfer) assay (FIG. 31A). As shown in FIG. 31B that CMTM4 was closely associate with Rab21, TRAF2 and Rab35, which have changed the blue (donor) and green (acceptor protein) to yellow color due to the proximity of two proteins. We further confirmed the interaction of CMTM4 with Rab35 by the Co-IP experiment (FIG. 31E). The Rab35 is well known to control the protein recycling. Tumor antigen and MHC Class 11 are assembled in the lysosome followed by presentation of the antigen to the cell surface. The CMTM4 deletion results in reduced association of RAB 35 with CMTM4, thereby reducing protein recycle and favoring the lysosome pathway, thus higher MHC Class II assembling and surface expression upon IFNγ stimulation. This effect (i.e., higher levels of surface MHC class 11 in CMTM4 KO cells) was abolished by chloroquine (lysosome breaker) as shown in FIG. 31C. This effect was further confirmed by confocal microscopy. We found that CMTM4 KO tumor cells had higher MHC class II or Rab35 protein co-localization wit lysosomal protein LAMP1 r (FIGS. 31F and 31G). The upregulation of MHC Class II in CMTM4 KO cells was abolished by overexpression of dominant negative RAB35 mutant (N1201) in CMTM4 wildtype tumor cells, but was further enhanced by overexpression of constitutive active form of RAB35 (Q67L).

(5) CMTM4 KO Enhances the MHC Class IT Expression, Promotes the T Cells Proliferation/Activation, Reduces the Tumor Size and Prolongs the Survival of Tumor Bearing Mice Treated with TCR Transgenic T Cell Therapy.

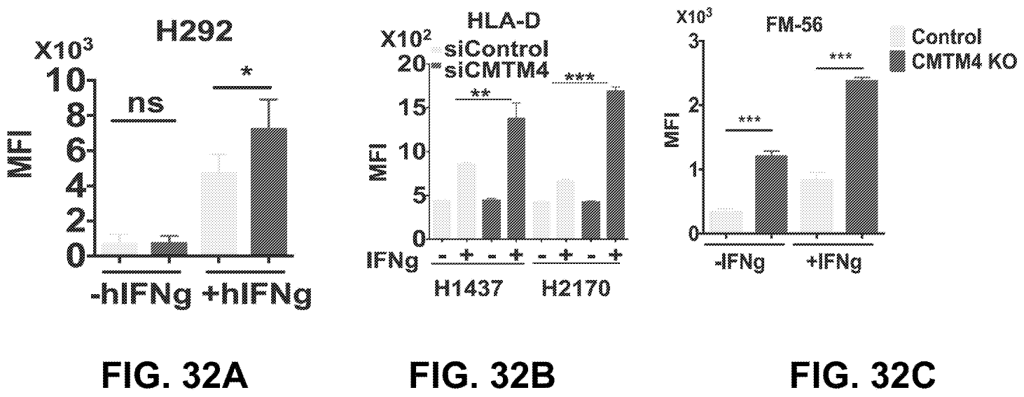

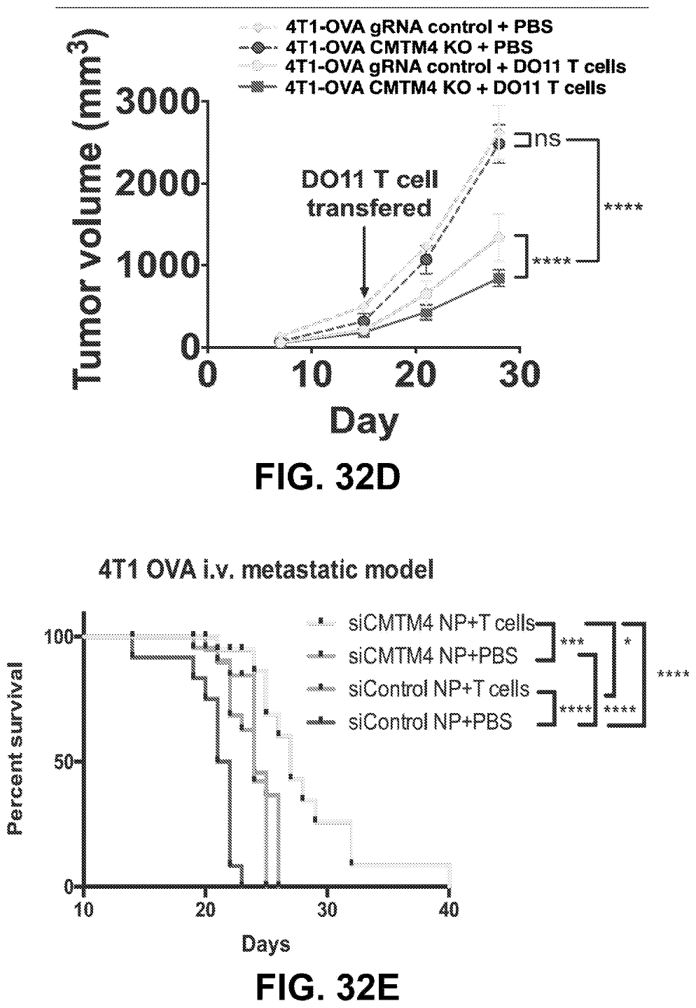

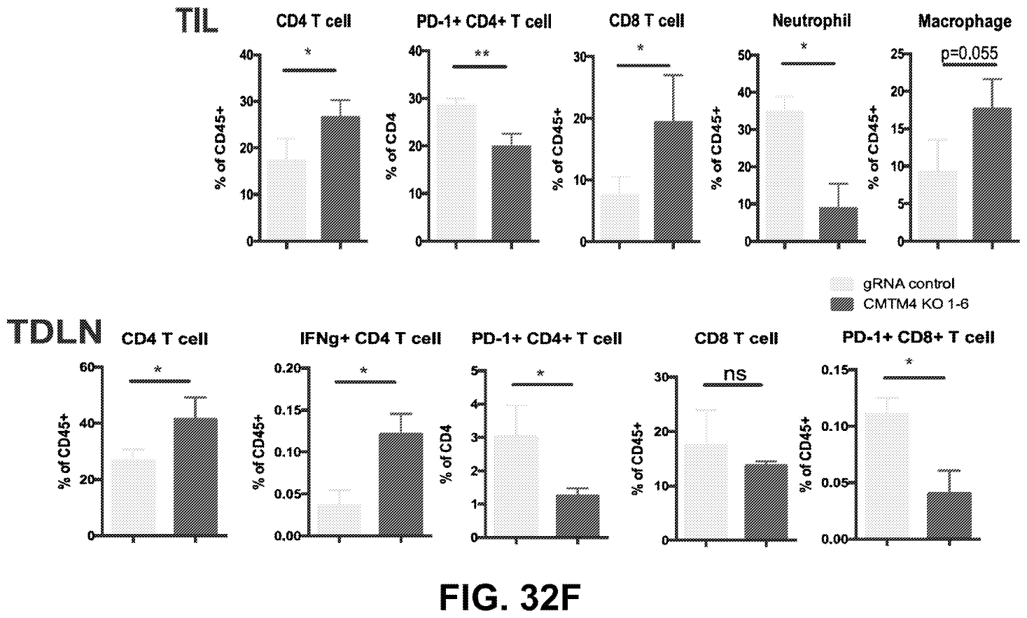

We confirmed the upregulation of MHC Class II in multiple CMTM4 KO human cancer cell lines, e.g., lung cancer cell, H292, H1437 and H2170 and melanoma cells line FM-56 (FIG. 32A-32C). We further tested the immunological consequence of upregulation of MHC 11 by CMTM4 KO with OVA CD4 TCR T cell therapy (DO11T) in combination with liposome CMTM4 siRNA delivery in mammary fat subcutaneous and metastatic iv infusion of OVA-4T1 tumor models. The results indicate the TCR T cell in conjunction with liposome mediated siRNA KO CMTM4 can significantly reduce the tumor size and prolong the survival than TCR T cell alone or siRNA CMTM4 KD alone (FIG. 32D, E). We also observed a higher percentage of activated CD8 and CD4 infiltrating (IFNγ positive cells) in the tumor site and draining lympho-nodes and a lower percentage of exhausted T cells (PD-1 positive T cells) and reduced neutrophil infiltration in the tumor (FIG. 32F). This supports that downregulation CMTM4 expression in the tumor can facilitate T cell therapy, cancer vaccine, and immune checkpoint therapy.

### (6) Compound Screening for IL-6 and ER Stress and CMTM4 Inhibition.

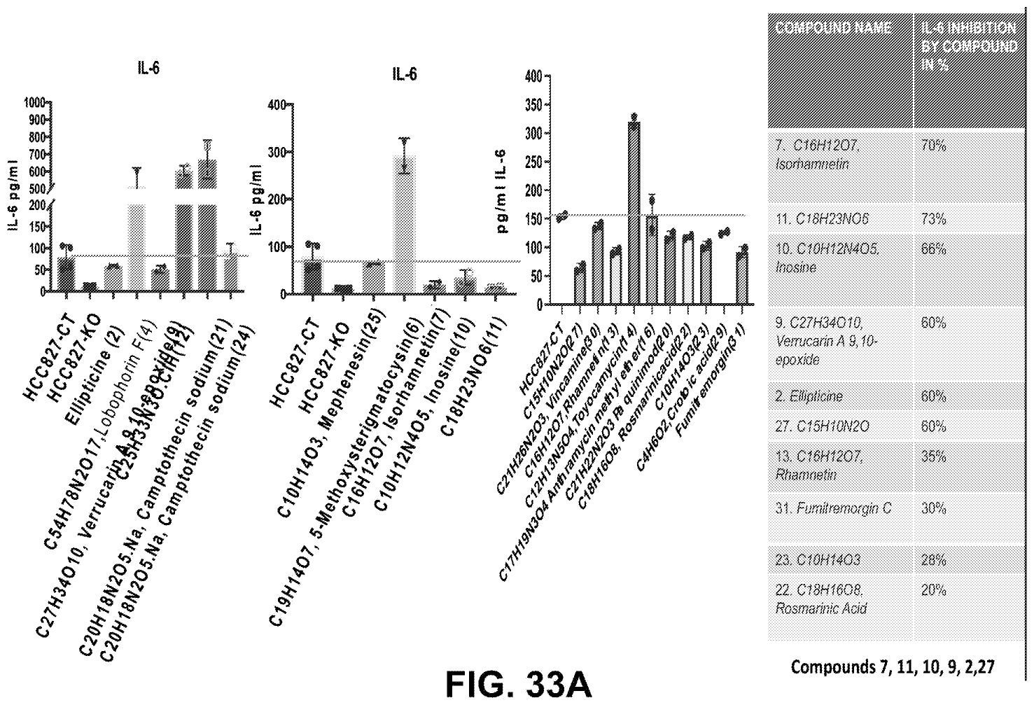

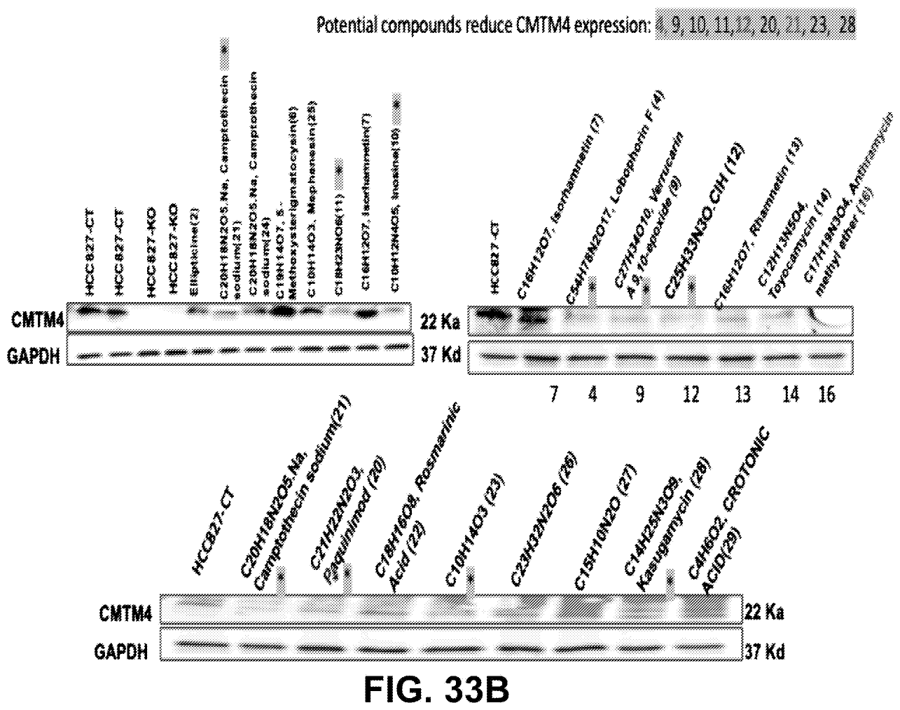

To develop therapeutics that can target CMTM4 directly or the CMTM4-regulated pathways, we have screened small compounds that can inhibit ER-stress, IL-6 and CMTM4 expression. We screened 21 compounds by evaluating their effect on IL-6 secretion. We treated the HCC827-CT cells at 1 μm concentration of small compounds for 24 hrs. followed by collecting cell supernatants for IL-6 measurement by ELISA. Ten compounds inhibited IL-6 secretion (FIG. 33A). We then performed western blot to determine whether these compounds also inhibited CMTM4 expression. The result showed that 5 compounds (Ellipticine, C10H12N4O5, Inosine, C18H23NO6, C20H18N2O5·Na, Camptothecin sodium, C9H16N2O6, Tetrahydro uridine) decreased CMTM4 protein expression (FIG. 33B).

### (7) CMTM4 Regulates Akt/mTOR Signaling in Human Cancer and Deletion of CMTM4 Sensitizes Human Cancer Cells for EGFR Inhibition.

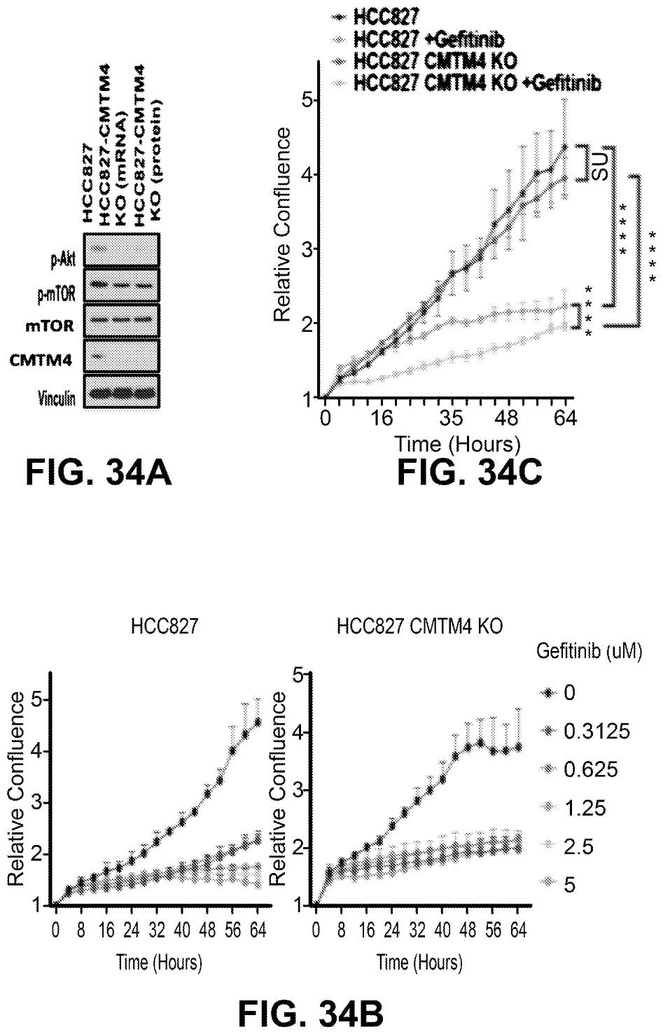

To confirm our findings in the human system, we used the human lung cancer cell line HCC827 with exon 19 deletion in EGFR, which is an activating mutation that results in excess EGFR expression. We confirmed CMTM4 expression in this cell and generated CRISPR knockout lines using Cas9 mRNA or protein (FIG. 34). Loss of CMTM4 significantly reduced pAKT/mTOR signaling (FIG. 34A), which is consistent with our findings in multiple tumor cell lines. To explore the therapeutic potential of CMTM4, we evaluated the effect of the EGFR tyrosine kinase inhibitor, gefitinib, on CMTM4 KO cells. HCC827 cells with CMTM4 KO grew at a rate similar to its parental control and both were sensitive to gefitinib treatment at higher drug concentrations (FIGS. 34B and 34C). However, CMTM4 KO cells show elevated sensitivity to gefitinib treatment (FIGS. 34B and 34C), indicating CMTM4 plays a role in tumor intrinsic resistance to EGFR tyrosine kinase inhibitors. Our data suggests CMTM4 is a novel regulator of the EGFR/AKT/mTOR pathway in human tumor and affects sensitivity to EGFR tyrosine kinase inhibition.

### b) Material and Method

### (1) DNA Cloning and Viral Transduction

The human CMTM4 coding sequences were synthesized using the GeneArt Gene Synthesis service provided by Thermo Fisher Scientific. The transmembrane domains of CMTM4 (CMTM4 Full, CMTM4 M1, CMTM4 M2, CMTM4 M3, CMTM4 M4, CMTM4 M5 and CMTM4 Ser to Ala) were cloned into the pMSCV-puro expression vector. These constructs were inserted into a retroviral vector that expresses the puromycin-selection marker. To ensure the accuracy of the constructs, all sequences were verified by sequencing.

To produce viruses for transduction of human cells, Phoenix-Ampho cells were employed. The cells were transfected with the CMTM4-His Tag-pMSCV-puro-vector containing the coding sequences of the respective proteins or an empty vector (EV). Transfection was carried out using Lipofectamine 2000 (Invitrogen). After 24 hours of transfection, fresh media was added, and the transfected cells were grown for an additional 48 hours.

Virus-containing supernatants were collected after 48 hours, filtered through a 0.2 μm filter, and then added to the target cells in the presence of 10 μg/ml polybrene. The cells were then spun at 2,500 rpm for 45 minutes to enhance viral transduction. The transduced cells were cultivated for 14 days in the presence of 12 μg/ml puromycin to select for stably transfected cells. The expression of the transfected genes was verified by immunoblotting, confirming the successful introduction and expression of the desired genes in the cells.

### (2) Immunoprecipitation and Wester Blotting

HCC827-CT cells were cultured in a 100-mm dish. The cells were washed with serum-free DMEM (Dulbecco's Modified Eagle Medium) and then solubilized in REPA lysis buffer. The cell lysates were incubated on ice for 30 minutes and subsequently cleared by centrifugation at 21,130 g for 30 minutes at 2° C. A portion of the lysates was mixed with reducing SDS sample buffer containing 50 mM DTT (Dithiothreitol). This portion can be used for direct analysis by immunoblotting. The remaining portion of the sample was subjected to immunoprecipitation. For this, 10 μl of anti-His antibody and Protein A/G magnetic beads (Sigma) were added to the lysates and incubated overnight. The antibody and beads selectively bind to the protein of interest, in this case, the His-tagged protein. After incubation, the magnetic beads were washed three times with 0.1% PBST (Phosphate-Buffered Saline with Tween 20) containing lysis buffer to remove any non-specifically bound proteins. Finally, the bound proteins were eluted from the magnetic beads by heating the samples to 94° C. for 5 minutes in SDS sample buffer with 50 mM DTT. The samples obtained from both direct analysis and immunoprecipitation were then analyzed by immunoblotting, a technique used to detect and visualize specific proteins of interest.

### E. REFERENCES

Ahmad, R., Sylvester, J., Ahmad, M. & Zafarullah, M. Adaptor proteins and Ras synergistically regulate IL-1-induced ADAMTS-4 expression in human chondrocytes. J Immunol 182, 5081-5087 (2009).
Akhurst, R. J. & Derynck, R. TGF-beta signaling in cancer—a double-edged sword. Trends Cell Biol 11, S44-51 (2001).
Almand B, Clark J I, Nikitina E, van Beynen J, English N R, Knight S C, et al. Increased production of immature myeloid cells in cancer patients: a mechanism of immunosuppression in cancer. J Immunol 2001; 166:678-89
Arango Duque, G. & Descoteaux, A. Macrophage cytokines: involvement in immunity and infectious diseases. Front Immunol 5, 491 (2014).
Atreya, R. & Neurath, M. F. Signaling molecules: the pathogenic role of the IL-6/STAT-3 trans signaling pathway in intestinal inflammation and in colonic cancer. Curr Drug Targets 9, 369-374 (2008).
Barbieri M A, Fernandez-Pol S, Hunker C, Horazdovsky B H, Stahl P D. Role of rab5 in EGF receptor-mediated signal transduction. Eur J Cell Biol 2004; 83:305-14
Beatty, G. L. et al. CD40 agonists alter tumor stroma and show efficacy against pancreatic carcinoma in mice and humans. Science 331, 1612-1616 (2011).
Benoit, M., Desnues, B. & Mege, J. L. Macrophage polarization in bacterial infections. J Immunol 181, 3733-3739 (2008).
Bierie, B. & Moses, H. L. Tumour microenvironment: TGFbeta: the molecular Jekyll and Hyde of cancer. Nat Rev Cancer 6, 506-520 (2006).
Biswas, S. K. & Mantovani, A. Macrophage plasticity and interaction with lymphocyte subsets: cancer as a paradigm. Nat Immunol 11, 889-896 (2010).
Bobrovnikova-Marjon E, Hatzivassiliou G, Grigoriadou C, et al. PERK-dependent regulation of lipogenesis during mouse mammary gland development and adipocyte differentiation. Proceedings of the National Academy of Sciences of the United States of America. 2008; 105(42):16314-16319
Buck M D, Sowell R T, Kaech S M & Pearce E L Metabolic Instruction of Immunity. Cell 169, 570-586 (2017).
Burnett S H, Kershen E J, Zhang J, Zeng L, Straley S C, Kaplan A M, et al. Conditional macrophage ablation in transgenic mice expressing a Fas-based suicide gene. J Leukoc Biol 2004; 75:612-23
Casaletto J B, McClatchey A I. Spatial regulation of receptor tyrosine kinases in development and cancer. Nat Rev Cancer 2012; 12:387-400
Ceresa B P. Regulation of EGFR endocytic trafficking by rab proteins. Histol Histopathol 2006; 21:987-93
Chang, H. R. et al. Role of LpL (Lipoprotein Lipase) in Macrophage Polarization In Vitro and In Vivo. Arterioscler Thromb Vasc Biol 39, 1967-1985 (2019).
Chen, F. et al. Neutrophils prime a long-lived effector macrophage phenotype that mediates accelerated helminth expulsion. Nat Immunol 15, 938-946 (2014).
Chen, H. M. et al. Blocking immunoinhibitory receptor LILRB2 reprograms tumor-associated myeloid cells and promotes antitumor immunity. J Clin Invest 128, 5647-5662 (2018).
Coffelt, S. B., Wellenstein, M. D. & de Visser, K. E. Neutrophils in cancer: neutral no more. Nat Rev Cancer 16, 431-446 (2016).
Colonna, M. TREMs in the immune system and beyond. Nat Rev Immunol 3, 445-453 (2003).
Coussens L M, Zitvogel L, Palucka A K. Neutralizing tumor-promoting chronic inflammation: a magic bullet? Science 2013; 339:286-91
Crusz S M, Balkwill F R. Inflammation and cancer: advances and new agents. Nat Rev Clin Oncol 2015; 12:584-96
Cullis D N, Philip B, Baleja J D, Feig L A. Rab 11-FIP2, an adaptor protein connecting cellular components involved in internalization and recycling of epidermal growth factor receptors. J Biol Chem 2002; 277:49158-66
Davidov, V., Jensen, G., Mai, S., Chen, S. H. & Pan, P. Y. Analyzing One Cell at a TIME: Analysis of Myeloid Cell Contributions in the Tumor Immune Microenvironment. Front Immunol 11, 1842 (2020).
Di Caro, G. et al. Dual prognostic significance of tumour-associated macrophages in human pancreatic adenocarcinoma treated or untreated with chemotherapy. Gut 65, 1710-1720 (2016).
Dong, H. et al. Lactoferrin-containing immunocomplex mediates antitumor effects by resetting tumor-associated macrophages to M1 phenotype. J Immunother Cancer 8 (2020).
Etchegaray J-P & Mostoslavsky R Interplay between Metabolism and Epigenetics: A Nuclear Adaptation to Environmental Changes. Molecular Cell 62, 695-711 (2016).
Eyles J L, Roberts A W, Metcalf D, Wicks I P. Granulocyte colony-stimulating factor and neutrophils—forgotten mediators of inflammatory disease. Nat Clin Pract Rheumatol 2006; 2:500-10
Fadok, V. A. et al. Macrophages that have ingested apoptotic cells in vitro inhibit proinflammatory cytokine production through autocrine/paracrine mechanisms involving TGF-beta, PGE2, and PAF. J Clin Invest 101, 890-898 (1998).
Fridman, W. H., Pages, F., Sautes-Fridman, C. & Galon, J. The immune contexture in human tumours: impact on clinical outcome. Nat Rev Cancer 12, 298-306 (2012).
Gao, C. H., Dong, H. L., Tai, L. & Gao, X. M. Lactoferrin-Containing Immunocomplexes Drive the Conversion of Human Macrophages from M2- into M1-like Phenotype. Front Immunol 9, 37 (2018).
Greten F R, Grivennikov S I. Inflammation and Cancer: Triggers, Mechanisms, and Consequences. Immunity 2019; 51:27-41
Grivennikov, S. I. & Karin, M. Dangerous liaisons: STAT3 and NF-kappaB collaboration and crosstalk in cancer. Cytokine Growth Factor Rev 21, 11-19 (2010).
Grivennikov, S. I., Greten, F. R. & Karin, M. Immunity, inflammation, and cancer. Cell 140, 883-899 (2010).
Gupta, J. et al. Dual function of p38alpha MAPK in colon cancer: suppression of colitis-associated tumor initiation but requirement for cancer cell survival. Cancer Cell 25, 484-500 (2014).
Hagerling, C., Casbon, A. J. & Werb, Z. Balancing the innate immune system in tumor development. Trends Cell Biol 25, 214-220 (2015).
Huang B, Pan P Y, Li Q, Sato A I, Levy D E, Bromberg J, et al. Gr-1+CD115+ immature myeloid suppressor cells mediate the development of tumor-induced T regulatory cells and T-cell anergy in tumor-bearing host. Cancer Res 2006; 66:1123-31
Kim, S. J., Im, Y. H., Markowitz, S. D. & Bang, Y. J. Molecular mechanisms of inactivation of TGF-beta receptors during carcinogenesis. Cytokine Growth Factor Rev 11, 159-168 (2000).
Kim, Y. J. et al. Transcriptional activation of Cidec by PPARgamma2 in adipocyte. Biochem Biophys Res Commun 377, 297-302 (2008).
Kiss, M. et al. Retinoid X receptor suppresses a metastasis-promoting transcriptional program in myeloid cells via a ligand-insensitive mechanism. Proc Natl Acad Sci USA 114, 10725-10730 (2017).
Knizkova D, Pribikova M, Draberova H, Semberova T, Trivic T, Synackova A, et al. CMTM4 is a subunit of the IL-17 receptor and mediates autoimmune pathology. Nat Immunol 2022; 23:1644-52
Kodumudi, K. N. et al. A novel chemoimmunomodulating property of docetaxel: suppression of myeloid-derived suppressor cells in tumor bearers. Clin Cancer Res 16, 4583-4594 (2010).
Li T, Cheng Y, Wang P, Wang W. Hu F, Mo X, et al. CMTM4 is frequently downregulated and functions as a tumour suppressor in clear cell renal cell carcinoma. J Exp Clin Cancer Res 2015; 34:122
Liao B C, Lin C C, Yang J C. Second and third-generation epidermal growth factor receptor tyrosine kinase inhibitors in advanced nonsmall cell lung cancer. Curr Opin Oncol 2015; 27:94-101
Lio C J & Huang S C Circles of Life: linking metabolic and epigenetic cycles to immunity. Immunology 161, 165-174 (2020).
Luo, Y. & Knudson, M. J. Mycobacterium bovis bacillus Calmette-Guerin-induced macrophage cytotoxicity against bladder cancer cells. Clin Dev Immunol 2010, 357591 (2010).
Ma G, Pan P Y, Eisenstein S, Divino C M, Lowell C A, Takai T, et al. Paired immunoglobin-like receptor-B regulates the suppressive function and fate of myeloid-derived suppressor cells. Immunity 2011; 34:385-95
Ma, G. et al. Paired immunoglobin-like receptor-B regulates the suppressive function and fate of myeloid-derived suppressor cells. Immunity 34, 385-395 (2011).
Maiorino L, Egeblad M. Tumours pick the path to cancer inflammation. Nat Cell Biol 2019; 21:1055-7
Manning B D, Toker A. AKT/PKB Signaling: Navigating the Network. Cell 2017; 169:381-405
Mantovani, A., Marchesi, F., Malesci, A., Laghi, L. & Allavena, P. Tumour-associated macrophages as treatment targets in oncology. Nat Rev Clin Oncol 14, 399-416 (2017).
McNelis J C & Olefsky J M Macrophages, Immunity, and Metabolic Disease. Immunity 41, 36-48 (2014).
Mehan M R, Williams S A, Siegfried J M, Bigbee W L, Weissfeld J L, Wilson D O, et al. Validation of a blood protein signature for non-small cell lung cancer. Clin Proteomics 2014; 11:32
Mezzadra R, Sun C, Jae L T, Gomez-Eerland R, de Vries E, Wu W, et al. Identification of CMTM6 and CMTM4 as PD-L1 protein regulators. Nature 2017; 549:106-10
Mineo C. James G L, Smart E J, Anderson R G. Localization of epidermal growth factor-stimulated Ras/Raf-1 interaction to caveolae membrane. J Biol Chem 1996; 271:11930-5
Moser, A. R., Pitot, H. C. & Dove, W. F. A dominant mutation that predisposes to multiple intestinal neoplasia in the mouse. Science 247, 322-324 (1990).
Murray, P. J. et al. Macrophage activation and polarization: nomenclature and experimental guidelines. Immunity 41, 14-20 (2014).
Nicolas-Avila, J. A., Adrover, J. M. & Hidalgo, A. Neutrophils in Homeostasis, Immunity, and Cancer. Immunity 46, 15-28 (2017).
Ochoa A C, Zea A H, Hemandez C, Rodriguez P C. Arginase, prostaglandins, and myeloid-derived suppressor cells in renal cell carcinoma. Clin Cancer Res 2007; 13:721s-6s
Oyadomari S, Harding H P, Zhang Y, Oyadomari M, Ron D. Dephosphorylation of translation initiation factor 2a enhances glucose tolerance and attenuates hepatosteatosis in mice. Cell Metabolism. 2008; 7(6):520-532.
Ozao-Choy J, Ma G, Kao J, Wang G X, Meseck M, Sung M, et al. The novel role of tyrosine kinase inhibitor in the reversal of immune suppression and modulation of tumor microenvironment for immune-based cancer therapies. Cancer Res 2009; 69:2514-22
Pan P Y, Ma G, Weber K J, Ozao-Choy J, Wang G, Yin B, et al. Immune stimulatory receptor CD40 is required for T-cell suppression and T regulatory cell activation mediated by myeloid-derived suppressor cells in cancer. Cancer Res 2010; 70:99-108
Pan P Y, Ozao J, Zhou Z, Chen S H. Advancements in immune tolerance. Adv Drug Deliv Rev 2008; 60:91-105
Pan P Y, Wang G X, Yin B, Ozao J, Ku T, Divino C M, et al. Reversion of immune tolerance in advanced malignancy: modulation of myeloid-derived suppressor cell development by blockade of stem-cell factor function. Blood 2008; 111:219-28
Petryszak, R. et al. Expression Atlas update—an integrated database of gene and protein expression in humans, animals and plants. Nucleic Acids Res 44, D746-752 (2016).
Piccolo, V. et al. Opposing macrophage polarization programs show extensive epigenomic and transcriptional cross-talk. Nat Immunol 18, 530-540 (2017).
Pilla-Moffett, D., Barber, M. F., Taylor, G. A. & Coers, J. Interferon-Inducible GTPases in Host Resistance, Inflammation and Disease. J Mol Biol 428, 3495-3513 (2016).
Plate M, Li T, Wang Y, Mo X, Zhang Y, Ma D, et al. Identification and characterization of CMTM4, a novel gene with inhibitory effects on HeLa cell growth through Inducing G2/M phase accumulation. Mol Cells 2010; 29:355-61
Ranjit, S. et al. Regulation of fat specific protein 27 by isoproterenol and TNF-alpha to control lipolysis in murine adipocytes. J Lipid Res 52, 221-236 (2011).
Rhee, I. Diverse macrophages polarization in tumor microenvironment. Arch Pharm Res (2016).
Rosadini, C. V. & Kagan, J. C. Microbial strategies for antagonizing Toll-like-receptor signal transduction. Curr Opin Immunol 32, 61-70 (2015).
Sangaletti, S. et al. Mesenchymal Transition of High-Grade Breast Carcinomas Depends on Extracellular Matrix Control of Myeloid Suppressor Cell Activity. Cell Rep 17, 233-248 (2016).
Sangaletti, S. et al. SPARC Is a New Myeloid-Derived Suppressor Cell Marker Licensing Suppressive Activities. Front Immunol 10, 1369 (2019).
Schwitalla, S. et al. Loss of p53 in enterocytes generates an inflammatory microenvironment enabling invasion and lymph node metastasis of carcinogen-induced colorectal tumors. Cancer Cell 23, 93-106 (2013).
Semerad C L, Liu F, Gregory A D, Stumpf K, Link D C. G-CSF is an essential regulator of neutrophil trafficking from the bone marrow to the blood. Immunity 2002; 17:413-23
Shen X, Zhang K, Kaufman R J. The unfolded protein response—a stress signaling pathway of the endoplasmic reticulum. Journal of Chemical Neuroanatomy. 2004; 28(1-2):79-92.
Sun, L. et al. Expression of transforming growth factor beta type II receptor leads to reduced malignancy in human breast cancer MCF-7 cells. J Biol Chem 269, 26449-26455 (1994).
Tang F, Wang Y, Hemmings B A, Ruegg C, Xue G. PKB/Akt-dependent regulation of inflammation in cancer. Semin Cancer Biol 2018; 48:62-9
Taniguchi K, Karin M. NF-kappaB, inflammation, immunity and cancer: coming of age. Nat Rev Immunol 2018; 18:309-24
Udalova, I. A., Mantovani, A. & Feldmann, M. Macrophage heterogeneity in the context of rheumatoid arthritis. Nat Rev Rheumatol 12, 472-485 (2016).
van der Touw, W., Chen, H. M., Pan, P. Y. & Chen, S. H. LILRB receptor-mediated regulation of myeloid cell maturation and function. Cancer Immunol Immunother 66, 1079-1087 (2017).
Vats, D. et al. Oxidative metabolism and PGC-1beta attenuate macrophage-mediated inflammation. Cell Metab 4, 13-24 (2006).
Wagsater, D. et al. ADAMTS-4 and -8 are inflammatory regulated enzymes expressed in macrophage-rich areas of human atherosclerotic plaques. Atherosclerosis 196, 514-522 (2008).
Wang Y, Li J, Cui Y, Li T, Ng K M, Geng H, et al. CMTM3, located at the critical tumor suppressor locus 16q22.1, is silenced by CpG methylation in carcinomas and inhibits tumor cell growth through inducing apoptosis. Cancer Res 2009; 69:5194-201
Wang, N., Liang, H. & Zen, K. Molecular mechanisms that influence the macrophage m1-m2 polarization balance. Front Immunol 5, 614 (2014).
Welte T, Kim I S, Tian L, Gao X, Wang H, Li J, et al. Oncogenic mTOR signalling recruits myeloid-derived suppressor cells to promote tumour initiation. Nat Cell Biol 2016; 18:632-44
Wieser, V., Gerner, R., Moschen, A. R. & Tilg, H. Liver complications in inflammatory bowel diseases. Dig Dis 31, 233-238 (2013).
Wynn, T. A., Chawla, A. & Pollard, J. W. Macrophage biology in development, homeostasis and disease. Nature 496, 445-455 (2013).
Yaddanapudi, K. et al. Control of tumor-associated macrophage alternative activation by macrophage migration inhibitory factor. J Immunol 190, 2984-2993 (2013).
Yang L, DeBusk L M, Fukuda K, Fingleton B, Green-Jarvis B, Shyr Y, et al. Expansion of myeloid immune suppressor Gr+CD11b+ cells in tumor-bearing host directly promotes tumor angiogenesis. Cancer Cell 2004; 6:409-21
Yang W C, Ma G, Chen S H, Pan P Y. Polarization and reprogramming of myeloid-derived suppressor cells. J Mol Cell Biol 2013; 5:207-9
Zhang L, Xie B, Qiu Y, Jing D, Zhang J, Duan Y, et al. Rab25-Mediated EGFR Recycling Causes Tumor Acquired Radioresistance. iScience 2020; 23:100997

### F. SEQUENCES

SEQ ID NO: 1
UUAGAUUCCAGUUGAUCUGGG
SEQ ID NO: 2
UGUUAGAUUCCAGUUGAUCUG
SEQ ID NO: 3
ACCAAAUCUGUUAGAUUCCAG
SEQ ID NO: 4
AGAAAACUUGAUUAGAAGGAC
SEQ ID NO: 5
AAGAAAACUUGAUUAGAAGGA
SEQ ID NO: 6
AGAAAGAAAACUUGAUUAGAA
SEQ ID NO: 7
UGAAUUUUUACCAAACAGGAC
SEQ ID NO: 8
AAGUGAAUUUUUACCAAACAG
SEQ ID NO: 9
UUUAUUAAGGUUUUGACUCAU
SEQ ID NO: 10
UAUACUUCCCUUCUCAAUGCC
SEQ ID NO: 11
CTTGATTAGAAGGACGGTT
SEQ ID NO: 12
AGAUCAACUGGAACCUGACAGAUUU
SEQ ID NO: 13
GCCGUGAUAUUUGGCUUCUUGGCAA
SEQ ID NO: 14
GGCCCUGAUUGCGUUCAUCAUCUGCAUA

1. A method of detecting a cancer in a subject comprising obtaining a tissue sample from the subject and measuring the expression level of Chemokine-like factor (CKLF)-like MARVEL transmembrane domain-containing member 4 (CMTM4) relative to a control, wherein an increase in the expression of CMTM4 relative to the control indicates the presence of a cancer.

2. A method of assessing whether a cancer in a subject is metastatic comprising obtaining a cancerous tissue sample from a tumor microenvironment in the subject and measuring the expression level of Chemokine-like factor (CKLF)-like MARVEL transmembrane domain-containing member 4 (CMTM4) in the tissue sample relative to a control, wherein an increase in the expression level of CMTM4 relative to the control indicates the cancer is metastatic.

3. The method of claim 1, wherein the cancer is a cholangiocarcinoma (CHOL), colon adenocarcinoma (COAD), esophageal carcinoma (ESCA), kidney chromophobe (KICH), pheochromocytoma and paraganglioma (PCPG), prostate adenocarcinoma (PRAD), rectum adenocarcinoma (READ), and thymoma (THYM), breast cancer, colon cancer, melanoma, prostate cancer, a glioma, kidney cancer, or lung adenocarcinoma.

4. The method of claim 1, wherein a cancer is detected, or a cancer is found to be metastatic, the method further comprises administering to the subject an agent that inhibits CMTM4.

5. The method of claim 4, wherein the agent comprises a miRNA, shRNA, siRNA, peptide, small molecule, or antibody that binds to CMTM4, inhibits the activity of CMTM4, or inhibits the expression of CMTM4.

6. The method of claim 5, wherein the agent comprises C29H38O4 (Celastrol), Ellipticine, C54H78N2O17 (Lobophorin F), C19H14O7 (5-Methoxysterigmatocysin), C16H12O7 (Isorhamnetin), C21H26N2O3 (Tabernaemontanin), C27H34O10 (Verrucarin A 9,10-epoxide), C10H12N4O5 (Inosine), C16H12O7, C18H23NO6, C25H33N3O·ClH, C16H12O7 (Rhamnetin), C12H13N5O4 (Toyocamycin), C17H19N3O4 (Anthramycin methyl ether), C21H22N2O3 (Paquinimod), C20H18N2O5·Na (Camptothecin sodium), C18H16O8 (Rosmarinic Acid), C10H14O3, C9H16N2O6 (Tetrahydrouridine), C10H14O3 (Mephenesin), C10H14O3 (4-Ethoxy-3-methoxybenzyl alcohol), C10H14O3 (5-tert-butyl-2-methyl-3-furoic acid), C21H20F3NO6, C23H32N2O6, C15H10N2O, C36H48N2O12 (Rhoodomycin A), C16H24O5 (Ocalicine subst.), C14H25N3O9 (Kasugamycin), C4H6O2 (Crotonic Acid), C21H26N2O3 (Vincamine), Fumitremorgin C, C10H12N4O5 (Formycin B), C18H23NO6 (Swazine), C25H33N3O·ClH (Butylcycloheptylprodiginine Hydrochloride), C18H22N2O4·C6H8O7 (Quinocarcin monocitrate), C23H28N2O5, C11H13NO2·BrH, C21H24N2O3 (Strychninic acid), C21H22N2O3 (Pseudostrychnine), C20H18N2O5·Na (Camptothecin sodium), C18H16O8 (Centaureidin), C10H14O3 (Ramulosin), C19H26N2·C4H6O6, C15H10N2O6 (Lomondomycin), C21H26N2O3 (Pseudoyohimbine), or C26H28O5 (Glycyrrhizol A).

7. The method of claim 5, wherein the agent is an siRNA and wherein the siRNA comprises UUAGAUUCCAGUUGAUCUGGG (SEQ ID NO: 1), UGUUAGAUUCCAGUUGAUCUG (SEQ ID NO: 2), ACCAAAUCUGUUAGAUUCCAG (SEQ ID NO: 3), AGAAAACUUGAUUAGAAGGAC (SEQ ID NO: 4), AAGAAAACUUGAUUAGAAGGA (SEQ ID NO: 5), AGAAAGAAAACUUGAUUAGAA (SEQ ID NO: 6), UGAAUUUUUACCAAACAGGAC (SEQ ID NO: 7), AAGUGAAUUUUUACCAAACAG (SEQ ID NO: 8), UUUAUUAAGGUUUUGACUCAU (SEQ ID NO: 9), UAUACUUCCCUUCUCAAUGCC (SEQ ID NO: 10), CTTGATTAGAAGGACGGTT (SEQ ID NO: 11), AGAUCAACUGGAACCUGACAGAUUU (SEQ ID NO: 12), GCCGUGAUAUUUGGCUUCUUGGCAA (SEQ ID NO: 13), or GGCCCUGAUUGCGUUCAUCAUCUGCAUA (SEQ ID NO: 14).

8. A method of treating a cancer and/or metastasis in a subject comprising administering to the subject an agent that inhibits CMTM4.

9. The method of claim 8, wherein the agent comprises a miRNA, shRNA, siRNA, peptide, small molecule, or antibody that binds to CMTM4, inhibits the activity of CMTM4, or inhibits the expression of CMTM4.

10. The method of claim 9, wherein the agent comprises C29H38O4 (Celastrol), Ellipticine, C54H78N2O17 (Lobophorin F), C19H14O7 (5-Methoxysterigmatocysin), C16H12O7 (Isorhamnetin), C21H26N2O3 (Tabernaemontanin), C27H34O10 (Verrucarin A 9,10-epoxide), C10H12N4O5 (Inosine), C16H12O7, C18H23NO6, C25H33N3O·ClH, C16H12O7 (Rhamnetin), C12H13N5O4 (Toyocamycin), C17H19N3O4 (Anthramycin methyl ether), C21H22N2O3 (Paquinimod), C20H18N2O5·Na (Camptothecin sodium), C18H16O8 (Rosmarinic Acid), C10H14O3, C9H16N2O6 (Tetrahydrouridine), C10H14O3 (Mephenesin), C10H14O3 (4-Ethoxy-3-methoxybenzyl alcohol), C10H14O3 (5-tert-butyl-2-methyl-3-furoic acid), C21H20F3NO6, C23H32N2O6, C15H10N2O, C36H48N2O12 (Rhoodomycin A), C16H24O5 (Ocalicine subst.), C14H25N3O9 (Kasugamycin), C4H6O2 (Crotonic Acid), C21H26N2O3 (Vincamine), Fumitremorgin C, C10H12N4O5 (Formycin B), C18H23NO6 (Swazine), C25H33N3O·ClH (Butylcycloheptylprodiginine Hydrochloride), C18H22N2O4·C6H8O7 (Quinocarcin monocitrate), C23H28N2O5, C11H13NO2·BrH, C21H24N2O3 (Strychninic acid), C21H22N2O3 (Pseudostrychnine), C20H18N2O5·Na (Camptothecin sodium), C18H16O8 (Centaureidin), C10H14O3 (Ramulosin), C19H26N2·C4H6O6, C15H10N2O6 (Lomondomycin), C21H26N2O3 (Pseudoyohimbine), or C26H28O5 (Glycyrrhizol A).

11. The method of claim 9, wherein the agent is an siRNA and wherein the siRNA comprises UUAGAUUCCAGUUGAUCUGGG (SEQ ID NO: 1), UGUUAGAUUCCAGUUGAUCUG (SEQ ID NO: 2), ACCAAAUCUGUUAGAUUCCAG (SEQ ID NO: 3), AGAAAACUUGAUUAGAAGGAC (SEQ ID NO: 4), AAGAAAACUUGAUUAGAAGGA (SEQ ID NO: 5), AGAAAGAAAACUUGAUUAGAA (SEQ ID NO: 6), UGAAUUUUUACCAAACAGGAC (SEQ ID NO: 7), AAGUGAAUUUUUACCAAACAG (SEQ ID NO: 8), UUUAUUAAGGUUUUGACUCAU (SEQ ID NO: 9), UAUACUUCCCUUCUCAAUGCC (SEQ ID NO: 10), CTTGATTAGAAGGACGGTT (SEQ ID NO: 11), AGAUCAACUGGAACCUGACAGAUUU (SEQ ID NO: 12), GCCGUGAUAUUUGGCUUCUUGGCAA (SEQ ID NO: 13), or GGCCCUGAUUGCGUUCAUCAUCUGCAUA (SEQ ID NO: 14).

12. The method of treating a cancer of claim 8, wherein the cancer is cholangiocarcinoma (CHOL), colon adenocarcinoma (COAD), esophageal carcinoma (ESCA), kidney chromophobe (KICH), pheochromocytoma and paraganglioma (PCPG), prostate adenocarcinoma (PRAD), rectum adenocarcinoma (READ), and thymoma (THYM), breast cancer, colon cancer, melanoma, prostate cancer, a glioma, kidney cancer, or lung adenocarcinoma.

13. The method of treating a cancer and/or metastasis of claim 8, further comprise the administration of anti-inflammatory agents and/or antibodies that bind to neutrophils.

14. The method of treating a cancer and/or metastasis of claim 8, further comprise the administration of an epidermal growth factor receptor (EGFR) inhibitor or a platelet-derived growth factor receptor A (PDGFRa) inhibitor.

15. The method of treating a cancer and/or metastasis of claim 14, wherein the EGFR inhibitor is selected from the group consisting of erlotinib, osimertinib, neratinib, gefitinib, cetuximab, pantibumumab, dacomitinib, lapatinib, necitumumab, mobocertinib, and vandetanib.

16. The method of treating a cancer and/or metastasis of claim 14, wherein the PDGFRa inhibitor is selected from the group consisting of avapritinib, imatinib, and ripretinib.

17. A method of decreasing immunosuppressive activity in a tumor microenvironment of a cancer in a subject comprising administering to the microenvironment an agent that inhibits CMTM4.

18. The method of claim 17, wherein the agent comprises a miRNA, shRNA, siRNA, peptide, small molecule, or antibody that binds to CMTM4, inhibits the activity of CMTM4, or inhibits the expression of CMTM4.

19. The method of claim 18, wherein the agent comprises C29H38O4 (Celastrol), Ellipticine, C54H78N2O17 (Lobophorin F), C19H14O7 (5-Methoxysterigmatocysin), C16H12O7 (Isorhamnetin), C21H26N2O3 (Tabernaemontanin), C27H34O10 (Verrucarin A 9,10-epoxide), C10H12N4O5 (Inosine), C16H12O7, C18H23NO6, C25H33N3O·ClH, C16H12O7 (Rhamnetin), C12H13N5O4 (Toyocamycin), C17H19N3O4 (Anthramycin methyl ether), C21H22N2O3 (Paquinimod), C20H18N2O5·Na (Camptothecin sodium), C18H16O8 (Rosmarinic Acid), C10H14O3, C9H16N2O6 (Tetrahydrouridine), C10H14O3 (Mephenesin), C10H14O3 (4-Ethoxy-3-methoxybenzyl alcohol), C10H14O3 (5-tert-butyl-2-methyl-3-furoic acid), C21H20F3NO6, C23H32N2O6, C15H10N2O, C36H48N2O12 (Rhoodomycin A), C16H24O5 (Ocalicine subst.), C14H25N3O9 (Kasugamycin), C4H6O2 (Crotonic Acid), C21H26N2O3 (Vincamine), Fumitremorgin C, C10H12N4O5 (Formycin B), C18H23NO6 (Swazine), C25H33N3O·ClH (Butylcycloheptylprodiginine Hydrochloride), C18H22N2O4·C6H8O7 (Quinocarcin monocitrate), C23H28N2O5, C11H13NO2·BrH, C21H24N2O3 (Strychninic acid), C21H22N2O3 (Pseudostrychnine), C20H18N2O5·Na (Camptothecin sodium), C18H16O8 (Centaureidin), C10H14O3 (Ramulosin), C19H26N2·C4H6O6, C15H10N2O6 (Lomondomycin), C21H26N2O3 (Pseudoyohimbine), or C26H28O5 (Glycyrrhizol A).

20. The method of claim 18, wherein the agent is an siRNA and wherein the siRNA comprises UUAGAUUCCAGUUGAUCUGGG (SEQ ID NO: 1), UGUUAGAUUCCAGUUGAUCUG (SEQ ID NO: 2), ACCAAAUCUGUUAGAUUCCAG (SEQ ID NO: 3), AGAAAACUUGAUUAGAAGGAC (SEQ ID NO: 4), AAGAAAACUUGAUUAGAAGGA (SEQ ID NO: 5), AGAAAGAAAACUUGAUUAGAA (SEQ ID NO: 6), UGAAUUUUUACCAAACAGGAC (SEQ ID NO: 7), AAGUGAAUUUUUACCAAACAG (SEQ ID NO: 8), UUUAUUAAGGUUUUGACUCAU (SEQ ID NO: 9), UAUACUUCCCUUCUCAAUGCC (SEQ ID NO: 10), CTTGATTAGAAGGACGGTT (SEQ ID NO: 11), AGAUCAACUGGAACCUGACAGAUUU (SEQ ID NO: 12), GCCGUGAUAUUUGGCUUCUUGGCAA (SEQ ID NO: 13), or GGCCCUGAUUGCGUUCAUCAUCUGCAUA (SEQ ID NO: 14).

21. The method of claim 17, wherein the cancer is cholangiocarcinoma (CHOL), colon adenocarcinoma (COAD), esophageal carcinoma (ESCA), kidney chromophobe (KICH), pheochromocytoma and paraganglioma (PCPG), prostate adenocarcinoma (PRAD), rectum adenocarcinoma (READ), and thymoma (THYM), breast cancer, colon cancer, melanoma, prostate cancer, a glioma, kidney cancer, or lung adenocarcinoma.

22. A method of decreasing expression of IL-6 and/or CMTM4 in a tumor microenvironment in a subject, the method comprising administering to the subject C29H38O4 (Celastrol), Ellipticine, C54H78N2O17 (Lobophorin F), C19H14O7 (5-Methoxysterigmatocysin), C16H12O7 (Isorhamnetin), C21H26N2O3 (Tabernaemontanin), C27H34O10 (Verrucarin A 9,10-epoxide), C10H12N4O5 (Inosine), C16H12O7, C18H23NO6, C25H33N3O·ClH, C16H12O7 (Rhamnetin), C12H13N5O4 (Toyocamycin), C17H19N3O4 (Anthramycin methyl ether), C21H22N2O3 (Paquinimod), C20H18N2O5·Na (Camptothecin sodium), C18H16O8 (Rosmarinic Acid), C10H14O3, C9H16N2O6 (Tetrahydrouridine), C10H14O3 (Mephenesin), C10H14O3 (4-Ethoxy-3-methoxybenzyl alcohol), C10H14O3 (5-tert-butyl-2-methyl-3-furoic acid), C21H20F3NO6, C23H32N2O6, C15H10N2O, C36H48N2O12 (Rhoodomycin A), C16H24O5 (Ocalicine subst.), C14H25N3O9 (Kasugamycin), C4H6O2 (Crotonic Acid), C21H26N2O3 (Vincamine), Fumitremorgin C, C10H12N4O5 (Formycin B), C18H23NO6 (Swazine), C25H33N3O·ClH (Butylcycloheptylprodiginine Hydrochloride), C18H22N2O4·C6H8O7 (Quinocarcin monocitrate), C23H28N2O5, C11H13NO2·BrH, C21H24N2O3 (Strychninic acid), C21H22N2O3 (Pseudostrychnine), C20H18N2O5·Na (Camptothecin sodium), C18H16O8 (Centaureidin), C10H14O3 (Ramulosin), C19H26N2·C4H6O6, C15H10N2O6 (Lomondomycin), C21H26N2O3 (Pseudoyohimbine), or C26H28O5 (Glycyrrhizol A) or a siRNA and wherein the siRNA comprises
SEQ ID NO: 1
UUAGAUUCCAGUUGAUCUGGG,
SEQ ID NO: 2
UGUUAGAUUCCAGUUGAUCUG,
SEQ ID NO: 3
ACCAAAUCUGUUAGAUUCCAG,
SEQ ID NO: 4
AGAAAACUUGAUUAGAAGGAC,
SEQ ID NO: 5
AAGAAAACUUGAUUAGAAGGA,
SEQ ID NO: 6
AGAAAGAAAACUUGAUUAGAA,
SEQ ID NO: 7
UGAAUUUUUACCAAACAGGAC,
SEQ ID NO: 8
AAGUGAAUUUUUACCAAACAG,
SEQ ID NO: 9
UUUAUUAAGGUUUUGACUCAU,
SEQ ID NO: 10
UAUACUUCCCUUCUCAAUGCC,
SEQ ID NO: 11
CTTGATTAGAAGGACGGTT,
SEQ ID NO: 12
AGAUCAACUGGAACCUGACAGAUUU,
SEQ ID NO: 13
GCCGUGAUAUUUGGCUUCUUGGCAA,
or
SEQ ID NO: 14
GGCCCUGAUUGCGUUCAUCAUCUGCAUA.

23. A method of increasing MHCII expression in a tumor microenvironment in a subject, the method comprising administering to the subject an agent that inhibits CMTM4 expression.

24. (canceled)

25. The method of claim 23, wherein the agent comprises a miRNA, shRNA, siRNA, peptide, small molecule, or antibody that binds to CMTM4, inhibits the activity of CMTM4, or inhibits the expression of CMTM4.

26. The method of claim 25, wherein the agent comprises C29H38O4 (Celastrol), Ellipticine, C54H78N2O17 (Lobophorin F), C19H14O7 (5-Methoxysterigmatocysin), C16H12O7 (Isorhamnetin), C21H26N2O3 (Tabernaemontanin), C27H34O10 (Verrucarin A 9,10-epoxide), C10H12N4O5 (Inosine), C16H12O7, C18H23NO6, C25H33N3O·ClH, C16H12O7 (Rhamnetin), C12H13N5O4 (Toyocamycin), C17H19N3O4 (Anthramycin methyl ether), C21H22N2O3 (Paquinimod), C20H18N2O5·Na (Camptothecin sodium), C18H16O8 (Rosmarinic Acid), C10H14O3, C9H16N2O6 (Tetrahydrouridine), C10H14O3 (Mephenesin), C10H14O3 (4-Ethoxy-3-methoxybenzyl alcohol), C10H14O3 (5-tert-butyl-2-methyl-3-furoic acid), C21H20F3NO6, C23H32N2O6, C15H10N2O, C36H48N2O12 (Rhoodomycin A), C16H24O5 (Ocalicine subst.), C14H25N3O9 (Kasugamycin), C4H6O2 (Crotonic Acid), C21H26N2O3 (Vincamine), Fumitremorgin C, C10H12N4O5 (Formycin B), C18H23NO6 (Swazine), C25H33N3O·ClH (Butylcycloheptylprodiginine Hydrochloride), C18H22N2O4·C6H8O7 (Quinocarcin monocitrate), C23H28N2O5, C11H13NO2·BrH, C21H24N2O3 (Strychninic acid), C21H22N2O3 (Pseudostrychnine), C20H18N2O5·Na (Camptothecin sodium), C18H16O8 (Centaureidin), C10H14O3 (Ramulosin), C19H26N2·C4H6O6, C15H10N2O6 (Lomondomycin), C21H26N2O3 (Pseudoyohimbine), or C26H28O5 (Glycyrrhizol A).

27. The method of claim 25, wherein the agent is an siRNA and wherein the siRNA comprises UUAGAUUCCAGUUGAUCUGGG (SEQ ID NO: 1), UGUUAGAUUCCAGUUGAUCUG (SEQ ID NO: 2), ACCAAAUCUGUUAGAUUCCAG (SEQ ID NO: 3), AGAAAACUUGAUUAGAAGGAC (SEQ ID NO: 4), AAGAAAACUUGAUUAGAAGGA (SEQ ID NO: 5), AGAAAGAAAACUUGAUUAGAA (SEQ ID NO: 6), UGAAUUUUUACCAAACAGGAC (SEQ ID NO: 7), AAGUGAAUUUUUACCAAACAG (SEQ ID NO: 8), UUUAUUAAGGUUUUGACUCAU (SEQ ID NO: 9), UAUACUUCCCUUCUCAAUGCC (SEQ ID NO: 10), CTTGATTAGAAGGACGGTT (SEQ ID NO: 11), AGAUCAACUGGAACCUGACAGAUUU (SEQ ID NO: 12), GCCGUGAUAUUUGGCUUCUUGGCAA (SEQ ID NO: 13), or GGCCCUGAUUGCGUUCAUCAUCUGCAUA (SEQ ID NO: 14).


SEQUENCE LISTING (14 sequences)

SEQ ID NO: 1 - RNA, 21 nt, synthetic construct
  source 1..21: mol_type = other RNA
ttagattccagttgatctggg

SEQ ID NO: 2 - RNA, 21 nt, synthetic construct
  source 1..21: mol_type = other RNA
tgttagattccagttgatctg

SEQ ID NO: 3 - RNA, 21 nt, synthetic construct
  source 1..21: mol_type = other RNA
accaaatctgttagattccag

SEQ ID NO: 4 - RNA, 21 nt, synthetic construct
  source 1..21: mol_type = other RNA
agaaaacttgattagaaggac

SEQ ID NO: 5 - RNA, 21 nt, synthetic construct
  source 1..21: mol_type = other RNA
aagaaaacttgattagaagga

SEQ ID NO: 6 - RNA, 21 nt, synthetic construct
  source 1..21: mol_type = other RNA
agaaagaaaacttgattagaa

SEQ ID NO: 7 - RNA, 21 nt, synthetic construct
  source 1..21: mol_type = other RNA
tgaatttttaccaaacaggac

SEQ ID NO: 8 - RNA, 21 nt, synthetic construct
  source 1..21: mol_type = other RNA
aagtgaatttttaccaaacag

SEQ ID NO: 9 - RNA, 21 nt, synthetic construct
  source 1..21: mol_type = other RNA
tttattaaggttttgactcat

SEQ ID NO: 10 - RNA, 21 nt, synthetic construct
  source 1..21: mol_type = other RNA
tatacttcccttctcaatgcc

SEQ ID NO: 11 - RNA, 19 nt, synthetic construct
  source 1..19: mol_type = other RNA
cttgattagaaggacggtt

SEQ ID NO: 12 - RNA, 25 nt, synthetic construct
  source 1..25: mol_type = other RNA
agatcaactggaacctgacagattt

SEQ ID NO: 13 - RNA, 25 nt, synthetic construct
  source 1..25: mol_type = other RNA
gccgtgatatttggcttcttggcaa

SEQ ID NO: 14 - RNA, 28 nt, synthetic construct
  source 1..28: mol_type = other RNA
ggccctgattgcgttcatcatctgcata